(19) United States
(12) Patent Application Publication
(10) Pub. No.: US 2024/0159780 A1
(43) Pub. Date: May 16, 2024

(54) PREDICTION OF AMOUNT OF IN VIVO DOPAMINE ETC., AND APPLICATION THEREOF

(71) Applicant: Sumitomo Pharma Co., Ltd., Osaka-shi (JP)

(72) Inventors: Shin TEJIMA, Osaka-shi (JP); Mitsumasa KURITA, Osaka-shi (JP)

(73) Assignee: Sumitomo Pharma Co., Ltd., Osaka-shi (JP)

(21) Appl. No.: 18/549,967

(22) PCT Filed: Mar. 11, 2022
(86) PCT No.: PCT/JP2022/011100

§ 371 Date: Sep. 11, 2023

(30) Foreign Application Priority Data
Mar. 12, 2021 (JP) 2021-040515

(51) Int. Cl. G01N 33/94; A61B 3/10; G16H 50/50

(52) U.S. Cl. CPC G01N 33/9413; A61B 3/10; G16H 50/50; G01N 2800/2835

(57) ABSTRACT

The present disclosure provides methods of evaluating therapeutic or prophylactic agents or other medical technologies for patients with Parkinson's disease being treated with L-DOPA or L-DOPA-related compounds or dopamine agonists. Specifically, it provides a method of evaluating a therapeutic or prophylactic agent or other medical technology for a patient with Parkinson's disease who is being treated with L-DOPA or an L-DOPA-related compound or a dopamine agonist, including A) a step of obtaining ocular information of the patient, and B) a step of calculating an estimated effective amount or effective level of the therapeutic or prophylactic agent or other medical technology from the ocular information.

### TECHNICAL FIELD

The present disclosure relates to methods and the like for predicting the amount of in vivo dopamine or a substance biologically equivalent to dopamine based on ocular information. The present disclosure also relates to methods of estimating the condition of Parkinson's disease patients undergoing treatment with L-DOPA or an L-DOPA-related compound or a dopamine agonist, and the like.

### BACKGROUND ART

Parkinson's disease is one of the progressive neurodegenerative diseases with abnormal extrapyramidal function as the main symptom. Pathologically, dopamine neuronal loss and deposition of Alpha-synuclein in the substantia nigra pars compacta are observed. Clinically, it presents with various motor symptoms such as akinesia, tremor at rest, rigidity, loss of postural reflexes, and the like.

The basis of Parkinson's disease treatment is drug therapy aimed at dopamine replacement in the brain, and drugs containing levodopa (L-DOPA, levodopa), which is a dopamine precursor, are used as the first-choice drug for initial treatment of Parkinson's disease. However, as the disease progresses, motor complications such as motor fluctuations of Parkinson's symptoms, levodopa-induced dyskinesia in Parkinson's disease (hereinafter referred to as “PD-LID” (Parkinson's Disease Levodopa induced dyskinesia) appear in nearly all patients on levodopa treatment.

As typical symptoms of diurnal variations, wearing-off, on-off phenomenon, no-on phenomenon, delayed-on phenomenon, and the like are known. Among them, wearing off is a symptom in which, as mentioned above, when the ability to retain dopamine in the synaptic cleft decreases due to the progression of the disease, the brain dopamine concentration fluctuates according to the blood concentration of levodopa, and as a result, the blood concentration of levodopa is below the safe therapeutic range, and the duration of the effect of levodopa is shortened.

The incidence of PD-LID is 30-50% 5 years after the start of levodopa treatment, increases with progression of the disease, and reaches 50-100% 10 years after the start of treatment. As a typical symptom of PD-LID, peak-dose dyskinesia is known, and it is an involuntary motion which occurs in the face, tongue, neck, extremities, trunk, etc., when the blood concentration of levodopa is high.

The condition of Parkinson's disease patients being treated with L-DOPA is often not known until dyskinesias actually occur. On the other hand, it is known that the amount of L-DOPA in the brain correlates with the onset of dyskinesia, but it is not realistic to measure the amount of L-DOPA in the brain in real time, thus, no realistically feasible method for estimating Parkinson's disease condition in real time has been provided.

### SUMMARY OF THE INVENTION

### Means for Solving the Problem

The present disclosure has been completed by finding that eye movement information serves as an indicator for estimating the amount of in vivo dopamine or a substance biologically equivalent to dopamine and the condition of Parkinson's disease patients.

Accordingly, the present disclosure provides the following.

### (Item 1)

A method for estimating or predicting the presence or absence, amount or level of in vivo dopamine or a substance biologically equivalent to dopamine in a subject, or a variation thereof, based on ocular information in the subject.

### (Item 2)

The method according to item 1, wherein the in vivo dopamine or the substance biologically equivalent to dopamine includes dopamine or a substance biologically equivalent to dopamine in the brain.

### (Item 3)

The method according to item 1 or 2, wherein the ocular information includes at least parameters relating to blinking.

### (Item 4)

A method of evaluating a therapeutic or prophylactic agent or other medical technology for a patient with Parkinson's disease who is being treated with L-DOPA or an L-DOPA-related compound or a dopamine agonist, comprising:
A) a step of obtaining ocular information of the patient; and
B) a step of calculating an estimated effective amount or effective level of the therapeutic or prophylactic agent or other medical technology from the ocular information.

### (Item 5)

The method according to item 4, wherein the step B) comprises:
inputting the ocular information into a trained model, wherein the trained model has trained a correlation between the ocular information and the estimated effective amount or effective level of the therapeutic or prophylactic agent or other medical technology; and
obtaining an output from the trained model.

### (Item 6)

The method according to item 4, wherein the step B) comprises:
a) a step of calculating the estimated presence or absence, amount or level of in vivo dopamine or a substance biologically equivalent to dopamine of the patient based on the ocular information; and
b) a step of calculating an estimated effective amount or effective level of the therapeutic or prophylactic agent or other medical technology from the presence or absence, amount or level, or a variation thereof.

### (Item 7)

The method according to item 6, wherein the step a) comprises:
inputting the ocular information into a trained model, wherein the trained model has trained a correlation between the ocular information and the presence or absence, amount or level of the in vivo dopamine or the substance biologically equivalent to dopamine; and
obtaining an output from the trained model.

### (Item 7A)

The method according to item 7, wherein
the ocular information includes at least one of blink duration, blink frequency classified based on blink duration, long blink frequency and short blink frequency, ratio of long blink frequency to short blink frequency; and
the in vivo dopamine or the substance biologically equivalent to dopamine is L-DOPA in peripheral blood.

### (Item 7B)

The method according to item 7, wherein
the ocular information includes the ratio of short to long blinks, the ratio of blinks whose blink duration is shorter than the first quartile, the median value of the blink duration, the ratio of blinks whose blink duration is between the first and third quartiles, the number of blinks whose blink duration is between the first and third quartiles, the maximum value of the blink duration, the standard deviation of the blink duration, the number of blinks whose blink duration is shorter than the first quartile, the square root value of the blink number, the mean value of the blink duration, the logarithm of the mean value of the blink duration, and the square of the mean value of the blink duration, and
the in vivo dopamine or the substance biologically equivalent to dopamine is L-DOPA in peripheral blood.

### (Item 8)

The method according to any one of items 4 to 7, wherein the therapeutic or prophylactic agent or other medical technology includes L-DOPA or an L-DOPA-related compound, a dopamine agonist, a L-DOPA adjunct, a dopamine neuronal function restoring agent, a dopamine producing cell therapy, a dopamine producing gene therapy, or a surgical therapy.

### (Item 9)

The method according to item 8, wherein the surgical therapy includes deep brain stimulation or stereotactic ablation.

### (Item 10)

The method according to any one of items 4 to 9, further comprising
C) a step of estimating the effect of a medicine having an improving effect on L-DOPA or an L-DOPA-related compound or a dopamine agonist in Parkinson's disease patients being treated with L-DOPA or an L-DOPA-related compound or a dopamine agonist based on the estimated effective amount or effective level.

### (Item 11)

The method according to item 10, wherein the effect of the medicine includes suppressing flactuation and temporal decreases in dopamine amounts in synaptic clefts in the striatum or dopamine agonists when L-DOPA, L-DOPA-related compounds, or dopamine agonists are administered.

### (Item 12)

A method of estimating or predicting the condition of a Parkinson's disease patient being treated with L-DOPA or an L-DOPA-related compound or a dopamine agonist, comprising
A) a step of obtaining ocular information of the patient; and
B) a step of estimating or predicting the patient's condition based on the ocular information.

### (Item 13)

The method according to item 12, wherein the step B) comprises:
inputting the ocular information into a trained model, wherein the trained model has trained a correlation between the ocular information and the patient condition; and
obtaining an output from the trained model.

### (Item 13A)

The method according to item 13, wherein
the ocular information includes at least one of the blink duration, the blink frequency classified based on blink duration, the long blink frequency and short blink frequency, and the ratio of long blink frequency to short blink frequency, and
the patient's condition is a dyskinesia score.

### (Item 13B)

The method according to item 13, wherein
the ocular information includes the ratio of short to long blinks, the median value of the blink duration, the square of the mean value of the blink duration, the ratio of blinks whose blink duration is between the first and third quartiles, the standard deviation of the blink duration, the square root value of the number of blinks, the logarithm of the mean value of the blink duration, the square of the number of blinks, the mean value of the blink duration, and the number of blinks, and
the patient's condition is a dyskinesia score.

### (Item 13C)

The method according to item 13, wherein
the ocular information includes at least one of the blink duration, the blink frequency classified based on blink duration, the long blink frequency and short blink frequency, and the ratio of long blink frequency to short blink frequency, and
the patient is in a condition wherein dyskinesia occurs in 4 or more of the 6 sites of the face, right arm, left arm, trunk, right leg, and left leg.

### (Item 13D)

The method according to item 13, wherein
the ocular information includes the ratio of blinks whose blink duration is shorter than its first quartile, the ratio of short and long blinks, the number of blinks whose blink duration is between the first and third quartiles, the ratio of blinks whose blink duration is between the first and third quartiles, the number of blinks whose blink duration is shorter than its first quartile, the square root of the number of blinks, the median value of the blink duration, and the logarithm of the mean value of the blink duration, and
the patient is in a condition wherein dyskinesia occurs in 4 or more of the 6 sites of the face, right arm, left arm, trunk, right leg, and left leg.

### (Item 14)

The method according to item 12, wherein the step B) comprises:
a) a step of calculating the estimated presence or absence, amount or level of in vivo dopamine or a substance biologically equivalent to dopamine of the patient based on the ocular information; and
b) a step of estimating or predicting the patient's condition from the presence or absence, amount or level, or a variation thereof.

### (Item 15)

The method according to item 14, wherein the step a) comprises:
inputting the ocular information into a trained model, wherein the trained model has trained a correlation between the ocular information and the presence or absence, amount or level of the in vivo dopamine or a substance biologically equivalent to dopamine; and
obtaining an output from the trained model.

### (Item 16)

The method according to any one of items 12 to 15, wherein the condition includes the presence or absence or degree or score of at least one selected from the group consisting of dyskinesia, wearing off, ON-OFF, no-on phenomenon, delayed-on phenomenon, akinesia, resting tremor, muscle rigidity, postural instability, forward leaning posture, freezing phenomenon, sleep disorders, mental/cognitive/behavioral disorders, autonomic disorders and sensory disorders.

### (Item 17)

The method according to item 16, wherein the step B) comprises:
estimating or predicting the presence or absence or degree or score of symptoms of Parkinson's disease or the presence or absence or degree or score of dyskinesia in the patient based on the ocular information.
   (Item 18) The method according to item 15, wherein step b) comprises:
estimating or predicting the patient's condition by comparing an output from the model to one or more thresholds set for the patient.

### (Item 19)

The method according to item 18, further comprising a step of calculating one or more thresholds set for the patient.

### (Item 20)

The method according to item 19, wherein the step of calculating one or more thresholds set for the patient comprises:
obtaining ocular information in the baseline condition of the patient prior to the step A); and
calculating the threshold from the ocular information in the baseline condition.

### (Item 21)

The method according to any one of items 4 to 20, wherein the step A) comprises:
obtaining at least one ocular information source; and
extracting the ocular information from the ocular information source.

### (Item 22)

The method according to item 21, wherein the at least one ocular information source includes optical, physical or electrical information on the muscle or surrounding skin involved in eye or eye movement of the patient, or a combination thereof.

### (Item 23)

The method according to item 22, wherein obtaining the at least one ocular information source includes using at least one selected from the group consisting of an image analysis method, a method using reflected light, a distance measurement method, an electrooculography method, a search coil method, and a probe method, to obtain the at least one ocular information source.

### (Item 24)

The method according to item 22 or 23, wherein extracting the ocular information from the ocular information source includes deriving from the ocular information source a first parameter relating to the eye of the patient and deriving a second parameter relating to the eye of the patient by treating the first parameter, and wherein the ocular information includes the first parameter and/or the second parameter.

### (Item 25)

The method according to item 24, wherein the first parameter includes at least one selected from the group consisting of blink frequency or number of blinks, blink duration, time between blinks, eye closing time, eye opening speed, eye closing speed, eyelid movement width, eyelid opening, and movement distance, movement direction, speed, acceleration, angular velocity, saccade, gliding eye movement, vestibulo-ocular reflex, convergence/divergence, and fixational tremor (ocular tremor, drift, micro saccade) for spontaneous blink, voluntary blink, or reflex blink.

### (Item 26)

The method according to item 24 or item 25, wherein the second parameter includes at least one selected from the group consisting of a function output with the first parameter as an input variable, an intra-division calculated value that is a calculated value in each division when the data of the first parameter is divided into a plurality of divisions by a predetermined threshold, and an inter-division calculated value that is a calculated value of the intra-division calculated value of the plurality of divisions.

### (Item 27)

The method according to item 26, wherein the intra-division calculated value includes various statistics such as maximum value, minimum value, mean value, median value, dispersion, 25% percentile value, 75% percentile value, frequency analysis spectrum, and the like.

### (Item 28)

The method according to item 26 or item 27, wherein the inter-division calculated value includes weighted sums, differences, time differentiations, time integrals, ratios, correlation coefficients, and covariances.

### (Item 29)

The method according to item 24, wherein the first parameter includes blink duration, and the second parameter includes blink frequency classified based on blink duration, or long blink frequency and short blink frequency, or the ratio of long blink frequency to short blink frequency.

### (Item 30)

The method according to item 29, further comprising obtaining criteria for dividing the blink duration from the ocular information source.

### (Item 31)

The method according to item 30, wherein obtaining criteria for dividing the blink duration from the ocular information source includes deriving time-series data of blink frequency and blink duration of each blink from an ocular information source obtained from the patient or another patient, deriving the time-series data of blink frequency into a plurality of data according to the length of the blink duration, calculating a degree of similarity between each of the plurality of classified data and other data among the plurality of classified data adjacent to each other in the blink duration, and setting a threshold for dividing the blink duration based on the similarity.

### (Item 32)

A system for evaluating therapeutic or prophylactic agent or other medical technology for Parkinson's disease patients being treated with L-DOPA or an L-DOPA-related compound or a dopamine agonist, comprising
a means for acquiring ocular information of the patient, and
a means for calculating an estimated effective amount or effective level of the therapeutic or prophylactic agent or other medical technology from the ocular information.

### (Item 33)

A system for estimating or predicting the condition of a Parkinson's disease patient being treated with L-DOPA or an L-DOPA-related compound or a dopamine agonist, comprising
a means for obtaining ocular information of the patient; and
a means for estimating or predicting the patient's condition based on the ocular information.

### (Item 34)

The system according to item 32 or 33, wherein the system is a user device.

### (Item 35)

The system according to item 34, wherein the user device is one information processing device selected from the group consisting of smart phones, tablet computers, smart glasses, smart watches, laptop computers, and desktop computers.

### (Item 36)

The system according to item 34 or item 35, wherein the user device comprises a portion that implements the function of measuring optical information and/or electro-oculography of the muscle or peripheral skin involved in eye or eye movement.

### (Item 37)

The system according to item 32 or item 33, wherein the system includes a user device and a server device, the user device includes an information obtaining means, a transmission means for transmitting the ocular information to the server device, and a receiving means for receiving the result estimated by the estimation means from the server device, and the server device includes a receiving means for receiving the ocular information from the user device, the calculation means, the estimating means, and a transmitting means for transmitting the result estimated by the estimating means to the user device.

### (Item 38)

The system according to item 32 or item 33, wherein the system includes a user device and a server device, the user device includes the information obtaining means, the calculation means, a transmission means for transmitting the calculated presence/absence, amount or level to the server device, and a receiving means for receiving the result estimated by the estimation means from the server device, and the server device includes a receiving means for receiving the calculated presence/absence, quantity or level from the user device the estimating means, and a transmitting means for transmitting the result estimated by the estimating means to the user device.

### (Item 38A)

The system according to item 32 or item 33, including the features according to any one or more of the above items.

### (Item 39)

A program for evaluating therapeutic or prophylactic agent or other medical technology for Parkinson's disease patients being treated with L-DOPA or an L-DOPA-related compound or a dopamine agonist, wherein the program is run on a computer system having a processor, and the program causes the processor to perform processing including
A) a step of obtaining ocular information of the patient; and
B) a step of calculating from the ocular information an estimated effective amount or effective level of the therapeutic or prophylactic agent or other medical technology.

### (Item 40)

A program for estimating or predicting the condition of Parkinson's disease patients being treated with L-DOPA or an L-DOPA-related compound or a dopamine agonist, wherein the program is run on a computer system having a processor, and the program causes the processor to perform processing including
A) a step of obtaining ocular information of the patient; and
B) a step of estimating or predicting the condition of the patient based on the ocular information.

### (Item 41)

The program according to item 39 or item 40, wherein the computer system is a user device.

### (Item 42)

The program according to item 39 or item 40, wherein the computer system is a server device.

### (Item 42A)

A program according to item 39 or item 40, including the features according to any one or more of the above items.

### (Item 42B)

A storage medium storing the program according to any one of items 39 to 42A.

### (Item 43)

A method for managing the health of a Parkinson's disease patient, comprising: performing the method according to item 1, 4 or 12; and applying a treatment to the Parkinson's disease patient if the method determines that the Parkinson's disease patient should be given an additional treatment.

### (Item 43A)

The method according to item 43, including the features according to any one or more of the above items.

### (Item 44)

A method for managing the health of a patient with Parkinson's disease, comprising:
performing the method according to item 1, 4 or 12; and
applying L-DOPA or an L-DOPA-related compound or a dopamine agonist, an L-DOPA adjunct, a dopamine neuronal function restoring agent, a dopamine producing cell therapy, or a dopamine producing gene therapy, or a surgical therapy to the Parkinson's disease patient when it is determined that the L-DOPA or an L-DOPA-related compound or a dopamine agonist, an L-DOPA adjunct, a dopamine neuronal function restoring agent, a dopamine producing cell therapy, or a dopamine producing gene therapy, or a surgical therapy should be applied to the Parkinson's disease patient, by the method.

### (Item 45)

The method according to item 44, wherein the surgical therapy comprises deep brain stimulation or stereotactic ablation.

### (Item 45A)

The method according to item 44, including the features according to any one or more of the above items.

### (Item 46)

A method for managing the health of a Parkinson's disease patient, comprising: performing the method according to item 1, 4 or 12; and issuing an alert regarding a treatment if the method determines that the Parkinson's disease patient should be given an additional treatment.

### (Item 46A)

The method according to item 46, including the features according to any one or more of the above items.

### (Item 47)

A system for health management of a patient with Parkinson's disease, comprising
an estimation or prediction system configured to be able to execute the method according to item 1, 4 or 12, and
a means for applying a treatment to the Parkinson's disease patient if the estimation or prediction system determines that the Parkinson's disease patient should be given an additional treatment.

### (Item 47A)

The system according to item 47, including the features according to any one or more of the above items.

### (Item 48)

A system for health management of a Parkinson's disease patient, comprising
an estimation or prediction system configured to be able to perform the method according to item 1, 4 or 12; and
a means for applying L-DOPA or an L-DOPA-related compound or a dopamine agonist, an L-DOPA adjunct, a dopamine neuronal function restoring agent, a dopamine producing cell therapy, or a dopamine producing gene therapy, or a surgical therapy to the Parkinson's disease patient when it is determined that the L-DOPA or an L-DOPA-related compound or a dopamine agonist, an L-DOPA adjunct, a dopamine neuronal function restoring agent, a dopamine producing cell therapy, or a dopamine producing gene therapy, or a surgical therapy should be applied to the Parkinson's disease patient, by the estimation or prediction system.

### (Item 49)

The system according to item 48, wherein the surgical therapy comprises deep brain stimulation or stereotactic ablation.

### (Item 49A)

The system of item 48, including the features according to any one or more of the above items.

### (Item 50)

A system for health management of a patient with Parkinson's disease, comprising
an estimation or prediction system configured to be able to perform the method according to item 1, 4 or 12, and
a means for issuing an alert relating to a treatment if the estimation or prediction system determines that an additional treatment should be applied to the Parkinson's disease patient.

### (Item 50A)

The system of item 50, including the features according to any one or more of the above items.

### (Item 51)

A program for the health care of patients with Parkinson's disease, wherein the program is run on a computer system having a processor, and the program causes the processor to perform a processing including
performing the method according to item 1, 4 or 12; and
issuing an instruction to perform a treatment on the Parkinson's disease patient if the method determines that the Parkinson's disease patient should be given an additional treatment.

### (Item 51A)

Program according to item 51, including the features according to any one or more of the above items.

### (Item 52)

A program for health care of a patient with Parkinson's disease, wherein the program is run on a computer system comprising a processor, and the program causes the processor to perform a processing including
performing the method according to item 1, 4 or 12; and
issuing an instruction to perform L-DOPA or an L-DOPA-related compound or a dopamine agonist, L-DOPA adjuncts, dopamine neuronal function restoring agents, dopamine producing cell therapy or dopamine producing gene therapy, or if it is determined that a surgical therapy should be applied, to perform L-DOPA or an L-DOPA-related compound or a dopamine agonist, L-DOPA adjuncts, dopamine neuronal function restoring agents, dopamine producing cell therapy or dopamine producing gene therapy, or surgical therapy on the Parkinson's disease patient.

### (Item 53)

The program according to item 52, wherein the surgical therapy comprises deep brain stimulation or stereotactic ablation.

### (Item 53A)

The program according to item 52, including the features according to any one or more of the above items.

### (Item 54)

A program for the health care of patients with Parkinson's disease, wherein the program is run on a computer system having a processor, and the program causes the processor to perform a processing including
performing the method according to item 1, 4 or 12;
and issuing an alert relating to a treatment if the method determines that an additional treatment should be applied to the Parkinson's disease patient.

### (Item 54A)

The program according to item 54, including the features according to any one or more of the above items.

### (Item 54B)

A storage medium storing the program according to any one of items 51 to 54A.

It is contemplated in the present disclosure that one or more of the features described above may be provided in further combinations in addition to the explicit combinations. Still further embodiments and advantages of the present disclosure will be appreciated by those skilled in the art upon reading and understanding the following detailed description, if necessary.

### Effect of the Invention

The present disclosure makes it easy to estimate the presence or absence, amount or level of dopamine or a substance biologically equivalent to dopamine in vivo, such as the presence or absence, amount or level of dopamine or a substance biologically equivalent to dopamine in the brain, and provides a realistically feasible method of estimating the condition of a Parkinson's disease patient in real time. Accordingly, the present disclosure provides techniques that contribute to improving the quality of life of Parkinson's disease patients.

### BRIEF DESCRIPTION OF THE DRAWING

FIG. 1 shows an example of the configuration of a system that is one embodiment of the present disclosure.

FIG. 2 shows an example of the structure of a neural network 20 for constructing a trained model that can be used by the calculation means 12.

FIG. 3 shows an example of the configuration of a system 10′ that is another embodiment of the present disclosure.

FIG. 4 is a diagram showing an example of the configuration of the system 1000 of the present disclosure.

FIG. 5A shows an example of the configuration of the user device 100.

FIG. 5B shows an example of the configuration of the server device 200.

FIG. 6 is a data flow diagram showing exchange of data between the user device 100 and the server device 200 in one embodiment.

FIG. 7 is a data flow diagram showing exchange of data between the user device 100 and the server device 200 in another embodiment.

FIG. 8 is a data flow diagram showing data exchange between the user device 100 and the server device 200 in yet another embodiment.

FIG. 9A shows the result of measuring the amount of change in dopamine over time in the striatum by a microdialysis method after intraperitoneally administering L-dopa to PD-LID model rats and thereafter applying with a tandospirone tape or placebo tape.

FIG. 9B shows the results of calculating the time required for a dopamine release change of 0.2 pg or more. The application of tandospirone tape was found to be effective in prolonging the release time of striatal dopamine compared to the placebo group.

FIG. 9C shows the results of total dopamine release after administration of levodopa. No difference was observed in total dopamine release.

FIG. 10 shows a histogram of the number of blinks in MPTP-induced PD-LID model rhesus monkeys, and (A) shows the number of blinks every 3 minutes when a solvent (0.5% methylcellulose 400 aqueous solution) was orally administered to the model monkeys; (B) shows the number of blinks every 3 minutes when L-DOPA/Benserazide suspended in a solvent (L-DOPA is 22 mg/kg, Benserazide is 1/4 weight of L-DOPA) is orally administered to the model monkeys; and (C) shows the total number of blinks from 30 to 120 minutes after administration when a vehicle was administered and L-DOPA/Benserzide suspension was administered. In (A), no increase in blinking was observed. In (B), eye blinks increased from about 30 minutes after administration of the L-DOPA suspension, peaked at 60 minutes and then declined, but started to increase again from 120 minutes. In (C), the number of blinks tended to increase when L-DOPA was administered.

FIG. 11 shows the number of blinks upon administration of L-DOPA in MPTP-induced PD-LID model rhesus macaques as a 3-minute histogram for each blink duration. Assuming that the blink duration is T (s), (A) T<0.14, (B) 0.14≤T<0.16, (C) 0.16≤T<0.18, (D) 0.18≤T<0.20, (E) 0.20≤T<0.25, and (F) 0.25≤T, respectively. It was shown that the pattern of blink increase by L-DOPA differs for each blink duration.

FIG. 12 shows the relationship between the blink peak and the dyskinesia score peak during L-DOPA administration in MPTP-induced PD-LID model rhesus monkeys, and (A) is a histogram of the number of blinks during L-DOPA administration in model monkeys; (B) shows the dyskinesia score in the same test; and (C) shows the Score of generalized dyskinesia in the same test. In (A), the number of blinks increased from about 30 minutes after the administration of the L-DOPA suspension, peaked at 60 minutes and then attenuated, but started to increase again from 120 minutes. In (B), the dyskinesia score increased about 20 minutes after L-DOPA administration, peaked at 60 minutes after administration, and did not decay until 180 minutes. In (C), the score of generalized dyskinesia increased from 60 minutes after administration, peaked at around 90 minutes, and declined from around 150 minutes. From these, it can be seen that the peak number of blinks occurs before the peak of dyskinesia.

FIG. 13 shows the results of Example 1. In Example 1, a linear regression model was trained to predict the dyskinesia score using the number of blinks for each blink duration in the MPTP-induced PD-LID model rhesus monkey as a feature. The training data and test data were split at 8:2. (A) Changes in dyskinesia score during administration of L-DOPA in model monkeys are shows every 3 minutes. Denote the blink duration as T(s) and the weighted parameter as θ. (B) for T<0.14, θ=0.30, (C) for 0.14≤T<0.16, θ=0.87, (D) for 0.16≤T<0.18, θ=0.20, (E) for 0.18≤T<0.20, θ=−0.066, (F) for 0.20≤T<0.25, θ=−0.33, (G) for 0.25≤T, θ=−0.33. The feature that best predicts dyskinesia was the number of blinks of the blink duration that satisfies 0.14≤T<0.16. (H) Predicted dyskinesia scores were plotted on the vertical axis and true dyskinesia scores on the horizontal axis. The correlation coefficient between the predicted value and the true value in the test data was r=0.97, indicating a very strong correlation.

FIG. 14 shows the results of Example 1. In Example 1, a linear regression model was trained as to predict the Score of generalized dyskinesia (A) in MPTP-induced PD-LID model rhesus monkeys from various features relating to blink duration. The training data and test data were split at 8:2. As a newly adopted feature, certain blink durations T (s) and their ratio were selected, and a parameter weighted by them was θ, then, (B) θ=0.012 for short blinks (T<0.16), (C) θ=−0.16 for long blinks (T≥0.16) and (D) θ=0.26 for the short/long blink ratio. The feature most contributed to the prediction of score of generalized syskinesia was the short/long blink ratio. (H) Predicted generalized dyskinesia scores were plotted on the vertical axis and true Score of generalized dyskinesia on the horizontal axis. Correlation coefficient r=0.77 between the predicted value and the true value in the test data indicates a correlation.

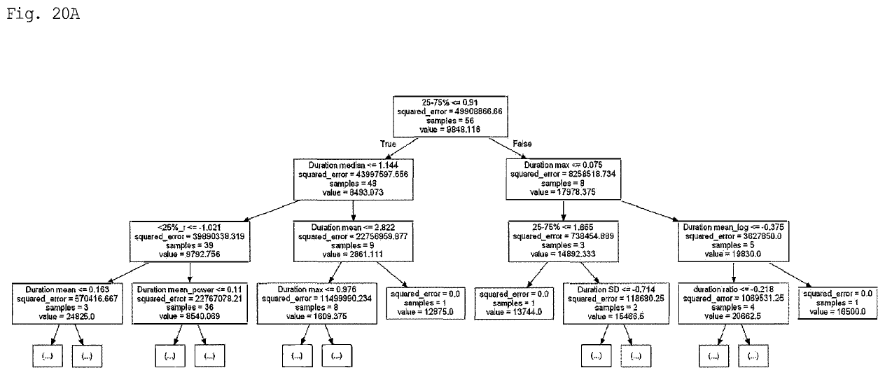

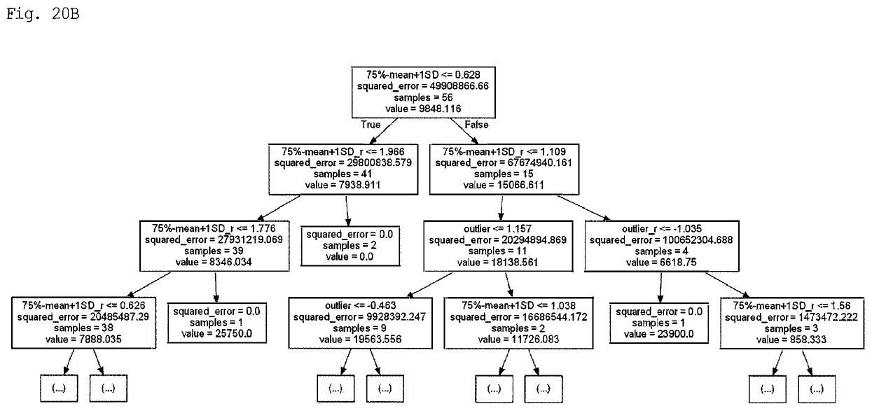

FIG. 15 shows the results of Example 1. In Example 1, a linear regression model was trained from all data as to predict the dyskinesia score in MPTP-induced PD-LID model rhesus monkeys from the blink duration parameter when only L-DOPA was administered (see FIG. 13). Using the trained model, the dyskinesia score was predicted using (B) the blink number parameter for each blink duration during administration of tandospirone as a feature.
(A) Predicted dyskinesia score (grey line) and true dyskinesia score (black line) are illustrated. A strong correlation was observed with the correlation coefficient r=0.84.

FIG. 16 shows the results of Example 3. In Example 3, a linear regression model was trained to predict L-DOPA concentration in plasma using the number of blinks for each blink duration in MPTP-induced PD-LID model rhesus monkeys as a feature. The training data and test data were split at 8:2.
(A) Plasma L-DOPA concentrations were measured every 30 minutes during L-DOPA administration in model monkeys and linearly approximated. Denote the blink duration as T(s) and the weighted parameter as θ.
(B) for T<0.14, θ=966, (C) for 0.14≤T<0.16, θ=−1310, (D) for 0.16≤T<0.18, θ=399, (E) For 0.18≤T<0.20, θ=3744, (F) For 0.20≤T<0.25, θ=759, and (G) for 0.25≤T, θ =−2448, respectively. The feature that best predicts the plasma L-DOPA concentration was the number of blinks of the blink duration that satisfies 0.18≤T<0.20.
(H) Predicted plasma L-DOPA concentrations were plotted on the vertical axis and true plasma L-DOPA concentrations on the horizontal axis. The correlation coefficient between the predicted value and the true value in the test data was r=0.83, indicating a strong correlation.

FIG. 17 is an example of a test flow for searching for objective parameters of ocular information correlated with Parkinson's symptoms.

FIG. 18A shows an example of a decision tree for constructing a random forest model constructed to predict dyskinesia scores in Example 1′.

FIG. 18B shows another example of decision trees for constructing a random forest model constructed to predict the dyskinesia score of Example 1′.

FIG. 18C shows the result of the prediction by the random forest model constructed in order to predict the dyskinesia score of Example 1′.

FIG. 19A shows an example of a decision tree for constructing a random forest model constructed to predict the generalized dyskinesia score of Example 1′.

FIG. 19B shows another example of a decision tree for constructing a random forest model constructed to predict the generalized dyskinesia score of Example 1′.

FIG. 19C shows the result of the prediction by the random forest model constructed in order to predict the generalized dyskinesia score of Example 1′.

FIG. 20A shows an example of the decision tree which constructs the random forest model constructed in order to predict L-DOPA concentration in peripheral blood of Example 3′.

FIG. 20B shows another example of a decision tree for constructing a random forest model constructed for predicting L-DOPA concentration in peripheral blood in Example 3′.

FIG. 20C shows the result of the prediction by the random forest model constructed in order to predict the L-DOPA concentration in peripheral blood of Example 3′.

### MODE FOR CARRYING OUT THE INVENTION

The present disclosure is described hereinafter. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. Further, the terms used herein should be understood as being used in the meaning that is commonly used in the art, unless specifically noted otherwise. Therefore, unless defined otherwise, all terminologies and scientific technical terms that are used herein have the same meaning as the general understanding of those skilled in the art to which the present disclosure pertains. In case of a contradiction, the present specification (including the definitions) takes precedence.

### Definition

Terminology and general techniques used in this disclosure are explained.

In the present specification, “about” means±10% of the numerical value that follows.

In the present specification, the term “subject” is used in the same sense as “test body” and “test person”, and refers to an organism that is the object of treatment, diagnosis, examination, or the like of the present disclosure, and when a subject is afflicted with a disease, it is used synonymously with “patient”. The subject includes any animal, but is preferably mammalian (rodents such as mice and rats, carnivora such as dogs, artiodactyls such as cows, pigs and goats, perissodactyla such as horses, or Primates), more preferably animals of the order Primates, and even more preferably humans.

In the present specification, “dopamine or a substance biologically equivalent to dopamine” refers to dopamine or any substance having a similar function to dopamine in vivo. One such function is its function as a neurotransmitter, and there are three main neural pathways where dopamine works: the nigrostriatal pathway, the mesolimbic pathway, and the mesolimbic pathway, and the striatal pathway is associated with Parkinson's disease, therefore, particularly in the present specification, if bioequivalence can be said in relation to the nigrostriatal pathway, it corresponds to a substance biologically equivalent to dopamine as defined in the present disclosure.

In the present specification, examples of substances biologically equivalent to dopamine include levodopa (L-3,4-dihydroxyphenylalanine (IUPAC name is (S)-2-amino-3-(3,4-dihydroxyphenyl) propanoic acid), also called L-dopa), and include additionally, but not limited to, L-3,4-dihydroxyphenylalanine, bromocriptine, pergolide, talipexole, cabergoline, pramipexole, ropinirole, rotigotine, apomorphine, and the like.

In the present specification, “L-DOPA or an L-DOPA-related compound or a dopamine agonist” refer to a substance that exerts a pharmacological effect of L-DOPA through in vivo metabolism when administered to a living body (for example, a patient with Parkinson's disease), for example, a substance that exerts a biological function of “dopamine or a substance biologically equivalent to dopamine”, and includes L-DOPA, and additionally, L-DOPA-related compounds, and dopamine agonists.

In the present specification, the term “dopamine agonist” includes, but is not limited to, L-3,4-dihydroxyphenylalanine, bromocriptine, pergolide, talipexole, cabergoline, pramipexole, ropinirole, rotigotine, apomorphine, and the like.

In the present specification, the term “L-DOPA” or “levodopa” are synonymous.

In the present disclosure, “L-DOPA-related compounds” are those that are different from L-DOPA but have equivalent biological functions to L-DOPA (e.g., activities related to the treatment of Parkinson's disease). For example, L-DOPA-related compounds include, but are not limited to, esters of L-3,4-dihydroxyphenylalanine and salts thereof. Examples of esters of L-3,4-dihydroxyphenylalanine include levodopa ethyl ester (LDEE; ethyl (2S)-2-amino-3-(3,4-dihydroxyphenyl) propanoate), levodopa propyl ester; levodopa propyl ester (propyl (2S)-2-amino-3-(3,4-dihydroxyphenyl)propanoate), levodopa methyl ester (methyl (2S)-2-amino-3-(3,4-dihydroxyphenyl)propanoate), etc. and the ester of L-3,4-dihydroxyphenylalanine can be, for example, a salt, including a hydrated salt. Levodopa ester salts can include, but are not limited to, any of octanoate, myristate, succinate, succinate dihydrate, fumarate, fumarate dihydrate, mesylate, tartrate and hydrochloride. For example, the succinate or succinate dihydrate of the ester of L-3,4-dihydroxyphenylalanine includes levodopa ethyl ester succinate (LDEE-S) or levodopa ethyl ester succinate dihydrate (LDEE-S-dihydrate or LDEE-S(d)). L-DOPA-related compounds also include L-DOPA prodrugs and depot preparations. L-DOPA-related compounds may overlap with dopamine agonists, but may be classified as either such case herein.

In the present specification, the term “adjunct” refers to a drug other than the drug that has the main action, and in the present disclosure, if levodopa is the main drug, tandospirone or the like can be the adjuvant. In the present specification, the term “L-DOPA adjunct” refers to a drug that suppresses the metabolism or degradation of L-DOPA or regulates the release of L-DOPA-derived dopamine into the synaptic cleft. In the present specification, the daily dose of levodopa, the main agent, is the usual dose for levodopa treatment described in the 2018 version of the Clinical Practice Guidelines for Parkinson's Disease or corresponding guidelines in the United States and Europe. Generally, the usual dose of levodopa per day is 50-1200 mg/day, preferably 100 mg-600 mg/day, in combination or as blended with a peripheral dopa decarboxylase inhibitor (DCI). For example, the FDA-approved SINEMET® (Carbidopa-Levodopa Combination Tablets) (New Drug Application (NDA) #017555) provides a 1:4 ratio combination tablet (Carbidopa 25 mg-Levodopa 100 mg), and a 1:10 ratio combination tablets (Carbidopa 10 mg-Levodopa 100 mg, Carbidopa 25 mg-Levodopa 250 mg). The daily maintenance dose of SINEMET® is 70 mg to 100 mg of Carbidopa, and the maximum daily dose of SINEMET® is 200 mg of Carbidopa.

Examples of levodopa auxiliary agents include levodopa metabolic enzyme inhibitors and dopamine release control agents.

In the present specification, the term “levodopa-metabolizing enzyme inhibitor” refers to any drug that inhibits the metabolism of levodopa so as to enhance the action of levodopa in a broad sense, and includes dopa decarboxylase (decarboxylase) inhibitors (DCIs) (carbidopa, alpha-methyldopa, benzelazide (Ro 4-4602), alpha-difluoromethyl-DOPA (DEMD) or salts thereof, etc.) that prevents the conversion of levodopa to dopamine in the intestine, liver and blood vessels; catecholamine-O-methyltransferase inhibitors (COMT-I) (exemplified by entacapone), which also prevent levodopa from being degraded before it enters the brain; and monoamine oxidase inhibitors (MAO-I) (exemplified by selegiline) that prevent dopamine from being broken down in the brain; and the like. The “dopamine release control agent” refers to a drug that has an effect of controlling dopamine release from dopamine neurons, and examples thereof include zonisamide, amantadine, tandospirone, buspirone, and istradefylline.

In the present specification, the term “dopamine neuronal function restorative agent” refers to any drug or therapeutic method for the purpose of restoring function of dopamine neuronsand/or preventing or suppressing degeneration loss, and includes substances having a dopaminergic neurodegenerative action, such as vaccines or antibody drugs against alpha-synuclein, or nucleic acid drugs that affect the expression thereof, and the like.

In the present specification, the term “dopamine producing cell therapy” refers to a drug that treats Parkinson's disease by cell transplantation of dopamine neurons. Examples include cell transplantation of fetal midbrain tissue and cell transplantation of human induced pluripotent stem cells (iPS cells)-derived dopaminergic progenitor cells.

In the present specification, “dopamine producing gene therapy” refers to a therapeutic method in which an enzyme gene required for dopamine synthesis is introduced into the putamen by a viral vector. An example is gene therapy in which an aromatic amino acid decarboxylase (AADC) gene is introduced into an adeno-associated virus (AAV).

In the present specification, “surgical therapy” includes surgical treatment methods such as Deep Brain Stimulation (DBS) to subthalamic nucleus or globus pallidus inner division or thalamus, stereotactic destruction of thalamus and globus pallidus, focused ultrasound therapy (Focused Ultrasound: FUS), etc.

In this disclosure, a reference to an “amount” may include an “absolute amount” in its narrow sense as well as a “relative amount” in its broad sense. A “relative amount” includes, for example, a change amount (e.g., an increase amount, a decrease amount).

In the present disclosure, “level” means an approximate degree, even if the amount cannot be specified. These levels refer to the degree of the effect (action), and can be expressed by clinical evaluation scales such as Movement Disorder Society-Unified Parkinson's Disease Rating Scale (MDS-UPDRS), UPDRS, Unified Dyskinesia Rating Scale (UDysRS), Clinical Dyskinesia Rating Scale (CDRS), The Rush Dyskinesia Rating Scale (Rush DRS), Abnormal Involuntary Movement Scale (AIMS), EuroQol 5 Dimensions (EQ-5D-5L), PDQ-39 (Parkinson's Disease Questionnaire-39), Clinical Global Impressions (CGI), Patient Global Impression (PGI), and the like; scales calculated from locomotion information obtained by wearable devices such as patient diaries, accelerometers and/or gyrometers; and the like.

In this disclosure, “variation” refers to change over time or a value that changes over time. A value that has changed over time is also referred to as an amount of change.

In the present disclosure, “in vivo dopamine or a substance biologically equivalent to dopamine” means dopamine a substance biologically equivalent to dopamine present in certain internal organs or organs (including blood, etc.), or cells.

In the present disclosure, “dopamine or a substance biologically equivalent to dopamine in the brain” refers to dopamine or a substance biologically equivalent to dopamine that is present in the brain. Dopamine in the brain or a substance biologically equivalent to dopamine can be measured by methods such as measurement of the amount of dopamine by a microdialysis method, mass spectrometry imaging (MSI), brain function imaging methods using single photon emission computed tomography (SPECT) formulations such as drugs undergoing metabolism similar to L-DOPA, drugs with affinity for dopamine transporters and dopamine receptors, and positron Emission Tomography (PET) formulations; and the like.

In the present disclosure, “ocular information” refers to information about the eyeball. Eyeball information includes, for example, information about eyeball movements. In the present disclosure, “eye movement” refers to general eye movement. Eye movements can include, for example, eye movements, eye condition changes, changes in electro-oculography, and the like. Even when the eyeball is stationary, it is included in the eye movement. Examples of ocular information include, for example, information on blinking, information on pupil coordinates or relative positions of the eyeball, and information on pupil diameter. Blinks include, for example, spontaneous blinks, voluntary blinks, and reflexive blinks. Examples of blink information include, but are not limited to, blink frequency or number of blinks, blink duration, time between blinks, eye closing time, eye opening speed, eye closing speed, eyelid movement width, and eyelid openness. For example, information on the pupillary coordinates or the relative position of the eyeball includes, but not limited to, the presence or absence, amount or level of movement distance, movement direction, velocity, acceleration, angular velocity, saccades, smooth eye movement, vestibulo-ocular reflex, convergence/divergence, fixational eye movement (ocular tremor, drift, microsaccades).

The information about the blink and the information about the pupil coordinates or the relative position of the eyeballs may also include calculated values of those information. The calculated value can include, for example, a function output (exponentiation, logarithm, distribution, frequency analysis value, etc.) with the information as an input variable. The calculated value can be, for example, the calculated value of the value in each division when the data is divided by a predetermined threshold (for example, the threshold for time (time window), the threshold for absolute value, the threshold for relative value) (calculated value within division). Intra-division calculated values include, but are not limited to, various statistics such as maximum value, minimum value, mean value, median value, variance, 25% percentile value, 75% percentile value, and frequency analysis spectrum. The calculated values may include, for example, calculated values of intra-partition calculated values of a plurality of partitions (inter-division calculated values). Inter-interval calculated values include, but are not limited to, weighted sums, differences, time differentiations, time integrals, ratios, correlation coefficients, covariances, for example.

In the present disclosure, “Parkinson's disease patients being treated with L-DOPA” refers to Parkinson's disease patients being treated with administration of L-DOPA, and the symptoms can be confirmed by clinical evaluation scales such as MDS-UPDRS, UPDRS, UDysRS, CDRS, Rush DRS, AIMS, EQ-5D-5L, PDQ-39, CGI, PGI, and the like, or scales calculated from locomotion information obtained by wearable devices such as patient diaries, accelerometers and/or gyrometers; and the like.

In the present disclosure, the term “therapeutic agent” refers to an agent for curing Parkinson's disease or relieving symptoms when referring to a Parkinson's disease patient. Clinically, an improvement in symptoms can be confirmed by clinical evaluation scales such as MDS-UPDRS, UPDRS, UDysRS, CDRS, RushDRS, AIMS, EQ-5D-5L, PDQ-39, CGI, PGI, etc. MDS-UPDRS, UPDRS, UDysRS, CDRS, RushDRS, AIMS, EQ-Clinical rating scales such as 5D-5L, PDQ-39, CGI, PGI, and the like, or scales calculated from locomotion information obtained by wearable devices such as patient diaries, accelerometers and/or gyrometers; and the like.

In the present disclosure, the term “prophylactic drug” refers to a drug for preventing worsening of Parkinson's disease or preventing symptoms (e.g., dyskinesia) from appearing when referring to Parkinson's disease patients. Clinically, the prophylactic effect can be confirmed by clinical evaluation scales such as MDS-UPDRS, UPDRS, UDysRS, CDRS, Rush DRS, AIMS, EQ-5D-5L, PDQ-39, CGI, PGI, and the like, or scales calculated from locomotion information obtained by wearable devices such as patient diaries, accelerometers and/or gyrometers; and the like.

In the present disclosure, “medical technology” refers to technology for taking some medical treatment, and can be provided in any form such as medicines, medical devices, regenerative medicine products, surgical therapy, and the like.

As used in this disclosure, “effective amount” or “effective level” refers to an amount or level effective for treating or preventing Parkinson's disease. In the present specification, the daily dose of levodopa, the main agent, is the usual dose for levodopa treatment described in the 2018 version of the Clinical Practice Guidelines for Parkinson's Disease or corresponding guidelines in the United States and Europe. Generally, the usual dose of levodopa per day is 50-1200 mg/day, preferably 100 mg-600 mg/day, in combination or blended with a peripheral dopa decarboxylase inhibitor (DCI). For example, the FDA-approved SINEMET® (Carbidopa-Levodopa Combination Tablets) (New Drug Application (NDA) #017555) is provided as a 1:4 ratio combination tablet (Carbidopa 25 mg-Levodopa 100 mg), and a 1:10 ratio combination tablets (Carbidopa 10 mg-Levodopa 100 mg, Carbidopa 25 mg-Levodopa 250 mg). The daily maintenance dose of SINEMET® is 70 mg to 100 mg of Carbidopa, and the maximum daily dose of SINEMET® is 200 mg of Carbidopa.

In the present disclosure, “the effect of a drug having an improving effect on L-DOPA or an L-DOPA-related compound or a dopamine agonist” refers to an effect of a medicine having an improving effect on L-DOPA or an L-DOPA-related compound or a dopamine agonist, and can be measured by various techniques as described below.

That is, the improving effect on levodopa-induced dyskinesia in Parkinson's disease in the present disclosure can be confirmed clinically by clinical evaluation scales such as MDS-UPDRS, UPDRS, UDysRS, CDRS, Rush DRS, AIMS, EQ-5D-5L, PDQ-39, CGI, PGI, and the like, or scales calculated from locomotion information obtained by wearable devices such as patient diaries, accelerometers and/or gyrometers; and the like. In non-clinical PD-LID model rats or PD-LID model monkeys, dyskinesia-improving effects can be confirmed by evaluating dyskinesia-like abnormal involuntary movement behavior. By using this method, it is possible to measure improvement, suppression or prevention of progression of levodopa-induced dyskinesia (PD-LID) symptoms, as well as shortening of levodopa-induced dyskinesia (PD-LID) onset time.

Parkinson's disease conditions measurable by the present disclosure include akinesia, bradykinesia, the main symptoms of motor symptoms, and tremor, muscle rigidity, loss of postural reflexes, floxed posture, freezing, sleep disorders, mental/cognitive/behavioral disorders, autonomic neuropathy, sensory disturbances, and any motor complications.

In the present specification, the term “motor complications” refers to any motor symptom that is problematic for treatment observed in patients with advanced Parkinson's disease, and includes dyskinesia (levodopa-induced dyskinesia (PD-LID)), which is involuntary movement associated with diurnal variation of Parkinson's symptoms and levodopa treatment; and the like. Motor complications have been interpreted as being based on abnormal release of dopamine in the brain, but the mechanism is not necessarily clear.

In the present specification, known “motor fluctuations of Parkinson's symptoms” include wearing-off, on-off phenomenon, no-on phenomenon, delayed-on (delayed on) phenomenon, and the like. Among them, wearing-off is a symptom in which as the disease progresses, the ability to retain dopamine in the synaptic cleft decreases, and the brain dopamine concentration fluctuates according to the blood concentration of levodopa, and consequently, wearing-off is seen in a shortened duration of effect of levodopa when blood concentrations fall below the safe therapeutic range.

In the present specification, “levodopa-induced dyskinesia<involuntary movement> (PD-LID)” is an involuntary movement (dyskinesia) induced by an overdose of levodopa, which occurs in more than half of Parkinson's disease patients five to ten years after starting treatment with levodopa, and percentage of patients affected by PD-LID is increasing over time (For reviews, see, for example, Encarnacion and Hauser, (2008), “Levodopa-induced dyskinesias in Parkinson's disease: etiology, impact on quality of life, and treatments.”), pages 57-66) Eur Neurol, 60(2).

Dyskinesia refers to involuntary movements of a part of the body that cannot be stopped, biting the lips, difficulty speaking, being unable to stay still, and difficulty moving the limbs as intended, and is movement disorder that results in involuntary movements of the limbs and/or orofacial and/or axial parts of the body. Peak-dose dyskinesia is known as a typical symptom of PD-LID, and symptoms appear on the face, tongue, neck, extremities, trunk, etc. when the levodopa blood concentration is high. Regarding PD-LID, it has been known that if you continue to take levodopa in large doses more than necessary from the early stage of the disease, it is likely to appear, and once PD-LID appears, it is very difficult to control even if you adjust the dose of levodopa in various ways.

### Explanation of Preferred Embodiment

Preferred embodiments of the present disclosure are described below. The embodiments provided below are provided for a better understanding of the disclosure, and it is understood that the scope of the disclosure should not be limited to the following description. Therefore, it is clear that a person skilled in the art can make appropriate modifications within the scope of the present disclosure in light of the description in the present specification. It is also understood that the following embodiments of the disclosure can be used singly or in combination.

It should be noted that the embodiments described below are all comprehensive or specific examples. Numerical values, shapes, materials, components, arrangement positions and connection forms of components, steps, order of steps, and the like shown in the following embodiments are examples, and are not intended to limit the scope of the claims. In addition, among the constituent elements in the following embodiments, constituent elements that are not described in independent claims representing the highest concept will be described as optional constituent elements.

### (Method for Predicting the Amount of Dopamine or a Substance Biologically Equivalent to Dopamine In Vivo)

In one aspect of the present disclosure, provided are a method of predicting the amount of in vivo dopamine or a substance biologically equivalent to dopamine based on ocular information in a subject, a computer program for realizing this or a recording medium storing this, a user equipment that constitutes a system or part of that system; and the like. In this aspect, the present disclosure provides a method of predicting the presence or absence, amount or level of in vivo dopamine or a substance biologically equivalent to dopamine in the subject, or a variation thereof. The in vivo dopamine or the substance biologically equivalent to dopamine can include any part of a living body, but particularly can include dopamine in the brain or a substance biologically equivalent to dopamine. While not wishing to be bound by theory, conventionally, the concentration of in vivo dopamine or a substance biologically equivalent to dopamine was thought to be related to involuntary movements, but it is not known that it is accurately linked to eye movements, and by examining ocular information more accurately and in real time according to the present disclosure, it is possible to know the concentration of dopamine or a substance biologically equivalent to dopamine in the body in real time.

L-DOPA is used as a standard treatment for Parkinson's disease, and in current treatments, there is some interest in whether it is possible to control and manage the amount of L-DOPA or similar dopamine agonists (and dopamine as a converted substance or a substance biologically equivalent to dopamine) in the body (especially in the brain, which is the site of action). The present disclosure can know the level of dopamine or a substance biologically equivalent to dopamine in the living body from eye information without examining the inside of the living body, and this makes it possible to obtain an indication for the status, therapeutic efficacy and/or further treatment of Parkinson's disease patients being treated with dopamine or a substance biologically equivalent to dopamine. Controlling the in vivo (brain) concentration of dopamine or a substance biologically equivalent to dopamine through an appropriate dosage and administration method of L-DOPA or an L-DOPA-related compound or a dopamine agonist (for example, dopamine agonists exhibiting activity similar to that of dopamine) possibility can achieve both improvement of Parkinson's symptoms and prevention of motor complications.

In the present disclosure, by performing an unprecedented detailed analysis of eye movement parameters including eye blinking, parameters that are thought to reflect dopamine variations more sensitively than spontaneous blink frequency were found.

Since the present disclosure has shown that the amount of dopamine or a substance biologically equivalent to dopamine in vivo, including those in the brain, tends to correlate with the number of blinks, for example, the correlation between the number of blinks and the amount of dopamine in healthy subjects can be used as an indicator to measure the number of blinks in patients with Parkinson's disease, and the amount of dopamine can be predicted from this measured value, in the present disclosure. In other embodiments, the presence or absence, amount or level of L-DOPA, or a variation thereof can be predicted, by previously obtaining a correlation between an increase in the blink number for every blink duration (e.g., >0.25 s, 0.25 s, 0.20 s, 0.16 s, 0.14 s, <0.12 seconds, etc.) when L-DOPA is administered to Parkinson's disease patients and development of Parkinson's symptoms, motor complications, psychiatric symptoms, or an increase or decrease in the amount of dopamine or substances with dopamine-like biological functions, and blood L-DOPA concentration, a variation, etc., and by applying the ocular information obtained from the target patient.

A specific calculation method for in vivo dopamine or a substance biologically equivalent to dopamine is as follows.

(Method for Estimating Dopamine or a Substance Biologically Equivalent to Dopamine in Parkinson's Disease Patients being Treated with L-DOPA or an L-DOPA-Related Compound or a Dopamine Agonist)

In one embodiment, for a Parkinson's disease patient, the ocular information is measured by devices capable of acquiring ocular information, for example, an image recording function, an eye tracking function, a spectacle-type device equipped with a three-point electrooculogram sensor, an acceleration sensor, and/or glasses type device with gyro sensor, an eye tracking glass, a smartphone, a personal computer, an electrooculogram measuring device, etc. Eyeball information includes, for example, information on blinking, information on pupil coordinates or relative positions of eyeballs, and information on eyeball movements. Blinks include, for example, voluntary blinks, voluntary blinks, and reflexive blinks. Examples of blink information include, but are not limited to, blink frequency or number of blinks, blink duration, time between blinks, eye closing time, eye opening speed, eye closing speed, eyelid movement width, and eyelid openness. For example, information related to eye movement includes, for example, parameters such as eye movement distance, eye movement direction, eye movement speed, eye acceleration, eye angular velocity, saccade, smooth eye movement, vestibulo-ocular reflex, convergence/divergence, fixational eye movement (ocular tremor, drift, microsaccades), pupil diameter, and the like, or function output (power, logarithm, distribution, frequency analysis value, etc.) with these information as input variables. The calculated value can be, for example, the calculated value of the value in each division when the data is divided by a predetermined threshold (for example, the threshold for time (time window), the threshold for absolute value, the threshold for relative value) (calculated value within division). Intra-division calculated values include, but are not limited to, various statistics such as maximum value, minimum value, mean value, median value, variance, 25% percentile value, 75% percentile value, and frequency analysis spectrum. The calculated values may include, for example, calculated values of intra-partition calculated values of a plurality of partitions (inter-division calculated values). For the inter-division calculated values, for example, calculated values such as weighted sum, difference, time differentiation, time integration, ratio, correlation coefficient, and covariance can be obtained.

The measurement time per measurement is determined to be long enough to relatively stably measure ocular information, and may range from 30 seconds to 16 hours per measurement. More preferably, it ranges from 5 minutes to 8 hours. During the measurement, the patient may be engaged in an unrestricted daily routine or may be performing a task to measure specific eye movements. Examples of tasks include tracking a marker that suddenly disappears and appears on a display as a visual stimulus to induce saccades, and tracking a marker that moves smoothly to induce smooth eye movements, and the like. In this case, an accelerometer or the like attached to the arm or head may be used in combination to evaluate ocular information in conjunction with movement of the head or arm, alternatively, devices for immobilizing the head and jaw may be used in combination to eliminate the motion elements of the head. Patients may also complete a patient diary to record subjective symptoms.

In one embodiment, a time interval or “time window” of digital data is extracted from the time series data. The duration of each time window can be set so that it is long enough so that each ocular data measurement and the amount of in vivo dopamine or a substance biologically equivalent to dopamine can be relatively stably determined, but is short enough to capture time-varying amounts of dopamine or a substance biologically equivalent to dopamine in vivo. For example, the width of the time window ranges from 10 seconds to 30 minutes, more preferably from 1 minute to 10 minutes.

In one embodiment, data from an eye tracking device is filtered to remove noise as well as extract data of interest. For the blink data, for example, abnormally long blink events can be filtered out, such as are not generally possible with human blinks, e.g., blink durations greater than 1000 milliseconds, but if use by users with abnormal blinking, such as Parkinson's disease patients, is envisaged, the exclusion criterion can be relaxed, for example 2000 milliseconds. For eye movement data, for example, when focusing on eye tremor, eye movement with a large distance such as saccades becomes noise that interferes with frequency analysis, therefore, time window data containing data points with large moving distances can be preliminarily excluded.

In some embodiments, eye movement is obtained as discrete pupil coordinate data, but the data points may be smoothly interpolated with a spline curve or the like and upsampled to obtain sufficient temporal resolution.

In some embodiments, the user's eye measurements may also be normalized or corrected based on the user's past measurements or measurements taken by other users.

In another embodiment, the amount of in vivo dopamine or a substance biologically equivalent to dopamine can be predicted from the correlation between ocular information and the amount of in vivo dopamine or a substance biologically equivalent to dopamine. The amount of dopamine or a substance biologically equivalent to dopamine in vivo can be predicted from the correlation between the aforementioned eye movement parameter and the amount of dopamine or a substance biologically equivalent to dopamine in vivo. Specifically, by inputting appropriately selected parameters among these parameters into the trained model as features, it is possible to estimate the amount of dopamine or a substance biologically equivalent to dopamine in vivo at present or after a certain period of time.

In one embodiment, when estimating the amount or level of dopamine or a substance biologically equivalent to dopamine in peripheral blood, blink frequency information in which the blink duration is within a certain range can be used. The certain range is, for example, a range from 0.18 s to 0.25 s in absolute value. This range may be corrected or normalized based on individual patient variability and day-to-day variability, and may be corrected or normalized based on historical patient population data. In addition, the blink time may be divided into fixed time intervals (for example, 0.02 s) and weighted independently according to the strength of the correlation. The resulting weighted sum of the number and/or increments in a given time window can be used as an estimate of the concentration or level of dopamine or a substance biologically equivalent to dopamine in peripheral blood.

In one embodiment, blink frequency information with a certain range of blink durations can be used when estimating variations in brain dopamine or a substance biologically equivalent to dopamine. The certain range is, for example, a range from 0.18 s to 0.25 s in absolute value. This range may be corrected or normalized based on individual patient variability and day-to-day variability, and may be corrected or normalized based on historical patient population data. In addition, the blink time may be divided into fixed time intervals (for example, 0.02 s) and weighted independently according to the strength of the correlation. The resulting weighted sum of the number and/or increments in a given time window can be used as an estimate of brain dopamine concentration or level.

In one aspect of the present disclosure, a method of estimating the amount or level of in vivo dopamine or a substance biologically equivalent to dopamine in a Parkinson's disease patient being treated with L-DOPA or an L-DOPA-related compound or a dopamine agonist, a computer program for realizing this or a recording medium storing it, a system or a user device constituting a part of the system, and the like are provided. This method includes the steps of: A) obtaining ocular information of the patient; and B) calculating the estimated presence or absence, amount or level of dopamine or a substance biologically equivalent to dopamine in the patient based on the ocular information.

In other embodiments, the estimating step may estimate changes in dopamine amounts in the synaptic cleft of the striatum upon administration of L-DOPA.

(Method for Estimating the Amount of L-DOPA or an L-DOPA-Related Compound or a Dopamine Agonist In Vivo in Parkinson's Disease Patients being Treated with L-DOPA or an L-DOPA-Related Compound or a Dopamine Agonist)

In one embodiment, for a Parkinson's disease patient, the ocular information is measured by devices capable of acquiring ocular information, for example, an image recording function, an eye tracking function, a spectacle-type device equipped with a three-point electrooculogram sensor, an acceleration sensor, and/or glasses type device with gyro sensor, an eye tracking glass, a smartphone, a personal computer, an electrooculogram measuring device, etc. Eyeball information includes, for example, information on blinking, information on pupil coordinates or relative positions of eyeballs, and information on eyeball movements. Blinks include, for example, voluntary blinks, voluntary blinks, and reflexive blinks. Examples of blink information include, but are not limited to, blink frequency or number of blinks, blink duration, time between blinks, eye closing time, eye opening speed, eye closing speed, eyelid movement width, and eyelid openness. For example, eye movement information can include parameters such as eye movement distance, eye movement direction, eye movement speed, eye acceleration, eye angular velocity, saccade, smooth eye movement, vestibulo-ocular reflex, convergence/divergence, fixational eye movement (ocular tremor, drift, microsaccades), pupillary diameter, or function output (power, logarithm, distribution, frequency analysis value, etc.) with these information as input variables. The calculated value can include, for example, the calculated value of the value in each division when the data is divided by a predetermined threshold (for example, the threshold for time (time window), the threshold for absolute value, the threshold for relative value) (calculated value within division). Intra-division calculated values include, but are not limited to, various statistics such as maximum value, minimum value, mean value, median value, variance, 25% percentile value, 75% percentile value, and frequency analysis spectrum. The calculated values may include, for example, calculated values of intra-partition calculated values of a plurality of partitions (inter-division calculated values). As inter-division calculated values, for example, calculated values such as weighted sums, differences, time differentials, time integrals, ratios, correlation coefficients, covariances, and the like can be obtained.

The measurement time per measurement is determined to be long enough to relatively stably measure ocular information, and may range from 30 seconds to 16 hours per measurement. More preferably, it ranges from 5 minutes to 8 hours. During the measurement, the patient may be engaged in an unrestricted daily routine or may be performing a task to measure specific eye movements. Examples of tasks include tracking a marker that suddenly disappears and appears on a display as a visual stimulus to induce saccades, tracking a marker that moves smoothly to induce smooth eye movements, and the like. In this case, an accelerometer or the like attached to the arm or head may be used in combination to evaluate ocular information in conjunction with movement of the head or arm, alternatively devices for immobilizing the head and jaw may be used in combination to eliminate the motion elements of the head. Patients may also complete a patient diary to record subjective symptoms.

In one embodiment, time intervals or “time windows” of digital data are extracted from the time-series data, and the duration of each time window is determined so as to be sufficiently long so that the measured value of each ocular data and the amount of in vivo dopamine or a substance biologically equivalent to dopamine can be determined relatively stable, while it is set so as to be sufficiently short so that changes over time in the amount of in vivo dopamine or a substance biologically equivalent to dopamine are captured. For example, the width of the time window ranges from 10 seconds to 30 minutes, more preferably from 1 minute to 10 minutes.

In one embodiment, data from an eye tracking device is filtered to remove noise as well as extract data of interest. For the blink data, for example, abnormally long blink events can be filtered out, such as are not generally possible with human blinks, e.g., blink durations greater than 1000 milliseconds, but if use by users with abnormal blinking, such as Parkinson's disease patients, is envisaged, the exclusion criterion can be relaxed, for example 2000 milliseconds. For eye movement data, for example, when focusing on eye tremor, eye movement with a large distance such as saccades becomes noise that interferes with frequency analysis, therefore, time window data containing data points with large moving distances can be preliminarily excluded.

In some embodiments, eye movement is obtained as discrete pupil coordinate data, but the data points may be smoothly interpolated with a spline curve or the like and upsampled to obtain sufficient temporal resolution.

In some embodiments, the user's eye measurements may also be normalized or corrected based on the user's past measurements or measurements taken by other users.

In another embodiment, the amount of L-DOPA or an L-DOPA-related compound or a dopamine agonist in vivo can be predicted from the correlation between the ocular information and the amount of L-DOPA or an L-DOPA-related compound or a dopamine agonist in vivo. The amount of in vivo L-DOPA or an L-DOPA-related compound or a dopamine agonist can be predicted from the correlation between the aforementioned eye movement parameter and the in vivo L-DOPA or an L-DOPA-related compound or a dopamine agonist amount. Specifically, by inputting appropriately selected parameters among these parameters into the trained model as features, it is possible to estimate in vivo L-DOPA or an L-DOPA-related compound or a dopamine agonist at present or after a certain period of time.

In one embodiment, when estimating the amount or level of L-DOPA or an L-DOPA-related compound or a dopamine agonist in peripheral blood, blink frequency information with a certain range of blink duration can be used. The certain range is, for example, a range from 0.18 s to 0.25 s in absolute value. This range may be corrected or normalized based on individual patient variability and day-to-day variability, and may be corrected or normalized based on historical patient population data. Alternatively, the blink time may be divided into fixed time intervals (for example, 0.02 s) and weighted independently according to the strength of the correlation. The resulting weighted sum of times and/or increments over a given time window can be used as an estimate of the concentrations or level of L-DOPA or an L-DOPA-related compound or a dopamine agonist in peripheral blood.

In one aspect of the present disclosure, a method of estimating the amount of L-DOPA or an L-DOPA-related compound or a dopamine agonist in vivo in a Parkinson's disease patient being treated with L-DOPA or an L-DOPA-related compound or a dopamine agonist, a computer program for realizing this or a recording medium storing it, a system or a user device constituting a part of the system, and the like are provided. This method comprises the steps of: A) acquiring ocular information of the patient; and B) calculating the estimated presence or absence, amount or level of L-DOPA or an L-DOPA-related compound or a dopamine agonist in vivo in the patient based on the ocular information.

As the patient's in vivo L-DOPA or an L-DOPA-related compound or a dopamine agonist, preferably intracerebral L-DOPA or an L-DOPA-related compound or a dopamine agonist can be used. L-DOPA or an L-DOPA-related compound or a dopamine agonist can include, for example, L-DOPA. Symptoms of Parkinson's disease patients vary preferably depending on the extent of the amount or level or the presence or absence of these calculated factors, and for example, in the case of L-DOPA, the amount indicates whether the patient is on the treatment condition. In addition, depending on the progress of the disease, a correlation between an increase in the concentration of L-DOPA or an L-DOPA-related compound or a dopamine agonist taken into the body and the level of dopamine or a substance biologically equivalent to dopamine and associated treatment condition can also be found.

(Methods for Evaluating Therapeutic or Prophylactic Agent or Other Medical Technology for Parkinson's Disease Patients being Treated with L-DOPA or an L-DOPA-Related Compound or a Dopamine Agonist)

In one embodiment, for a Parkinson's disease patient, the ocular information is measured by devices capable of acquiring ocular information, for example, an image recording function, an eye tracking function, a spectacle-type device equipped with a three-point electrooculogram sensor, an acceleration sensor, and/or glasses type device with gyro sensor, an eye tracking glass, a smartphone, a personal computer, an electrooculogram measuring device, etc. Eyeball information includes, for example, information on blinking, information on pupil coordinates or relative positions of eyeballs, and information on eyeball movements. Blinks include, for example, spontaneous blinks, voluntary blinks, and reflexive blinks. Examples of blink information include, but are not limited to, blink frequency or number of blinks, blink duration, time between blinks, eye closing time, eye opening speed, eye closing speed, eyelid movement width, and eyelid openness. For example, eye movement information can include parameters such as eye movement distance, eye movement direction, eye movement speed, eye acceleration, eye angular velocity, saccade, smooth eye movement, vestibulo-ocular reflex, convergence/divergence, fixational eye movement (ocular tremor, drift, microsaccades), pupillary diameter, or function output (power, logarithm, distribution, frequency analysis value, etc.) with these information as input variables. The calculated value can include, for example, the calculated value of the value in each division when the data is divided by a predetermined threshold (for example, the threshold for time (time window), the threshold for absolute value, the threshold for relative value) (calculated value within division). Intra-division calculated values include, but are not limited to, various statistics such as maximum value, minimum value, mean value, median value, dispersion, 25% percentile value, 75% percentile value, frequency analysis spectrum, and the like. The calculated values may include, for example, calculated values of intra-partition calculated values of a plurality of partitions (inter-division calculated values). As inter-division calculated values, for example, calculated values such as weighted sums, differences, time differentials, time integrals, ratios, correlation coefficients, covariances, and the like can be obtained.

The measurement time per measurement is determined to be long enough to relatively stably measure ocular information, and may range from 30 seconds to 16 hours per measurement. More preferably, it ranges from 5 minutes to 8 hours. During the measurement, the patient may be engaged in an unrestricted daily routine or may be performing a task to measure specific eye movements. Examples of tasks include tracking a marker that suddenly disappears and appears on a display as a visual stimulus to induce saccades, tracking a marker that moves smoothly to induce smooth eye movements, and the like. In this case, an accelerometer or the like attached to the arm or head may be used in combination to evaluate ocular information in conjunction with movement of the head or arm, alternatively devices for immobilizing the head and jaw may be used in combination to eliminate the motion elements of the head. Patients may also complete a patient diary to record subjective symptoms.

In some embodiments, time intervals or “time windows” of digital data can be extracted from time-series data. The duration of each time window is set so as to be sufficiently long so that the measured value of each ocular data and the condition of a Parkinson's disease patient being treated with L-DOPA or an L-DOPA-related compound or a dopamine agonist are determined relatively stable, while it is set so as to be sufficiently short so that the time change of the condition of a Parkinson's disease patient being treated with L-DOPA or an L-DOPA-related compound or a dopamine agonist is captured. For example, the width of the time window ranges from 10 seconds to 30 minutes, more preferably from 1 minute to 10 minutes.

In one embodiment, data from an eye tracking device is filtered to remove noise as well as extract data of interest. For the blink data, for example, abnormally long blink events can be filtered out, such as are not generally possible with human blinks, e.g., blink durations greater than 1000 milliseconds, but if use by users with abnormal blinking, such as Parkinson's disease patients, is envisaged, the exclusion criterion can be relaxed, for example 2000 milliseconds. For eye movement data, for example, when focusing on eye tremor, eye movement with a large distance such as saccades becomes noise that interferes with frequency analysis, therefore, time window data containing data points with large moving distances can be preliminarily excluded.

In some embodiments, eye movement is obtained as discrete pupil coordinate data, but the data points may be smoothly interpolated with a spline curve or the like and upsampled to obtain sufficient temporal resolution.

In some embodiments, the user's eye measurements may also be normalized or corrected based on the user's past measurements or measurements taken by other users.

In addition in certain embodiments, the condition of a Parkinson's disease patient being treated with L-DOPA or an L-DOPA-related compound or a dopamine agonist can be predicted from a correlation between ocular information and clinical evaluation scales such as MDS-UPDRS, UPDRS, UDysRS, CDRS, Rush DRS, AIMS, EQ-5D-5L, PDQ-39, CGI, PGI, and the like, or an output from patient diaries or other wearable devices for evaluation of the condition of Parkinson's disease patients, and the like. By inputting appropriately selected parameters among the ocular movement parameters into the trained model as features, it is possible to estimate the condition of Parkinson's disease patients being treated with L-DOPA or an L-DOPA-related compound or a dopamine agonist at present or after a certain period of time, and to evaluate the therapeutic or prophylactic agent or other medical technology.

In one embodiment, by obtaining ocular information at a range of times before and after the patient has taken a new therapeutic, prophylactic, or other medical technology, respectively and comparing the conditions of the patient estimated therefrom, it is possible to evaluate the efficacy of the therapeutic, prophylactic, or other medical technology. A time range is, for example, 10 minutes to 1 year, more preferably 30 minutes to 1 month.

In addition, in certain embodiments, ocular information in Parkinson's disease patients being treated with L-DOPA or an L-DOPA-related compound or a dopamine agonist can be used to evaluate adequacy of current therapy and optimize therapy. For example, blink parameters are thought to be correlated with symptoms such as dopamine release and dyskinesias, so it is possible to infer from ocular information that dopamine is released in the striatum at excessive levels beyond the range appropriate for treatment. In such patients, dyskinesia is expected to be improved by suppressing dopamine release to a favorable level, for example, with an adjuvant that suppresses dopamine release (e.g., tandospirone), physicians can be assisted in selecting the most appropriate therapeutic or preventive agents or other medical technology for the patient.

Further, in certain embodiments, by previously examining the correlation between the dose or treatment dose of a therapeutic or prophylactic agent or other medical technology in a Parkinson's disease patient and changes in the Parkinson's disease patient's condition, optimal doses or treatment dose of the therapeutic or prophylactic drugs or other medical technology can also be estimated from eye movement information.

In one aspect of the present disclosure, methods for evaluating a therapeutic or prophylactic agent or other medical technology for Parkinson's disease patients being treated with L-DOPA or an L-DOPA-related compound or a dopamine agonist, and computer programs for implementing the same, a recording medium storing the same, a system or a user device constituting a part of the system, etc. are provided. This method includes A) a step of obtaining ocular information of the patient; B) a step of calculating the estimated presence or absence, amount or level of L-DOPA or an L-DOPA-related compound in vivo in the patient based on the ocular information.; C) a step of estimating or predicting the patient's condition based on the ocular information; and D) a step of calculating an estimated effective amount or effective level of the therapeutic or prophylactic agent or other medical technology from the presence/absence, amount or level, or a variation thereof, and the patient's condition.

In one embodiment, the therapeutic or prophylactic agent or other medical technology can be L-DOPA or an L-DOPA-related compound, an L-DOPA adjunct, a dopamine neuronal function restoring agent, a dopamine producing cell medicine, or a dopamine producing gene therapy or a surgical therapy (deep brain stimulation therapy, stereotactic ablation, etc.).

Thus, in a preferred embodiment, in the present disclosure, the method of the present disclosure can further comprise E) a step of calculating an estimated effective amount or effective level of L-DOPA or an L-DOPA-related compound or a dopamine agonist, an L-DOPA adjunct, a dopamine neuronal function restoring agent, a dopamine producing cell medicine or a dopamine producing gene therapy in the patient based on the estimated effective amount or effective level.

In one embodiment, the effect of the medicine may include suppression of flactuation and decreases over time in the amount of dopamine in the synaptic cleft of the striatum or a dopamine agonist when L-DOPA or an L-DOPA-related compound or a dopamine agonist is administered.

In one embodiment, blink frequency information can be used when estimating ON/OFF as the condition of a Parkinson's disease patient. Since the frequency of blinking decreases when the switch is ON, it can be regarded as ON when the frequency of blinking exceeds a certain threshold. The threshold is set, for example, in the range of 10 to 30 times per minute. This threshold may be corrected or normalized based on individual patient variability, day-to-day variability, and may be corrected or normalized based on historical patient population data.

In one embodiment, when estimating ON/OFF as the condition of a Parkinson's disease patient, a ratio to the weighted sum (short blink weighted sum/long blink weighted sum) for times per time window and/or increments can be used, in short blinks with blink durations below a certain level and long blinks above a certain level, based on the blink duration. A moderate or higher ratio (e.g., 1.5 or higher) can be an indicator for dyskinesia, but an appropriate lower range (e.g., 0.2 to 1.5) can be an indicator of ON which improves motor symptoms without dyskinesia. The indicator range may be corrected or normalized based on individual patient variability and day-to-day variability, and may be corrected or standardized based on historical patient population data.

By estimating the patient's condition in this way, it is possible to predict the therapeutic effect and improving effect of the drug administered to the patient who is the subject of treatment. For example, it is possible to estimate whether rapid changes or decrease over time in the amount of dopamine or a dopamine agonist in the synaptic cleft of the striatum during administration of L-DOPA or an L-DOPA-related compound or a dopamine agonist can be suppressed or not, and such information can be used to suppress the development of Parkinson's symptoms, motor complications, and psychiatric symptoms in patients.

(Method for Predicting the Presence or Absence, Severity, Score, Etc. Of Dyskinesia)

In one embodiment, for a Parkinson's disease patient, the ocular information is measured by devices capable of acquiring ocular information, for example, an image recording function, an eye tracking function, a spectacle-type device equipped with a three-point electrooculogram sensor, an acceleration sensor, and/or glasses type device with gyro sensor, an eye tracking glass, a smartphone, a personal computer, an electrooculogram measuring device, etc. Eyeball information includes, for example, information on blinking, information on pupil coordinates or relative positions of eyeballs, and information on eyeball movements. Blinks include, for example, spontaneous blinks, voluntary blinks, and reflexive blinks. Examples of blink information include, but are not limited to, blink frequency or number of blinks, blink duration, time between blinks, eye closing time, eye opening speed, eye closing speed, eyelid movement width, and eyelid openness. For example, eye movement information can include parameters such as eye movement distance, eye movement direction, eye movement speed, eye acceleration, eye angular velocity, saccade, smooth eye movement, vestibulo-ocular reflex, convergence/divergence, fixational eye movement (ocular tremor, drift, microsaccades), pupillary diameter, or function output (power, logarithm, distribution, frequency analysis value, etc.) with these information as input variables. The calculated value can include, for example, the calculated value of the value in each division when the data is divided by a predetermined threshold (for example, the threshold for time (time window), the threshold for absolute value, the threshold for relative value) (calculated value within division). Intra-division calculated values include, but are not limited to, various statistics such as maximum value, minimum value, mean value, median value, dispersion, 25% percentile value, 75% percentile value, frequency analysis spectrum, and the like. The calculated values may include, for example, calculated values of intra-partition calculated values of a plurality of partitions (inter-division calculated values). As inter-division calculated values, for example, calculated values such as weighted sums, differences, time differentials, time integrals, ratios, correlation coefficients, covariances, and the like can be obtained.

The measurement time per measurement is determined to be long enough to relatively stably measure ocular information, and may range from 30 seconds to 16 hours per measurement. More preferably, it ranges from 5 minutes to 8 hours. During the measurement, the patient may be engaged in an unrestricted daily routine or may be performing a task to measure specific eye movements. Examples of tasks include tracking a marker that suddenly disappears and appears on a display as a visual stimulus to induce saccades, and tracking a marker that moves smoothly to induce smooth eye movements. In this case, an accelerometer or the like attached to the arm or head may be used together in order to evaluate the ocular information in conjunction with the movement of the head or arm, or equipment for fixing the head and jaw may be used together to remove the movement element of the head. Patients may also complete a patient diary to record subjective symptoms.

In some embodiments, time intervals or “time windows” of digital data can be extracted from time-series data. The duration of each time window is long enough to relatively stably determine the measured value of each ocular data and the presence or absence, severity, score, etc. of dyskinesias in patients with Parkinson's disease, but is set to be short enough to capture temporal change of the presence or absence, severity, score, etc. For example, the width of the time window ranges from 10 seconds to 30 minutes, more preferably from 1 minute to 10 minutes.

In one embodiment, data from an eye tracking device is filtered to remove noise as well as extract data of interest. For the blink data, for example, abnormally long blink events can be filtered out, such as are not generally possible with human blinks, e.g., blink durations greater than 1000 milliseconds, but if use by users with abnormal blinking, such as Parkinson's disease patients, is envisaged, the exclusion criterion can be relaxed, for example 2000 milliseconds. For eye movement data, for example, when focusing on eye tremor, eye movement with a large distance such as saccades becomes noise that interferes with frequency analysis, therefore, time window data containing data points with large moving distances can be preliminarily excluded.

In some embodiments, eye movement is obtained as discrete pupil coordinate data, but the data points may be smoothly interpolated with a spline curve or the like and upsampled to obtain sufficient temporal resolution.

In some embodiments, the user's eye measurements may also be normalized or corrected based on the user's past measurements or measurements taken by other users.

In addition, in certain embodiments, specific methods for estimating dyskinesias in patients with Parkinson's disease can be clinically predicted from a correlation between clinical evaluation scales such as MDS-UPDRS, UPDRS, UDysRS, CDRS, Rush DRS, AIMS, EQ-5D-5L5D, PDQ-39, CGI, PGI, and the like, patient diaries, outputs from other wearable devices intended for evaluating dyskinesias in Parkinson's disease patients, etc., and the aforementioned eye movement parameters. Specifically, by inputting appropriately selected parameters among these parameters into a trained model as features, it is possible to estimate the presence or absence, severity, score, etc. of dyskinesias in Parkinson's disease patients at present or after a certain period of time.

In one embodiment, when predicting in advance the occurrence of dyskinesia after taking L-DOPA, L-DOPA-related compounds, or dopamine agonists, it is possible to use the number per the time window of “long blinks” where the blink duration is more than a certain level or the amount of change thereof. In the present disclosure, it has been found that long eye blinks are sharply increased by 15 to 45 minutes prior to the onset of dyskinesias following administration of L-DOPA or an L-DOPA-related compound or a dopamine agonist. The long blink duration, in absolute value, can range, for example, from 0.16 s to 0.50 s, more preferably from 0.18 s to 0.25 s. This range may be corrected or normalized based on individual patient variability and day-to-day variability, and may be corrected or normalized based on historical patient population data. In addition, long blink times may be divided into fixed time intervals (e.g., 0.02 s) and independently weighted according to the strength of the correlation. The weighted sum of the number and/or increments of long blinks in a given time window thus obtained can be used as an indicator of the probability of dyskinesia occurrence after a given period of time, e.g., 10 minutes.

In one embodiment, when estimating the presence or absence and severity of dyskinesia at the time of measurement, the number of “short blinks” in which the blink duration is less than a certain value can be used. In the present disclosure, it has been found that short blinks correlate with the presence or absence and severity of dyskinesias. A short blink duration may be less than 0.18 s in absolute value, for example. This range may be corrected or normalized based on individual patient variability and day-to-day variability, and may be corrected or normalized based on historical patient population data. In addition, the short blink time may be divided into fixed time intervals (for example, 0.02 s) and weighted independently according to the strength of the correlation. The resulting weighted sum of the number and/or increments in the short blink time window can be a probabilistic indicator of the presence and severity of dyskinesias.

In one embodiment, when estimating the severity of dyskinesia at the time of measurement, it is possible to use a ratio to the weighted sum (short blink weighted sum/long blink weighted sum) for the number and/or increments per time window, in short blinks with a blink duration of less than a certain amount and long blinks with a blink duration of more than a certain amount, based on the blink duration. This ratio is an indicator that is positively correlated with the occurrence of severe dyskinesias, and a moderate number (e.g., 1.5 or more) is considered to have dyskinesia, and a high value (e.g., 3.0 or more) can be assumed that a very strong dyskinesia has occurred.

In one embodiment, information about eye movements can be used when estimating the severity of dyskinesias at the time of measurement. Because ocular tremors increase with dyskinesia severity, frequency analysis of pupillary coordinates, eye movement distance, and eye movement velocity produces characteristic spectral bands whose power correlates with severity.

In one embodiment, eyelid opening information can be used when estimating the severity of dyskinesias at the time of measurement. If dyskinesias are severe, the eyelids may droop and vibrate erratically. Thus, for example, dyskinesia severity can be estimated from a decrease in eyelid opening and an increase in its variance.

In one aspect of the present disclosure, a method for predicting the presence or absence, severity, and score of dyskinesia based on ocular information, a computer program for realizing the same, a recording medium storing the same, and a system or a user device constituting a part of the system, and the like are provided. This method includes A) a step of obtaining ocular information of the patient; B) a step of calculating the estimated presence or absence, amount or level of L-DOPA or an L-DOPA-related compound in vivo in the patient based on the ocular information; and C) a step of estimating or predicting the presence or absence or severity or score of dyskinesia in the patient based on the ocular information.

### (System for Predicting the Presence or Absence, Amount or Level of In Vivo Dopamine or a Substance Biologically Equivalent to Dopamine in a Target, or its Variation)

The method of predicting the presence or absence, amount or level of in vivo dopamine or a substance biologically equivalent to dopamine in the subject, or a variation thereof, is performed using the system 10 of the present disclosure, for example.

The system 10 is configured to calculate the presence or absence, amount or level of in vivo dopamine or a substance biologically equivalent to dopamine in a subject, or a variation thereof, based on the subject's ocular information.

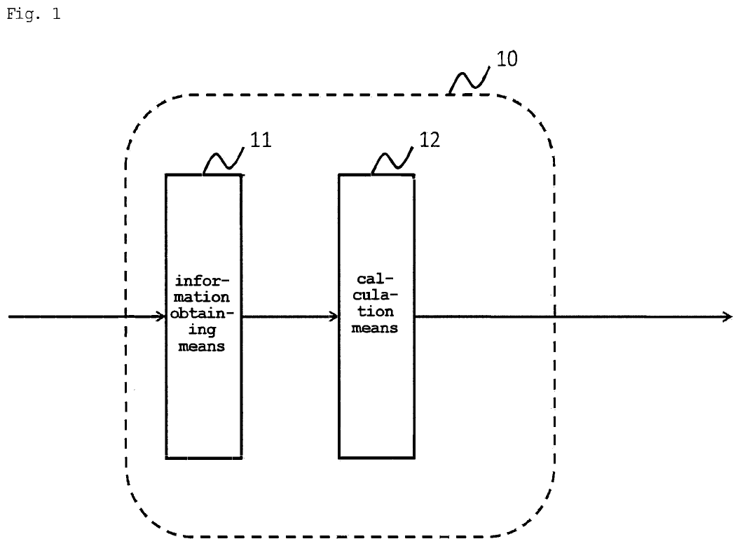

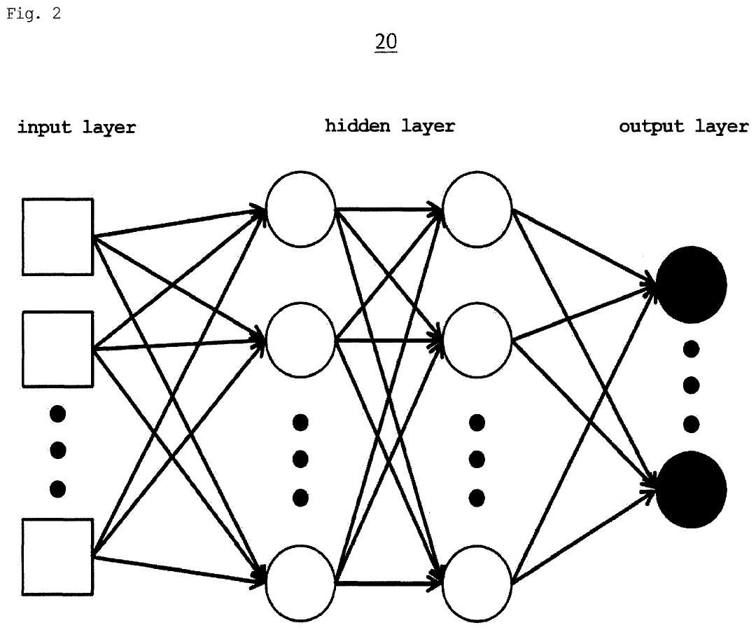

FIG. 1 shows an example of the configuration of the system 10. The system 10 includes at least information obtaining means 11 and calculation means 12.

The information obtaining means 11 is configured to acquire ocular information of a target. The information obtaining means 11 can acquire ocular information by any means. The information obtaining means 11 may acquire, for example, ocular information stored in storage means outside or inside the system 10, or acquire ocular information by extracting ocular information from an ocular information source. The ocular information source includes optical information (image or reflected light), physical information (vibration, etc.) or electrical information (potential, current, etc.), chemical information, or a combination thereof of the subject's eye. The ocular information source may be, for example, at least one image of the subject's eyeball, an electro-oculography measurement value, or motion information (e.g., acceleration measurement) of the body part of the subject, reflected light information for illumination light irradiated to the eyeball (cornea and/or sclera), current information acquired by the search coil method, or electromotive amount information obtained from a piezo element probe in direct contact to the eyeball. The reflected light information may be, for example, information obtained by a simple sensor such as a photodiode, or information obtained by image analysis. The motion information may be, for example, information obtained by image analysis or information obtained by distance measurement.

The ocular information includes, for example, information on blinking, information on pupil coordinates or relative position of the eyeball, information on eyeball movement, and information on pupil diameter. Blinks include, for example, voluntary blinks, voluntary blinks, and reflexive blinks. Examples of blink information include, but are not limited to, blink frequency or number of blinks, blink duration, time between blinks, eye closing time, eye opening speed, eye closing speed, eyelid movement width, and eyelid openness. For example, information on the pupillary coordinates or the relative position of the eyeball includes, but not limited to, the presence or absence, amount or level of movement distance, movement direction, velocity, acceleration, angular velocity, saccades, smooth eye movement, vestibulo-ocular reflex, convergence/divergence, fixational eye movement (ocular tremor, drift, microsaccades).

Information about blinking can be obtained in an eyeball image based on the pupil or eyelid in the image. For example, if the eyeball is irradiated with infrared rays, photographed, and binarized with an appropriate threshold value, the pupil is extracted as an ellipse with relatively low luminance information in the eyeball image. Ellipse fitting is performed on the binarized eyeball image, and if the ellipse fitting is performed with a certain accuracy or more (the ellipse is recognized in the image), it can be assumed that the eyes are closed, and if the accuracy is less than a certain degree (the ellipse is not recognized in the image), it can be assumed that the eyes are open. A blink can be represented as an event in which, for example, in a time window of 0.25 s, the accuracy of the ellipse fitting decreases below a certain level and then increases again above a certain level. Information related to blinking can be obtained by acquiring and analyzing the potential difference between the retina and the cornea (ocular potential) during eye movement and eyelid movement using electrodes attached to the top and bottom and/or left and right of the eye. Information about blinking can also be obtained by extracting an electro-oculography waveform characteristic of blinking. Specifically, in the electro-oculography differential waveform at the time of blinking, a sharp negative peak first appears, and then a positive peak beyond the baseline appears. This corresponds to the reciprocating motion of the eyelid, and such a positive/negative reversal of the differential waveform is not seen in typical eye movements.

In the present specification, the “blink duration” can be defined as the time required for a blink event indicated by the change in the pupil fitting level and the change in the electrooculography system as described above. The number of blinks or blink frequency can be obtained as the number of such blinking events or the number thereof divided by the window duration, for example within a three minute time window. The inter-blink time may be expressed as the time separating the blink event from the next blink event.

The eye-opening speed and eye-closing speed can be obtained by dividing the number of frames at that time, the number of frames at that time, or the eyelid movement width described later, by the time required for the accuracy of ellipse fitting to decrease below a certain level (eye-closing event) in the eyeblink event described above, and the time required for the ellipse fitting system, which decreased below a certain level, to rise above a certain level (eye opening event). The eye-opening speed and the eye-closing speed can be obtained as the time required for the electro-oculography waveform at the time of blinking to rise to the peak and the time required for the attenuation from the peak, respectively, in the case of electro-oculography.

The closed eye time is defined as the time when the eyeball area is covered by the eyelid, for example, 80% or more when the boundary between the eyelid and the eyeball is obtained by image processing such as edge detection based on luminance information in the eyeball image, and it can be extracted as a time per round or as a percentage occupied in a certain time window. In the electro-oculography, eye closing and eye opening correspond to the rise to the peak and the decay from the peak in the electro-oculography waveform, respectively, the eye-closed time can be obtained as the time span exceeding any appropriate threshold (for example, 75% of the peak of the electro-oculography waveform).

In the eyeball image, the eyelid movement width can be expressed as the actual distance corrected from the number of pixel units or the size of the captured image that the eyelid moves during a certain measurement time of interest, such as the eye-opening event or the eye-closing event described above. The width of the eyelid movement can be estimated from the peak size of the electro-oculography at the time of blinking, in electro-oculogram measurement. The eyelid opening can be expressed by the distance or the number of pixel units between two points (for example, the point where the curvature of each edge is maximized) appropriately selected from the respective edges of the upper and lower eyelids in the eyeball image.

Information about eye movement can be obtained in the eye image, for example, based on changes in pupil position information. The eyeball movement distance can be represented by the number of pixel units by which the pupil position has moved in the image in a certain time window in the case of an eyeball image, or the angular amount (amplitude) corrected in consideration of the fact that the eyeball is a sphere. Similarly, the direction of movement of the eyeball is represented as the displacement direction of the pupil position from a unit time ago when the position of the pupil in the equilateral position is set as the origin in the case of an eyeball image. In the electro-oculogram measurement, the direction and distance (amplitude) of the eye movement and the electro-oculography output are measured in advance, and by clarifying the correspondence relationship, the electro-oculography information can be converted into the eye movement information, and the movement distance and direction can be clarified.

The eyeball movement speed is expressed as a value obtained by correcting the eyeball movement distance to an arbitrary unit time, usually per second. The eyeball acceleration is expressed as a value obtained by differentiating the eyeball movement speed in arbitrary time units. The eyeball angular velocity is expressed as a value obtained by differentiating the eyeball moving direction in an arbitrary time unit. A saccade is a rapid and intermittent eye movement, reaching a maximum speed of 30 to 700 degrees/second. In the time-series data around the data point where the speed reaches a certain threshold or more, a range in which the eye movement speed continuously exceeds the stop threshold can be defined as one saccade. Smooth pursuit eye movements are smooth and gentle eye movements of the order of 30 degrees/second that capture and follow a moving object in the fovea. For example, when some visual stimulus (such as a scene seen from a train window in the real world, a point that moves smoothly on a display in an experimental environment, etc.) and the eyeball movement speed/eyeball movement direction are approximately the same, the gliding eye movement can be considered occurring. Vestibulo-ocular reflex is the movement of the eye that automatically compensates for changes in the direction of the line of sight when the head is rotated without tilting, and can be experimentally defined as an eye movement in which the movement speed/acceleration of the eye movement roughly matches the movement of the head and the movement direction is opposite. Convergence and divergence are the inward movement of both eyes when looking at near objects and the outward movement of both eyeballs when changing the fixation point from near to far, therefore, it can be detected by moving both eyeballs inward or outward in response to an approaching or receding visual stimulus, respectively. A fixational eye movement refers to minute eye movements seen under the task of fixing one point, and is classified into eye tremors, drifts, and microsaccades. A fixational eye movement may be difficult to detect from electrooculography, but it can be detected from eyeball image measurement with high temporal resolution (100 Hz or more, preferably 400 Hz or more). Ocular tremors are manifested as minute, periodic movements of 50-200 Hz and less than 0.02 degrees of travel in the frequency analysis of eye movements. Drift is a slow motion with a moving speed of about 0.015 to 0.5 degrees/second. Microsaccades move about 0.015 to 0.5 degrees, but their maximum speed reaches 20 to 100 degrees/second. A pupil diameter is obtained from the pupil image. Although the pupil image becomes an ellipse depending on the imaging direction, the pupil diameter can be estimated by correcting the ellipticity.

The information about the blink and the information about the pupil coordinates or the relative position of the eyeballs may also include calculated values of those information. The calculated value can include, for example, a function output (exponentiation, logarithm, distribution, frequency analysis value, etc.) with the information as an input variable. The calculated value can include, for example, the calculated value of the value in each division when the data is divided by a predetermined threshold (for example, the threshold for time (time window), the threshold for absolute value, the threshold for relative value) (calculated value within division). Intra-division calculated values include, but are not limited to, various statistics such as maximum value, minimum value, mean value, median value, variance, 25% percentile value, 75% percentile value, frequency analysis spectrum, and the like. The calculated values may include, for example, calculated values of intra-partition calculated values of a plurality of partitions (inter-division calculated values). Inter-interval calculated values include, but are not limited to, weighted sums, differences, time differentiations, time integrals, ratios, correlation coefficients, covariances, for example.

The ocular information may include, for example, information on blinking, information on pupillary coordinates or relative position of the eyeball, information on eyeball movement (i.e., the first parameter), or may include a calculated value (second parameter) obtained by processing the first parameter, or may include both the first parameter and the second parameter.

In a preferred embodiment, the first parameter may comprise blink duration and the second parameter may comprise the ratio of long to short blink durations. Here, the criterion for distinguishing between the long blink duration and the short blink duration may be a fixed criterion or a criterion that varies from patient to patient. The criteria that vary from patient to patient can be obtained, for example, from ocular sources obtained from the patient. The criteria that vary from patient to patient can be obtained by methods including (1) deriving time series data of blink frequency and blink duration of each blink from an ocular information source obtained from one or more patients, (2) dividing time-series data of blink frequency into a plurality of data according to the length of blink duration, (3) calculating the degree of similarity between each of the plurality of classified data and other data among the plurality of classified data adjacent in blink duration, and (4) setting thresholds for classifying blink durations based on similarity. In (2), it is preferable to divide into more data. This is because it is possible to improve the resolution for searching for the threshold value. In (3) and (4), arbitrary criteria can be used as the degree of similarity. As an example, the similarity can be a correlation coefficient. For example, if the correlation coefficient between two pieces of time-series data with different blink durations is relatively high, or if the time change patterns are similar, the two pieces of time-series data can be clustered into one cluster. On the other hand, for example, when the correlation coefficient of two time-series data with different blink durations is relatively low, or when the time change patterns are dissimilar, the two time-series data are clustered into different clusters, and a blink duration that separates the two time series data is set as a threshold that separates the blink duration. The method described above also has the advantage of being able to compress the dimensions of the data while maintaining the order of the features (here, the magnitude relationship of the duration).

The ocular information acquired by the information obtaining means 11 is passed to the calculation means 12.

The calculation means 12 is configured to calculate the estimated presence or absence, amount or level of in vivo dopamine or a substance biologically equivalent to dopamine in the subject, or a variation value thereof, based on the ocular information.

The calculation means 12 can calculate the estimated presence or absence, amount or level of in vivo dopamine or a substance biologically equivalent to dopamine in the subject, or a variation value thereof, for example, using a trained model. The trained model has trained, for example, a correlation between ocular information and the estimated presence or absence, amount or level of in vivo dopamine or a substance biologically equivalent to dopamine, or a variation value thereof.

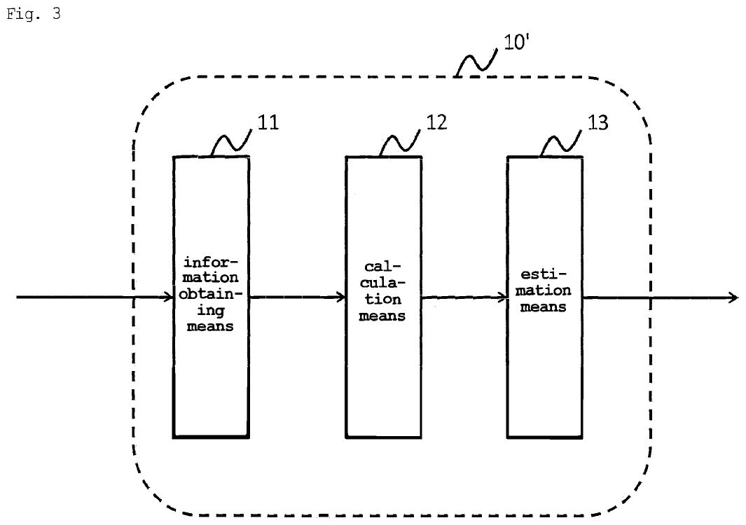

FIG. 2 shows an example of the structure of a neural network 20 for constructing a trained model that can be used by the calculation means 12.

Neural network 20 has an input layer, at least one hidden layer, and an output layer. The number of nodes in the input layer of neural network 20 corresponds to the number of dimensions of input data. The number of nodes in the output layer of neural network 20 corresponds to the number of dimensions of output data. For example, the number of nodes in the output layer is 1 when outputting the estimated presence or absence (that is, the presence or absence) of dopamine or a substance biologically equivalent to dopamine in the subject's living body. The hidden layer of neural network 20 may contain any number of nodes.

A weighting factor for each node in the hidden layer of neural network 20 may be calculated based on previously acquired data. The process of calculating this weighting factor is the training process. The training process may be supervised training or unsupervised training.

In the case of supervised training, for example, a weighting factor for each node can be calculated so that when the ocular information of a certain patient is input to the input layer, the value of the output layer is a value indicating the estimated presence/absence, amount or level of in vivo dopamine or a substance biologically equivalent to dopamine in the patient, or a variation value thereof. This can be done, for example, by backpropagation. Although it may be preferable to use a large amount of training data set for training, if the amount is too large, it is likely to fall into overfitting.

For example, a set of (teaching data for input, teaching data for output) for supervised training can be (eyeball information of the first patient, a value indicating the estimated presence or absence, amount or level of in vivo dopamine or a substance biologically equivalent to dopamine in the first patient, or a variation value thereof), (eyeball information of the second patient, a value indicating the estimated presence or absence, amount or level of in vivo dopamine or a substance biologically equivalent to dopamine in the second patient, or a variation value thereof), - - - (eyeball information of the i-th patient, a value indicating the estimated presence or absence, amount or level of in vivo dopamine or a substance biologically equivalent to dopamine in the i-th patient, or a variation value thereof), - - - or the like. When ocular information obtained from a new patient is input to the input layer of such a trained neural network model, a value showing the presence or absence, amount, or level of in vivo dopamine or a substance biologically equivalent to dopamine in the patient is output to the output layer.

The calculation means 12 may use, for example, a plurality of trained models. For example, a first trained model among the plurality of trained models can output a value indicating the estimated presence or absence of in vivo dopamine or a substance biologically equivalent to dopamine in the patient, a second trained model among the plurality of trained models can output a value indicating the amount or level of in vivo dopamine or a substance biologically equivalent to dopamine in the patient, and a third trained model among the plurality of trained models can output a value indicating the variation value of the amount or level of in vivo dopamine or a substance biologically equivalent to dopamine in the patient.

The trained models described above are not limited to neural network models as described and any other machine learning model can be utilized. For example, a random forest can be used. For example, it is possible to use a simple model constructed by simply setting weighting factors obtained by prior learning.

For example, the calculation means 12 may calculate the estimated presence or absence, amount or level of in vivo dopamine or a substance biologically equivalent to dopamine in the subject, or a variation value thereof on a rule basis without relying on machine learning.

In an example, by using a system that predicts the amount of dopamine or a substance biologically equivalent to dopamine from the amount and time course of drug administration, based on a pharmacokinetic model, or using a system that estimates the appropriate level feeling (insufficient/excessive) of dopamine or a substance biologically equivalent to dopamine only from motor symptoms, the technology of this disclosure can be implemented without using machine learning processes. Additionally, data is collected from a system implemented without using the machine learning process and the model is trained as a hypothetical correct value, alternatively, those data are used as model variables or these systems are used together for correcting the model output value, and the trained model is used, thus, the present disclosure can be performed.

The estimated presence or absence, amount or level of in vivo dopamine or a substance biologically equivalent to dopamine in the subject, or a variation value thereof, calculated by the system 10, can be used for any purpose. For example, a physician can use the values calculated by system 10 as indices for diagnosis. For example, the patient can use the value calculated by system 10 as an indicator for understanding his/her condition.

In one embodiment, the system 10′ of the present disclosure can use the estimated presence or absence, amount or level of in vivo dopamine or a substance biologically equivalent to dopamine in a subject, or a variation value thereof, to estimate patient condition. Estimating the patient's condition includes, for example, estimating the patient's dyskinesia score. In another embodiment, the system 10′ of the present disclosure can utilize the estimated presence or absence, amount or level of in vivo dopamine or a substance biologically equivalent to dopamine in the subject, or a variation value thereof, in order to estimate the estimated effective amount or effective level of a therapeutic or prophylactic agent or other medical technology for Parkinson's disease patients being treated with L-DOPA or an L-DOPA-related compound or a dopamine agonist.

FIG. 3 shows an example of the configuration of the system 10′.

The system 10′ includes at least information obtaining means 11, calculation means 12 and estimation means 13. The configuration of system 10′ is the same as that of system 10 except that estimating means 13 is provided. In FIG. 3, the same reference numerals are given to the same components as those described above with reference to FIG. 1, and the description thereof is omitted here.

The estimated presence or absence, amount or level of in vivo dopamine or a substance biologically equivalent to dopamine in the subject, or a variation value thereof, in the subject, calculated by a calculation means 12 is passed to an estimating means 13.

In one embodiment, the estimating means 13 is configured to estimate the condition of the patient based on the estimated presence or absence, amount or level of in vivo dopamine or a substance biologically equivalent to dopamine, or a variation value thereof.

The estimating means 13 can estimate the patient's condition by, for example, comparing one or more threshold values set for a patient, and the estimated presence or absence, amount or level of in vivo dopamine or a substance biologically equivalent to dopamine, or a variation value thereof. One or more threshold values set for the patient can be calculated, for example, based on ocular information acquired in advance or in real time by the information obtaining means 11 (for example, the patient's condition can be manually calibrated by a doctor or patient diary. The threshold may be set by a specialist and/or the patient himself/herself, for example, based on ocular information obtained in advance or in real time, or may be set by a trend trained from the data of the patient or other patient population in the past, by the system itself. One or more threshold values set for the patient may be calculated, for example, based on other information in addition to ocular information (e.g., the patient's condition may be manually calibrated from a physician or patient diary). Other information includes, for example, but is not limited to, information obtained by wearable devices, medication records taken by the patient, and the like. For example, when information acquired by a wearable device or information such as a medication record taken by a patient indicates a certain condition of the patient, ocular information at that time can be set as a threshold for that condition. The threshold may be, for example, a fixed value for each patient or a variable value. If it is a variable value, the threshold may vary, for example, depending on time, location, circumstances, and the like.

The estimating means 13 can also estimate the condition of a Parkinson's disease patient being treated with L-DOPA, an L-DOPA-related compound, or a dopamine agonist, for example, by the method described above.

In another embodiment, the estimating means 13 is configured to estimate an estimated effective amount or effective level of a therapeutic or prophylactic agent or other medical technology for Parkinson's disease patients being treated with L-DOPA or an L-DOPA-related compound or a dopamine agonist, based on the estimated presence or absence, amount or level of L-DOPA or an L-DOPA-related compound or a dopamine agonist in vivo, or variation values thereof.

(System for Estimating the Condition of Parkinson's Disease Patients being Treated with L-DOPA or an L-DOPA-Related Compound or a Dopamine Agonist)

The method of estimating the condition or predicting the dyskinesia score of a Parkinson's disease patient being treated with L-DOPA or an L-DOPA-related compound or a dopamine agonist as described above is performed using, for example, the system 10′ described above.

As described above, the system 10′ is configured to calculate the estimated presence or absence, amount or level of in vivo dopamine or a substance biologically equivalent to dopamine, or a variation value thereof based on the ocular information, and estimate the patient's condition based on the calculated value.

(System for Evaluating Therapeutic or Prophylactic Agent or Other Medical Technology for Parkinson's Disease Patients being Treated with L-DOPA or an L-DOPA-Related Compound or a Dopamine Agonist)

The method of evaluating therapeutic or prophylactic agent or other medical technology for Parkinson's disease patients being treated with L-DOPA or an L-DOPA-related compound or a dopamine agonist as described above is performed, for example, using the system 10′ described above.

As described above, the system 10′ is configured to calculate the estimated presence or absence, amount or level of in vivo dopamine or a substance biologically equivalent to dopamine, or a variation value thereof based on the ocular information, and to calculate the estimated effective amount or effective level of therapeutic or prophylactic agent or other medical technology based on the calculated value.

### (System Implementation Example)

The systems 10, 10′ described above may be implemented, for example, in a system 1000 including user devices and server devices connected via a network.

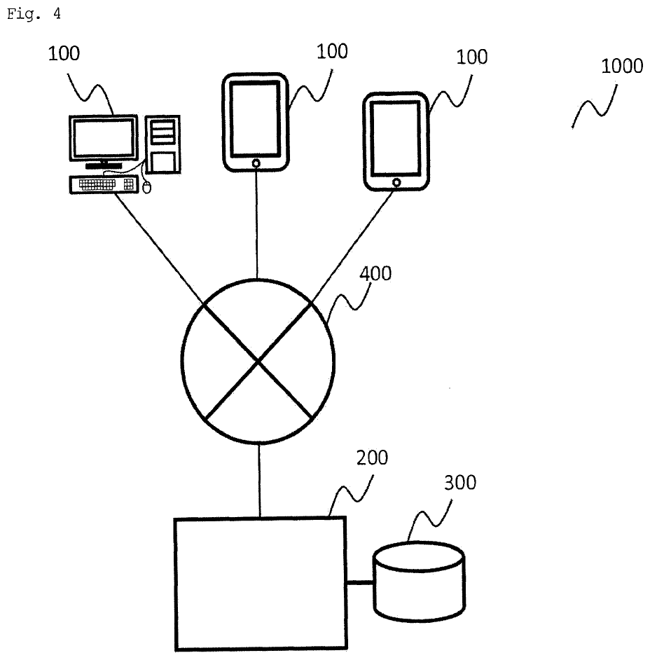

FIG. 4 is a diagram showing an example of the configuration of the system 1000 of the present disclosure.

System 1000 includes at least one user device 100, a server device 200 connected to the at least one user device 100 via network 400, and database unit 300 connected to the server device 200.

The user device 100 can be any terminal device such as smart phones, tablet computers, smart glasses, smart watches, laptop computers, desktop computers, eye trackers, and the like. The user device 100 can communicate with the server device 200 via the network 400. Here, the type of the network 400 does not matter. For example, the user device 100 may communicate with the server device 200 via internet, or may communicate with the server device 200 via a LAN. Although three user devices 100 are depicted in FIG. 4, the number of the user devices 100 is not limited to this. The number of the user devices 100 can be any number greater than or equal to one.

The server device 200 is capable of communicating with at least one user device 100 via the network 400. In addition, the server device 200 can communicate with the database unit 300 connected to the server device 200.

For example, the database unit 300 connected to the server device 200 may store ocular information of a plurality of targets acquired in advance. A plurality of stored ocular information can be utilized, for example, to build a trained model. For example, the database unit 300 may store the built trained model.

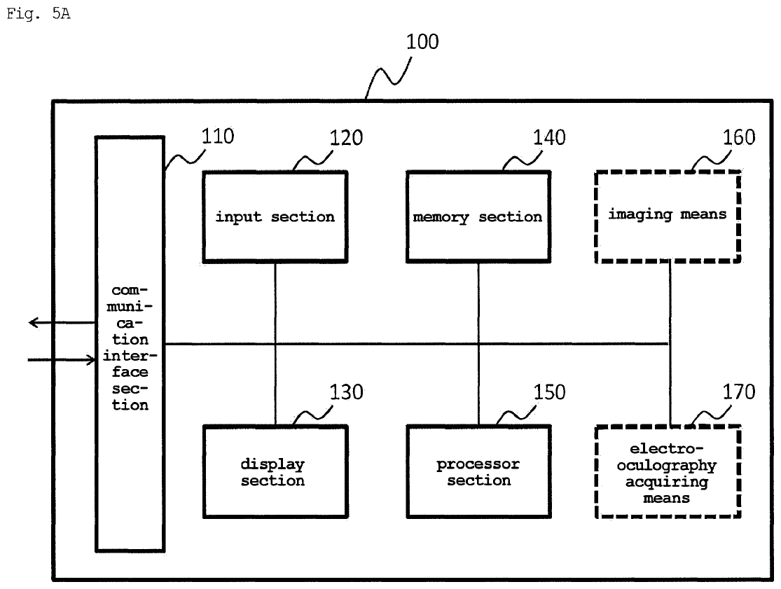

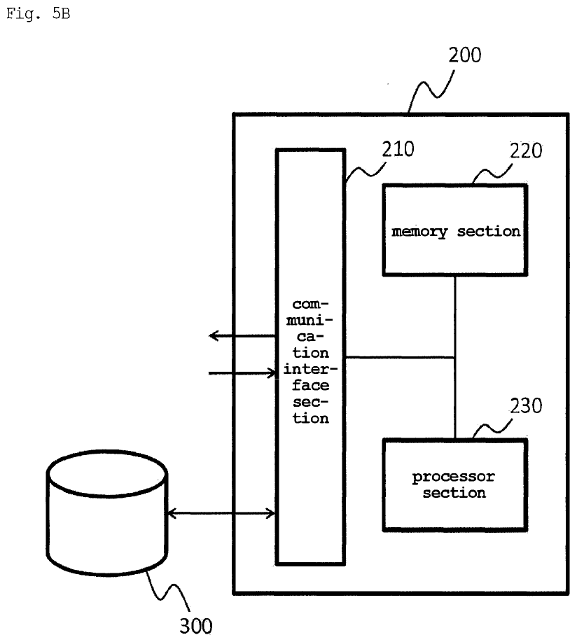

FIG. 5A shows an example of the configuration of the user device 100.

The user device 100 has a communication interface section 110, an input section 120, a display section 130, a memory section 140 and a processor section 150.

The communication interface unit 110 controls communication via the network 400. The processor unit 150 of the user device 100 can receive information from outside the user device 100 and can transmit information to the outside of the user device 100 via the communication interface unit 110. For example, the processor unit 150 of the user device 100 can receive information from the server device 200 and can transmit information to the server device 200 via the communication interface unit 110. The communication interface section 110 may control communications in any manner.

The input unit 120 allows a user to input information into the user device 100. It does not matter in what manner the input unit 120 enables the user to input information to the user device 100. For example, if the input unit 120 is a touch panel, the user may input information by touching the touch panel. Alternatively, if the input unit 120 is a mouse, the user may input information by operating the mouse. Alternatively, if the input unit 120 is a keyboard, the user may input information by pressing keys on the keyboard. Alternatively, if the input unit 120 is a microphone, the user may input information by voice.

The display 130 may be any display for displaying information.

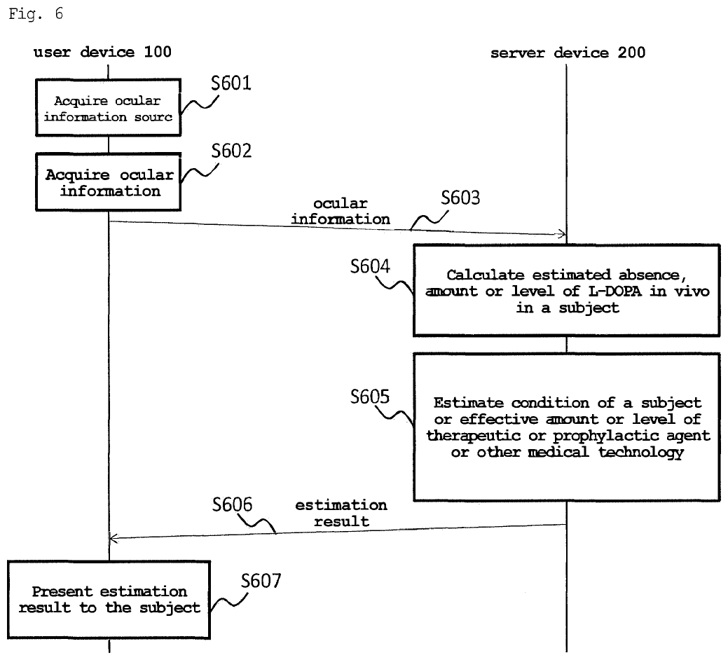

The memory unit 140 stores programs for executing processes in the user device 100, data required for executing the programs, and the like. The memory unit 140 stores, for example, a part or all of a program for predicting the presence or absence, amount or level of in vivo dopamine or a substance biologically equivalent to dopamine in the subject, or a variation thereof, and a program for estimating the patient's condition, and/or a program for evaluating therapeutic or prophylactic agent or other medical technology for Parkinson's disease patients being treated with L-DOPA or an L-DOPA-related compound or a dopamine agonist (e.g., program for executing the processing shown in FIGS. 6 to 8 described later). The memory unit 140 may store applications that implement arbitrary functions. The memory unit 140 may store, for example, an application for acquiring ocular information by tracking a pupil in an eyeball image (eye tracking). As a result, the user device 100 has a portion that implements the eye tracking function. The memory unit 140 may store, for example, an application for acquiring ocular information from electro-oculography measurement values. Thereby, the user device 100 has a part that implements the function of measuring the electro-oculography. In addition, the memory unit 140 may store an application for obtaining ocular information by other means. Here, it does not matter how the program is stored in the memory unit 140. For example, the program may be pre-installed in memory unit 140. Alternatively, the program may be installed in memory unit 140 by being downloaded via network 400. Memory unit 140 may be implemented by any storage means.

The processor unit 150 controls the operation of the user device 100 as a whole. The processor unit 150 reads a program stored in the memory unit 140 and executes the program. This allows the user device 100 to function as a device that executes desired steps. The processor unit 150 may be implemented by a single processor or multiple processors.

The user device 100 can include imaging means 160 in addition to the configuration described above. Imaging means 160 is any means for acquiring an image. The imaging means 160 is, for example, a camera. Here, “image” includes still images and moving images. The imaging unit 160 may be, for example, a camera built into the user device 100 or an external camera attached to the user device 100. For example, when the user device 100 is a smart phone, a camera built into the smart phone can be used as the imaging means 160.

The user device 100 can include an electro-oculogram acquiring means 170 in addition to the above-described configuration or instead of the imaging means 160 described above. The electro-oculography acquiring means 170 is any means for acquiring electro-oculography. The electro-oculogram acquiring means 170 is, for example, a plurality of electrodes that can be placed on the skin around the eyes. For example, the electro-oculogram acquiring means 170 may be an electrode built into the user device 100 or an external electrode attached to the user device 100. For example, when the user device 100 is a spectacles-type eye tracker, electrodes built into the spectacles-type eye tracker can be used as the electro-oculogram acquiring means 170.

Although each component of the user device 100 is provided in the user device 100 in the example shown in FIG. 5A, the present disclosure is not limited to this. Any of the components of user device 100 may be provided external to user device 100. For example, when the input unit 120, the display unit 130, the memory unit 140, the processor unit 150, and the imaging unit 160 are each configured by separate hardware components, each hardware component may be connected via an arbitrary network. At this time, the type of network does not matter. Each hardware component may be connected via a LAN, wirelessly, or wired, for example. The user device 100 is not limited to a particular hardware configuration. For example, it is within the scope of the present disclosure to configure the processor unit 150 with analog circuitry rather than digital circuitry. The configuration of the user device 100 is not limited to those described above as long as its functions can be realized.

FIG. 5B shows an example of the configuration of the server device 200.

The server device 200 includes a communication interface section 210, a memory section 220 and a processor section 230.

The communication interface section 210 controls communication via network 400. The communication interface section 210 also controls communication with the database section 300. The processor unit 230 of the server device 200 can receive information from outside the server device 200 via the communication interface unit 210 and can transmit information to the outside of the server device 200. The processor unit 230 of the server device 200 can receive information from the user device 100 via the communication interface unit 210 and can transmit information to the user device 100. The communication interface unit 210 may control communications in any manner.

The memory unit 220 stores programs required for executing processes of the server device 200, data required for executing the programs, and the like. For example, a part or all of a program for predicting the presence or absence, amount or level of in vivo dopamine or a substance biologically equivalent to dopamine in a subject, or a variation thereof, a program for estimating the patient's condition, and/or a program for evaluating therapeutic or prophylactic agent or other medical technology for Parkinson's disease patients being treated with DOPA or L-DOPA-related compounds or dopamine agonists (e.g., program for executing the processing shown in FIGS. 6 to 8 below), are stored. The memory unit 220 may store, for example, an application for acquiring ocular information by tracking pupils in an eyeball image (eye tracking), and an application for acquiring ocular information from electro-oculography measurement values. The memory unit 220 may store, for example, an application for acquiring ocular information by other means. The memory unit 220 may be implemented by any storage means.

The processor unit 230 controls the operation of the server device 200 as a whole. The processor unit 230 reads a program stored in the memory unit 220 and executes the program. This allows the server device 200 to function as a device that executes desired steps. The processor unit 230 may be implemented by a single processor or multiple processors.

In the example shown in FIG. 4B, each component of the server device 200 is provided within the server device 200, but the present disclosure is not limited to this. Any one of the components of server device 200 may be provided outside server device 200. For example, when the memory unit 220 and the processor unit 230 are each composed of separate hardware components, each hardware component may be connected via an arbitrary network. At this time, the type of network does not matter. Each hardware component may be connected via a LAN, wirelessly, or wired, for example. The server device 200 is not limited to a specific hardware configuration. For example, it is within the scope of the present disclosure to configure the processor portion 230 with analog circuitry rather than digital circuitry. The configuration of the server device 200 is not limited to the above as long as the functions can be realized.

In the examples illustrated in FIGS. 4 and 5B, the database unit 300 is provided outside the server device 200, but the present disclosure is not limited to this. It is also possible to provide the database unit 300 inside the server device 200. At this time, the database section 300 may be implemented by the same storage means as the storage means in which the memory section 220 is implemented, or by a storage means different from the storage means in which the memory section 220 is implemented. In any case, the database unit 300 is configured as a storage unit for the server device 200. The configuration of the database unit 300 is not limited to a specific hardware configuration. For example, the database unit 300 may be configured with a single hardware component, or may be configured with a plurality of hardware components. For example, the database unit 300 may be configured as an external hard disk device of the server device 200, or configured as a cloud storage connected via a network.

The components of the system 10 may be provided by the user device 100, may be provided by the server device 200, or may be distributed to both the user device 100 and the server device 200, for example. When the components of the system 10 are distributed to both the user device 100 and the server device 200, the user device 100 can be provided with the information obtaining means 11 and the server device 200 can be provided with the calculation means 12.

Similarly, the components of the system 10′ may be provided by the user device 100, may be provided by the server device 200, or may be distributed to both the user device 100 and the server device 200. When the components of the system 10′ are distributed to both the user device 100 and the server device 200, the user device 100 may be provided with the information obtaining means 11, and the server device 200 may be provided with the calculation means 12 and the estimation means 13, alternatively, the information obtaining means 11 and the calculation means 12 may be provided in the user device 100, and the estimation means 13 may be provided in the server device 200.

In one embodiment, in the system 10′, the user device 100 is provided with the information obtaining means 11, and the server device 200 is provided with the calculation means 12 and the estimation means 13.

FIG. 6 is a data flow diagram showing data exchange between the user device 100 and the server device 200 in this embodiment.

In step S601, the user device 100 acquires an ocular information source. The ocular information source may be, for example, at least one image of the subject's eyeball, or may be an electro-oculogram measurement value. Preferably, the ocular information source may be a plurality of images taken of the subject's eye or electro-oculogram measurements in time series. This is because multiple images or time-series electro-oculography measurements can include a time component of ocular information.

For example, the imaging unit 160 of the user device 100 can acquire at least one image by photographing an eyeball. For example, the image capturing unit 160 of the user device 100 may capture the eyeball of the target when the target is recognizing such as when the target is staring at the image capturing unit 160, or may photograph the eyeballs of the object when the object is not recognizing, such as when the object is within the field of view of the imaging means 160. By photographing the eyeballs of the target when the target is not recognizing it, the ocular information source can be obtained without imposing a burden on the target.

For example, the electro-oculogram acquisition unit 170 of the user device 100 can acquire an electro-oculography measurement value by measuring the electrical potential around the eye. For example, the electro-oculogram acquisition unit 170 of the user device 100 may acquire the electro-oculography measurement value when the subject is recognizing, such as when the subject attaches electrodes around the eye, or may acquire the electro-oculography measurement value using electrodes or the like built into the spectacle-type device worn by the subject when the subject is not recognizing. By photographing the eyeballs of the target when the target is not recognizing it, the ocular information source can be obtained without imposing a burden on the target.

In step S602, the user device 100 acquires ocular information. For example, the information acquisition unit 11 that the processor unit 150 of the user device 100 may include acquires ocular information from the ocular information source acquired in step S601. Eyeball information includes, for example, information on blinking, information on pupil coordinates or relative positions of eyeballs, and information on eyeball movements. The information obtaining means 11 can acquire ocular information from an ocular information source by any known method, for example.

In step S603, the user device 100 transmits ocular information to the server device 200. For example, the processor unit 150 of the user device 100 transmits ocular information to the server device 200 via the communication interface unit 110. The server device 200 receives the transmitted ocular information. For example, the processor unit 230 of the server device 200 receives ocular information from the user device 100 via the communication interface unit 210.

In step S604, the server device 200 calculates the estimated presence or absence, amount, or level of in vivo dopamine or a substance biologically equivalent to dopamine in the subject based on the ocular information. For example, the calculation means 12 that can be included in the processor unit 230 of the server device 200 estimates the presence or absence, amount or level of in vivo dopamine or a substance biologically equivalent to dopamine in the subject based on the ocular information received in step the S603.

The calculation means 12 can, for example, calculate the estimated presence or absence, amount, or level of in vivo dopamine or a substance biologically equivalent to dopamine in the subject, using a trained model. The trained model has trained a correlation between the ocular information and the estimated presence or absence, amount, or level of in vivo dopamine or a substance biologically equivalent to dopamine in the subject, and when the ocular information received in step S603 is input into the trained model, the estimated presence or absence, amount, or level of in vivo dopamine or a substance biologically equivalent to dopamine in the corresponding organism is output.

In step the S605, the server device 200 estimates the condition of the subject based on the estimated presence or absence, amount, or level of in vivo dopamine or a substance biologically equivalent to dopamine in the subject.

Alternatively, if the subject is a Parkinson's disease patient who is being treated with L-DOPA, an L-DOPA-related compound, or a dopamine agonist, the server device 200 estimates the estimated effective amount or effective level of a therapeutic or prophylactic agent or other medical technology to the patient. For example, the estimating means 13 that can be included in the processor unit 230 of the server device 200 estimates the patient's condition or estimates the estimated effective amount or effective level of a therapeutic or prophylactic agent or other medical technology for a patient, based on the estimated presence or absence, amount, or level of in vivo dopamine or a substance biologically equivalent to dopamine in the subject calculated in the step S604.

The estimating means 13 can, for example, estimate the patient's condition by comparing one or more threshold values set for a patient, and the estimated presence or absence, amount or level of in vivo dopamine or a substance biologically equivalent to dopamine, or a variation value thereof. One or more threshold values set for a patient can be calculated based on ocular information acquired in advance or in real time by the information acquiring means 11, for example. The threshold may be set by the specialist and/or the patient himself, for example, based on ocular information obtained in advance or in real time, or set by the system itself based on the tendency trained in the past. One or more thresholds set for the patient may be calculated based on other information in addition to the ocular information, for example. Other information includes, for example, but is not limited to, information obtained by wearable devices, medication records taken by the patient, and the like. For example, when information acquired by a wearable device or information such as a medication record taken by a patient indicates a certain condition of the patient, the ocular information at that time can be set as a threshold for that condition. The threshold may be, for example, a fixed value for each patient or a variable value. If it is a variable value, the threshold may vary, for example, depending on time, location, circumstances, and the like.

The estimating means 13 can also estimate the condition of a Parkinson's disease patient being treated with L-DOPA, an L-DOPA-related compound, or a dopamine agonist, for example, by the method described above.

In another embodiment, the estimating means 13 is configured to estimate an estimated effective amount or effective level of a therapeutic or prophylactic agent or other medical technology for a Parkinson's disease patient being treated with L-DOPA or an L-DOPA-related compound or a dopamine agonist, based on the estimated presence or absence, amount or level of in vivo dopamine or a substance biologically equivalent to dopamine, or a variation value thereof.

In step S606, the server device 200 transmits the estimated result in the step S605 to the user device 100. For example, the processor unit 230 of the server device 200 transmits the estimated result in the step S605 to the user device 100 via a communication interface unit 210. The user equipment 100 receives the transmitted estimated result. For example, the processor unit 150 of the user device 100 receives the estimated result from the server device 200 via the communication interface unit 110.

In step S607, the user device 100 presents the estimated result received in the step S606 to the target. The mode of presentation does not matter. For example, the display unit 130 of the user device 100 can display the estimated result. Alternatively, a microphone, which may be included in or connected to the user device 100, may present the estimated results audibly. Alternatively, a printer, which may be included in or connected to user device 100, may print the estimated result.

This allows a subject (or a doctor, nurse, or other health care professional who sees the subject) to know the subject's condition, or an estimated effective amount or effective level of a therapeutic or prophylactic agent or other medical technology for the subject.

In this embodiment, the user device 100 can obtain the estimated result estimated from the ocular information only by acquiring the ocular information by the user device 100 and transmitting it to the server device 200. The user device 100 does not need to bear the calculation load on the calculation means 12 and the estimating means 13 either.

In another embodiment, in the system 10′, the user device 100 can be provided with the information obtaining means 11 and the calculation means 12, and the server device 200 can be provided with the estimation means 13.

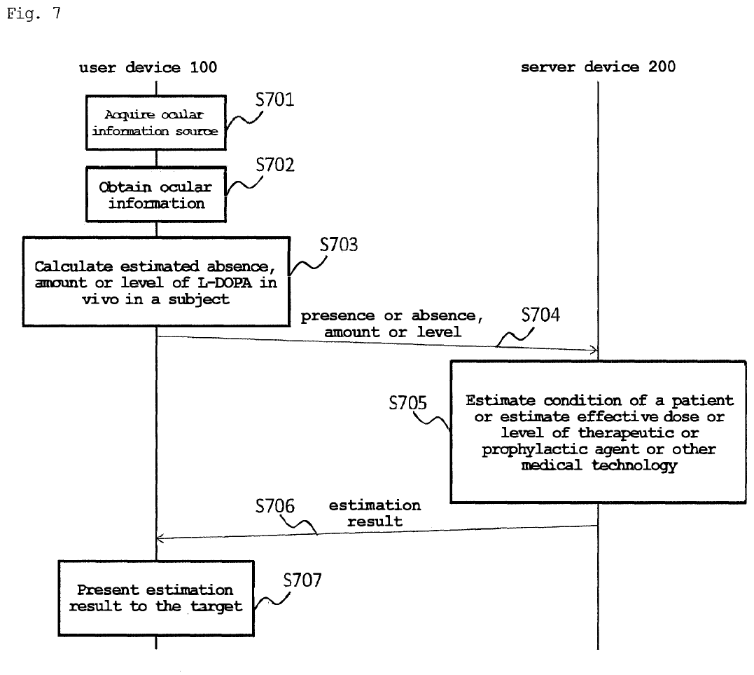

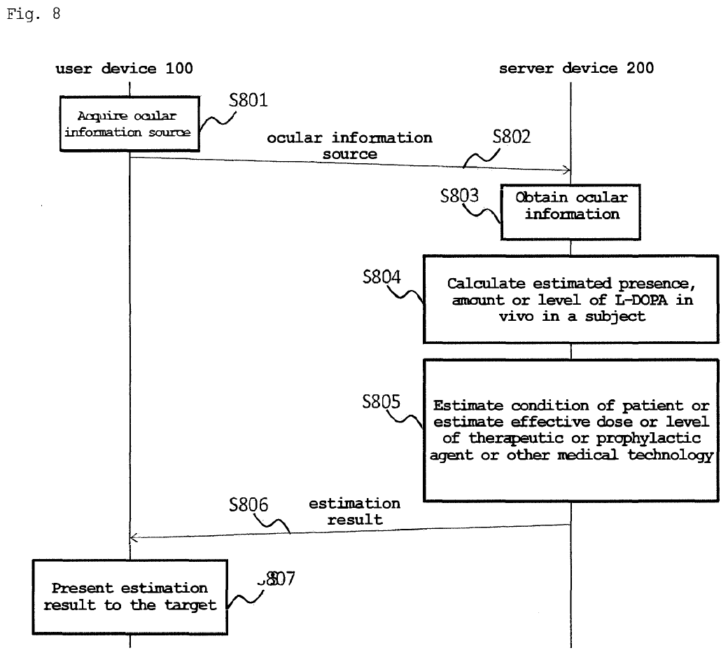

FIG. 7 is a data flow diagram showing exchange of data between the user device 100 and the server device 200 in this embodiment.

In step S701, the user device 100 acquires an ocular information source. The step S701 is a step similar to the step S601.

In step S702, the user device 100 acquires ocular information. The step S702 is a step similar to the step S602.

In step S703, the user device 100 calculates the estimated presence or absence, amount, or level of in vivo dopamine or a substance biologically equivalent to dopamine in the subject based on the ocular information. For example, the calculation means 12 that can be included in the processor unit 150 of the user device 100 estimates the estimated presence or absence, amount or level of in vivo dopamine or a substance biologically equivalent to dopamine in the target based on the ocular information received in the step S603.

The calculation means 12 can, for example, calculate the estimated presence or absence, amount, or level of in vivo dopamine or a substance biologically equivalent to dopamine in the subject, using a trained model. The trained model has trained a correlation between the ocular information and the estimated presence or absence, amount, or level of in vivo dopamine or a substance biologically equivalent to dopamine in the subject, and when the ocular information acquired in the step S702 is input into the trained model, the estimated presence or absence, amount, or level of in vivo dopamine or a substance biologically equivalent to dopamine in the corresponding organism is output.

In step S704, the user device 100 transmits to the server device 200 information indicating the estimated presence or absence, amount, or level of in vivo dopamine or a substance biologically equivalent to dopamine calculated in the step S703. For example, the processor unit 150 of the user device 100 transmits information indicating the calculated presence or absence, amount, or level of in vivo dopamine or a substance biologically equivalent to dopamine to the server device 200, via the communication interface unit 110. The server device 200 receives information indicating the estimated presence or absence, amount, or level of in vivo dopamine or a substance biologically equivalent to dopamine transmitted. For example, the processor unit 230 of the server device 200 receives from the user device 100 information indicating the estimated presence or absence, amount, or level of in vivo dopamine or a substance biologically equivalent to dopamine via the communication interface unit 210.

In step S705, the server device 200 estimates the condition of the subject based on the estimated presence or absence, amount, or level of in vivo dopamine or a substance biologically equivalent to dopamine in the subject. Alternatively, if the subject is a Parkinson's disease patient who is being treated with L-DOPA, an L-DOPA-related compound, or a dopamine agonist, the server device 200 estimates an estimated effective amount or effective level of a therapeutic or prophylactic agent or other medical technology to the patient. For example, the estimating means 13, which may be included in the processor unit 230 of the server device 200, estimates the patient's condition based on the estimated presence or absence, amount, or level of in vivo dopamine or a substance biologically equivalent to dopamine in the subject received in the step S704, or estimates an estimated effective amount or effective level of a therapeutic or prophylactic agent or other medical technology for the patient. The step S705 is a step similar to the step S605.

At step S706, the server device 200 transmits the estimated result in the step S605 to the user device 100. The step S706 is a step similar to the step S606.

In step S707, the user device 100 presents the estimated result received in the step S606 to the target. The step S707 is a step similar to the step S607.

This allows a subject (or a doctor, nurse, or other health care professional who sees the subject) to know the subject's condition, or an estimated effective amount or effective level of a therapeutic or prophylactic agent or other medical technology for the subject.

In this embodiment, the user device 100 acquires ocular information, calculates the estimated presence or absence, amount or level of in vivo dopamine or a substance biologically equivalent to dopamine, and transmits this to the server device 200, thereby the user device 100 can obtain an estimated result estimated from the estimated presence or absence, amount, or level of in vivo dopamine or a substance biologically equivalent to dopamine.

In another embodiment, the server device 200 can comprise the information obtaining means 11, the calculation means 12, and the estimation means 13 of the system 10′.

FIG. 8 is a data flow diagram showing exchange of data between the user device 100 and the server device 200 in this embodiment.

In step S801, the user device 100 acquires an ocular information source. The step S801 is a step similar to the step S601.

In step S802, the user device 100 transmits the ocular information source acquired in the step S801 to the server device 200. For example, the processor unit 150 of the user device 100 transmits the ocular information source to the server device 200 via the communication interface unit 110. The server device 200 receives the transmitted ocular information source. For example, the processor unit 230 of the server device 200 receives the transmitted ocular information source from the user device 100 via the communication interface unit 210.

In step S803, the server apparatus 200 acquires ocular information. For example, the information acquisition unit 11 that can be included in the processor unit 230 of the server device 200 acquires the ocular information from the ocular information source received in the step S802. The ocular information includes, for example, information on blinking, information on pupil coordinates or relative positions of eyeballs, and information on eyeball movements. The information obtaining means 11 can acquire ocular information from an ocular information source by any known method, for example.

In step S804, the server device 200 calculates the estimated presence or absence, amount, or level of in vivo dopamine or a substance biologically equivalent to dopamine in the subject based on the ocular information. The step S804 is a step similar to the step S604.

In step S805, the server device 200 estimates the condition of the subject based on the estimated presence or absence, amount, or level of in vivo dopamine or a substance biologically equivalent to dopamine in the subject. Alternatively, if the subject is a Parkinson's disease patient who is being treated with L-DOPA, an L-DOPA-related compound, or a dopamine agonist, the server device 200 estimates an estimated effective amount or effective level of a therapeutic or prophylactic agent or other medical technology to the patient. For example, the estimating means 13 that can be included in the processor unit 230 of the server device 200 estimates the patient's condition based on the estimated presence or absence, amount, or level of in vivo dopamine or a substance biologically equivalent to dopamine in the subject calculated in the step S804, or estimates an estimated effective amount or effective level of a therapeutic or prophylactic agent or other medical technology for the patient. The step S805 is a step similar to the step S805.

In step S806, the server device 200 transmits the estimated result in the step S805 to the user device 100. The step S806 is a step similar to the step S606.

In step S807, the user device 100 presents the estimated result received in the step S806 to the target. The step S807 is a step similar to the step S607.

This allows a subject (or a doctor, nurse, or other health care professional who sees the subject) to know the subject's condition, or an estimated effective amount or effective level of a therapeutic or prophylactic agent or other medical technology for the subject.

In this embodiment, the user device 100 can obtain the estimated result estimated from the ocular information source only by acquiring the ocular information source by the user device 100 and transmitting it to the server device 200. The user device 100 does not need to bear the calculation load for acquiring the ocular information in addition to the calculation load on the calculation means 12 and the estimation means 13.

In another embodiment, the user device 100 may comprise the information obtaining means 11, the calculation means 12 and the estimation means 13 of the system 10′. At this time, the user device 100 does not need to communicate with the server device 200 and can operate standalone. The user device 100, for example, performs the processes of the steps S601 to S607 other than the steps S603 and S606.

Although the examples described above with reference to FIGS. 6 to 8 describe the operations occurring in a particular order, the order of processes is not limited to that described and can be performed in any order that is logically possible.

In the examples described above with reference to FIGS. 6 to 8, at least one of the processes of each step shown in FIGS. 6 to 8 can be executed by a program stored in the processor unit 150 and the memory unit 140 of the used device 100, and/or the processor unit 230 and the memory unit 220 of the server device 200, but the present disclosure is not limited thereto. At least one of the processes of each step shown in FIGS. 6 to 8 may be implemented by a hardware configuration such as a control circuit.

The system of the present disclosure can be used in combination with, for example, a means capable of outputting indices related to Parkinson's disease.

In one example, the means capable of outputting Parkinson's disease indices is a wearable device configured to be worn on the patient's body.

The wearable device may be attached to at least one limb of the patient and configured to measure limb movement (e.g., tremor) due to Parkinson's disease. The wearable device can measure limb movements and output velocity data, acceleration data, angular velocity data, and/or angular acceleration data, for example, by internally mounted inertial sensors. Such wearable devices whose output data are analyzed and used as indices for Parkinson's disease may be devices described, for example, in Rob Powers1 et al., “Smartwatch inertial sensors continuously monitor real-world motor variations in Parkinson's disease”, Sci. Transl. Med. 13, eabd7865 (2021) 3 Feb. 2021.

By using such a wearable device to obtain an indicator related to the patient's Parkinson's disease in advance, the indicator can be used to calculate a threshold for estimating the patient's condition.

For example, the wearable device and the information obtaining means 11 are used together to continuously acquire motion measurement data and ocular information. The ocular information when the movement measurement data indicates that Parkinson's disease is present can be set as a threshold for Parkinson's disease symptoms.

The systems, apparatus, devices of the present disclosure may optionally be configured with one or more sensors that collect user health data other than indices related to Parkinson's disease. Health data may include any suitable data related to the user's health. In some examples, the device may be configured to capture health data from the user. Such health data may include pulse rate, heart rate, heart rate variability measurements, temperature data, number of steps, standing and sitting times, calories burned, minutes exercised, and/or any other suitable health data for the user. The device may be configured with one or more input devices that allow a user to interact with the device. The device may also be configured with one or more output devices for outputting any suitable output information. For example, the device may be configured to output visual information, audio information, and/or tactile information. In some examples, the output information can be presented to the user in a manner that induces the user to perform one or more actions related to breathing. For example, the output information may include a varying progress indicator (e.g., some kind of visual information). The progress indicator can be presented on the device's graphical user interface and can be configured to guide the user through a series of respiratory movements included in the respiratory sequence, as further described herein. The output information may be presented by an application running on the device.

The device may be associated with a second device (e.g., a paired device (e.g., a wearable device such as a watch-type device or eyeglass-type device) or a host device). In some examples, this may include the device being paired with the second device in any suitable manner. Pairing two devices optionally allows a second device to act as a replacement for this device. The device, the second device, or any suitable combination of the device and the second device can generate output information based at least in part on the health data. Arbitrary electrodes may be placed to obtain further parameters. Health indices that can be calculated using electrodes include, but are not limited to, cardiac function (ECG, EKG), water content, body fat percentage, galvanic skin resistance, and combinations thereof.

The present disclosure may apply systems, apparatus, and methods for the operation of wearable devices that depend on whether the wearable device that the present disclosure utilizes is worn. Such wearable devices can be attached to a portion of the user's body by an attachment member and can operate in at least a connected condition and a disconnected condition. One or more sensors disposed within the wearable device and/or mounting member can detect when the device is attached to or in close proximity to a subject. One or more sensors disposed within the wearable device and/or attachment member can detect that the attached object, if present, is part of the user's body. Such a technique is publicly known, and for example, the technique described in WO2015/116163 can be used.

It may be linked or used together with an electronic medical record application such as Welby. For example, at home, it is possible to periodically take pictures of eye movements for about 5 minutes with a smartphone camera function or a stationary eye tracker, and record parameters, and by combining with the drug intake record and patient diary (symptom) data originally installed in the app, it optimizes a correlation between symptoms and eye movement parameters. Data can be transmitted remotely to allow physicians to optimize drug prescribing. A contactless eye tracker or an eye tracker available from Tobii Technology (Sweden) can be used here.

In one embodiment, the present disclosure provides a system for implementation as an application, and this system is a system used for the treatment of treatable diseases, and the system comprises a server and a user terminal, and medical characteristics indicative of patient medical characteristics associated with a disease are clustered into various medical characteristics (e.g., behavioral medical characteristics, knowledge medical characteristics, and cognitive medical characteristics); the server stores a plurality of medical characteristics, each associated with one or more treatments, each medical trait is further associated with another type of medical trait (e.g., any one of behavioral, knowledge, and cognitive medicine traits); the server further selects a therapeutic method associated with a particular medical characteristic selected from the plurality of medical characteristics for treatment and a therapeutic method for performing from a therapy associated with each of the various medical characteristic information associated with the selected medical characteristic, and transmits therapy information for the selected therapy; and the user terminal can be employed to present information for therapy based on the therapy information received. In one embodiment, the selection of a treatment method by a medical professional serves as teacher information, and changes a treatment selection probability, therefore, the probability of selecting a treatment that is considered to be highly effective increases, and it becomes possible to perform more effective treatment. Alternatively, clustering factors for individual patients can be modified based on effect information. For example, the cluster factor of the cluster to which the medical property associated with the therapy that was found to be effective belongs is increased, and decreased if there is no effect. Treatment for which cluster is effective may vary from patient to patient. Since the cluster to which the medical property associated with the effective treatment belong is highly likely to be an effective cluster for the patient, by increasing the cluster factor, the treatment method selection probability for the cluster increases and a more effective treatment is made possible.

### NOTE

As used herein, “or” is used when “at least one or more” of the listed matters in the sentence can be employed. When explicitly described herein as “within the range of two values”, the range also includes the two values themselves.

Reference literatures such as scientific literatures, patents, and patent applications cited herein are incorporated herein by reference to the same extent that the entirety of each document is specifically described.

As described above, the present disclosure has been described while showing preferred embodiments to facilitate understanding. The present disclosure described hereinafter is based on the Examples. The above descriptions and the following Examples are not provided to limit the present disclosure, but for the sole purpose of exemplification. The present invention will be described in more detail below by means of Reference Examples, Examples and Test Examples, which are provided for illustrative purposes only and are not intended to limit the present disclosure. The compound names shown in the following Reference Examples and Examples do not necessarily follow the IUPAC nomenclature. Abbreviations may be used for simplification of the description, but these abbreviations have the same meanings as those described above. The scope of the present disclosure is not limited to the embodiments and Examples specifically described herein and is limited only by the scope of claims.

### Example

The Examples are described hereinafter. The animals used in the following Examples were handled in compliance with Japanese and other laws and regulations and the spirit of animal welfare, and in the case of humans, in compliance with the Declaration of Helsinki. While the specific products described in the Examples were used, reagents can be substituted with an equivalent product from another manufacturer (Sigma-Aldrich, Wako Pure Chemical, Nacalai Tesque, R & D Systems, or the like).

### Reference Example

### Reference Example 1: Evaluation of Striatal Dopamine Releasing Action in PD-LID Model Rats

### (Test Method)

As a representative experimental model for Parkinson's disease, a rat striatal dopaminergic neuron destruction model is known in which 6-hydroxydopamine (hereinafter sometimes referred to as “6-OHDA”) is locally administered to one side of the brain (6-OHDA unilateral treated rat (6-OHDA injured rat)). Male Wistar rats (12 weeks old, Japan SLC, Inc.) were used to prepare model animals. Desipramine hydrochloride (25 mg/kg; Wako Pure Chemical Industries, Ltd.) was intraperitoneally administered, and 30 minutes after the administration, inhalation anesthesia with isoflurane was performed using a general anesthesia machine for experimental animals. Under isoflurane anesthesia, the rat was fixed in a stereotaxic apparatus, the skin of the head was incised with a surgical scalpel to expose the cranial bone, and the coordinate of bregma, which is the starting point (AP: 0, ML: 0, DV: 0), was confirmed, and the coordinate of the right medial forebrain bundle (AP: −4.4 mm, ML: 1.5 mm, DV: 7.8 mm from bregma) was measured. After inserting the injection tube for administration into the measuring coordinates, 6-OHDA (9 μg/4 μL; Sigma-Aldrich) having a dopaminergic neurodegenerative effect was locally injected. Two weeks after the surgery, apomorphine hydrochloride 0.5 hydrate (0.5 mg/kg; Wako Pure Chemical Industries) was administered subcutaneously, rotational movement to the contralateral side with respect to the site injected with 6-OHDA was observed, and rats that rotated 7 times or more per minute were used as 6-OHDA unilateral treated rats. In order to prepare a PD-LID model rat, a mixed solution of levodopa methyl ester hydrochloride (6 mg/kg; Sigma-Aldrich) and benserazide hydrochloride (15 mg/kg; Sigma-Aldrich) dissolved in physiological saline (hereinafter sometimes referred to as a “levodopa blended liquid”) was intraperitoneally administered to 6-OHDA-injured rats once a day. Repeated administration of the levodopa blended liquid was performed for 3 weeks or more.

In order to measure striatal dopamine release, guide cannula implantation into the striatum of PD-LID model rats and chromatography were performed by a method described in a reference (Pharmacol Res Perspect. 2015 June; 3(3): e00142.). On the day of testing, a dialysis probe was inserted into the striatum along the guide cannula. The tape was applied to the abdomen of the rat at 60 cm²/kg (containing 6.5% W/V tandospirone free form). Four hours after application, the levodopa solution was intraperitoneally administered, and the dialysate was collected in sample vials every 10 minutes. Using an HPLC-ECD system (EiCOM Co., Ltd.), the amount of dopamine in the collected dialysate was measured. The results in the figure are shown as mean±standard error. Individuals from whom 50% or more of the tape was removed during the test were excluded from the analysis. Statistical analysis of study results was performed by comparing with the placebo tape administration group by t-test. * indicates p<0.05, meaning that there is a significant difference from the placebo tape administration group.

### (Result)

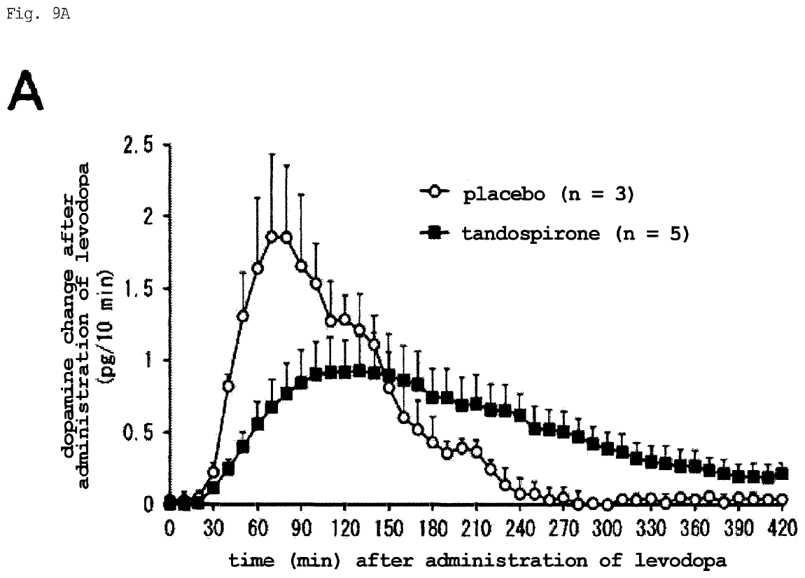

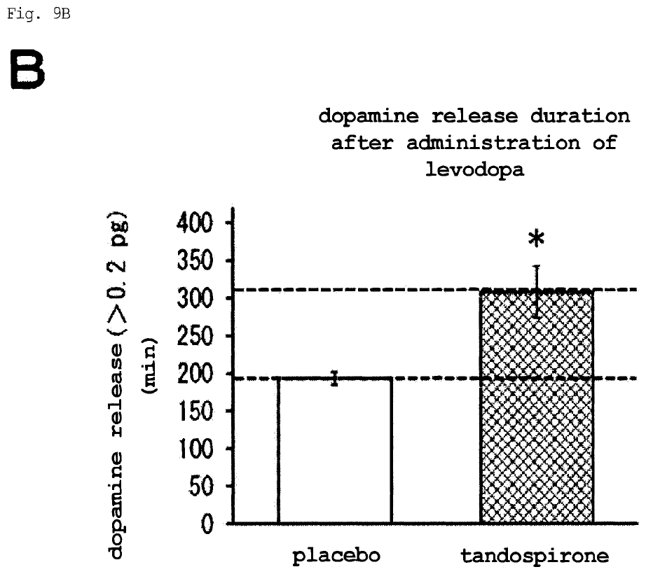

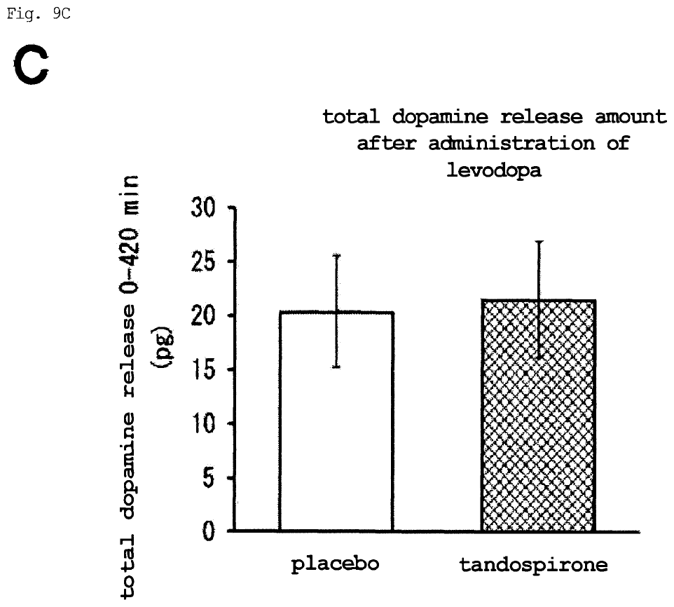

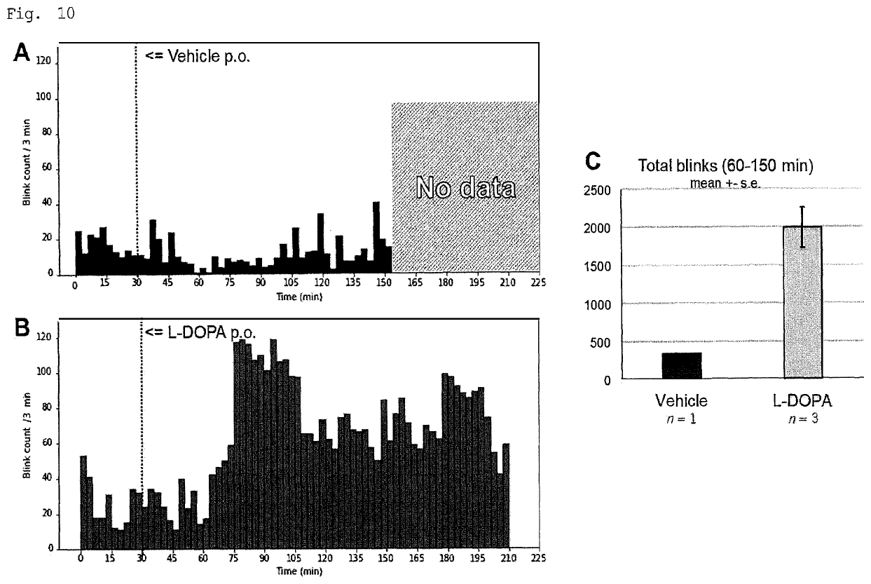

The dopamine change (pg) was measured at each sampling point (every 10 minutes), calculated as the amount of change from the baseline (mean value of 4 samples before administration of the levodopa blended liquid), and measured up to 420 minutes after administration of the levodopa blended liquid. A tandospirone tape agent decreased the amount of dopamine release in the striatum from 30 minutes to 150 minutes after levodopa administration, and increased the amount of dopamine release after 150 minutes (FIG. 9A). When the time required for a dopamine release change of 0.2 μg or more to be observed was calculated, a significant prolongation was observed by applying the tandospirone tape agent compared to the placebo tape administration group (FIG. 9B). On the other hand, no difference was observed in the total dopamine release amount after levodopa administration (FIG. 9C).

As described above, it was found that administration of tandospirone suppresses rapid changes and temporal decreases in the dopamine amount in the synaptic cleft in the striatum.

### (Creation of MPTP-Induced Parkinson's Disease Levodopa-Induced Dyskinesia (PD-LID) Model Rhesus Monkey and Measurement of Eye Movements)

1-Methyl-4-phenyl-1,2,3,6-tetrahydropyridine (MPTP) is a neurotoxin that specifically destroys dopamine neurons. Model animals in which dopaminergic neurons projecting from the substantia nigra to the striatum are destroyed by intravenous administration, intramuscular administration, or surgical administration into the internal carotid artery of MPTP are widely used to evaluate the drug efficacy of therapeutic agents for Parkinson's disease. On the other hand, repeated administration of levodopa to this model is known to induce dyskinesia, which is similar to the clinical long-term administration of levodopa that causes dyskinesia in patients with Parkinson's disease. Using this model, the action of dopamine-related substances on eye movements was evaluated. In this test, male rhesus monkeys (HAMRI CO., LTD.) were used. MPTP (0.4 or 0.6 mg/kg) was administered once or twice weekly and the administration was continued until Parkinson's disease symptoms developed steadily. Thereafter, levodopa (20 or 30 mg/kg) was administered once or twice weekly and the administration was continued until dyskinesias developed steadily.

Using this model monkeys, eye movements were measured. An eye tracking headset Pupil Core manufactured by Pupil Labs was used for eye movement measurement. This device is an eye tracker worn on the face like eye glasses. In this device, an LED attached near the eye irradiates the eyeball with infrared rays, a video of the irradiated eyeball is captured with a high-speed camera of 120 to 200 Hz, and the video is recorded in a PC. The LED lighting and camera unit of this device were transferred to a spectacle-shaped frame made of ABS fabricated according to the head size of the rhesus monkey.

With the model monkey seated on a monkey chair, the monkey eye-tracking headset prepared as described above was worn, and eyeball images were measured. The obtained eyeball images were converted into time-series data of ellipse fitting accuracy as an indicator for pupil coordinates and pupil detection using the analysis software Pupil Player (Version 2.4.0) manufactured by Pupil Labs, then, were analyzed using a self-made program created using Python 3.5. The ellipse fitting accuracy, which is the threshold for closed eyes or open eyes, was set to 0.5. An eye-closing event was considered when the ellipse fitting accuracy transitioned from 0.5 or more to less than 0.5, and an eye-opening event was considered when the ellipse fitting accuracy transitioned from less than 0.5 to 0.5 or more. Segments in which the elapsed time from the eye-closing event to the eye-opening event was 0.05 s to 0.80 s were extracted as blink.

### Reference Example 2: Blink-Increasing Action of L-DOPA in MPTP-Induced PD-LID Rhesus Monkeys

(Test method)

The aforementioned model monkey was seated on a monkey chair and fitted with an eye-tracking headset. After 30 minutes, a vehicle (0.5% methylcellulose 400 aqueous solution, Nacalai Tesque) or L-DOPA/Benserazide suspended in a vehicle (L-DOPA is 22 mg/kg, Benserazide is 1/4 weight of L-DOPA) was orally administered, and eye movements were measured for 120 to 180 minutes after administration.

### (Result)

When the number of blinks was tallied every 3 minutes, there was no significant change in the number of blinks throughout the measurement with vehicle administration (FIG. 10A), but L-DOPA increased the number of blinks from about 30 minutes after administration, and a peak was reached at 30 to 100 minutes after administration (FIG. 10B). For a certain individual, when the number of blinks for 60 to 150 minutes after the start of measurement was tallied for one time for vehicle administration and three times for L-DOPA administration, the L-DOPA-administered group showed a clear tendency to increase the number of blinks compared to the vehicle case (FIG. 10C).

### Reference Example 3: Fluctuation in the Number of Blinks Per Duration During L-DOPA Administration

### (Test Method)

In the reference example described above, the model monkey was seated on a monkey chair and fitted with an eye-tracking headset. After 30 minutes, L-DOPA/Benserazide (L-DOPA is 22 mg/kg, Benserazide is 1/4 weight of L-DOPA) suspended in a vehicle (0.5% methylcellulose 400 aqueous solution, Nacalai Tesque) was orally administered, and eye movements were measured for 180 minutes after administration.

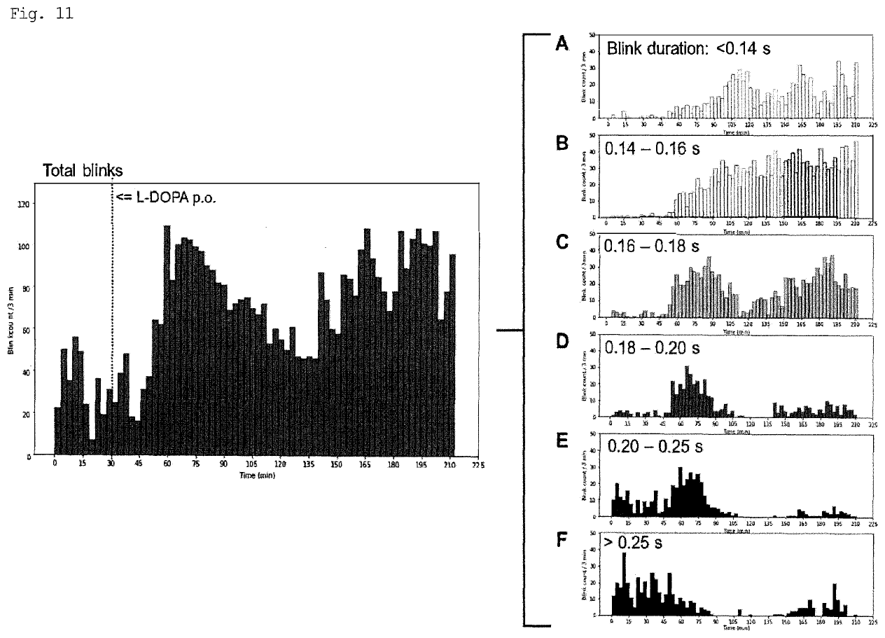

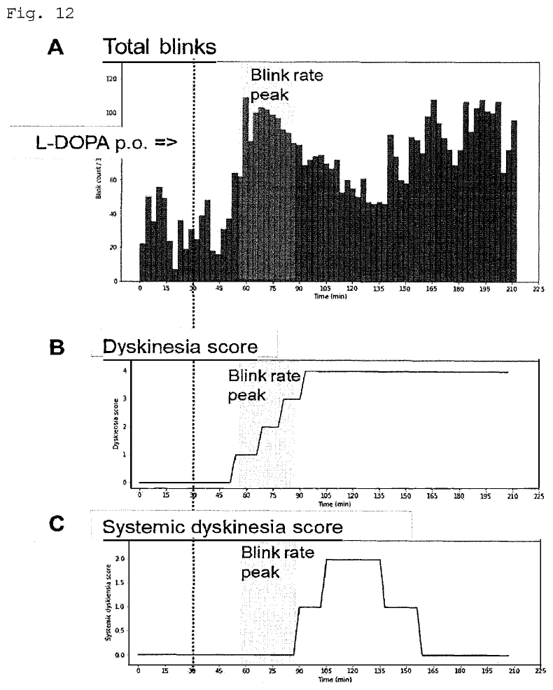

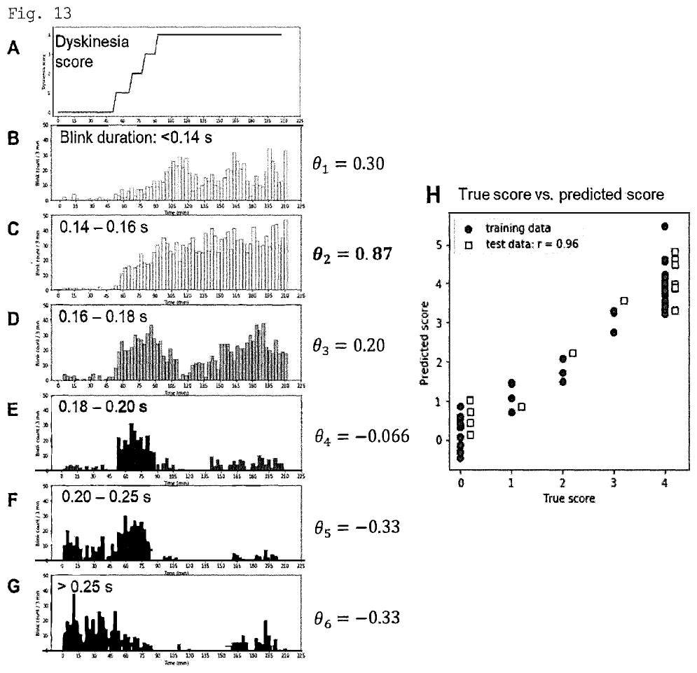

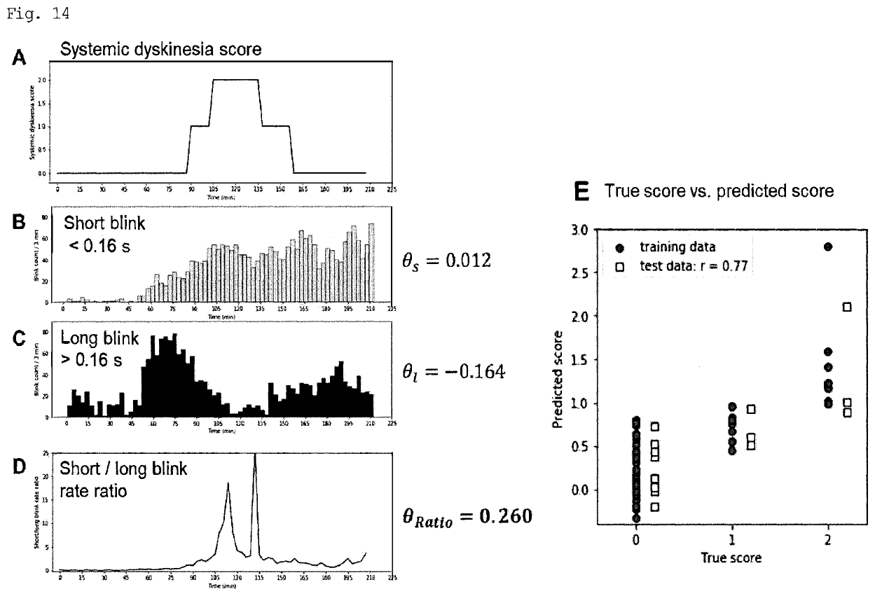

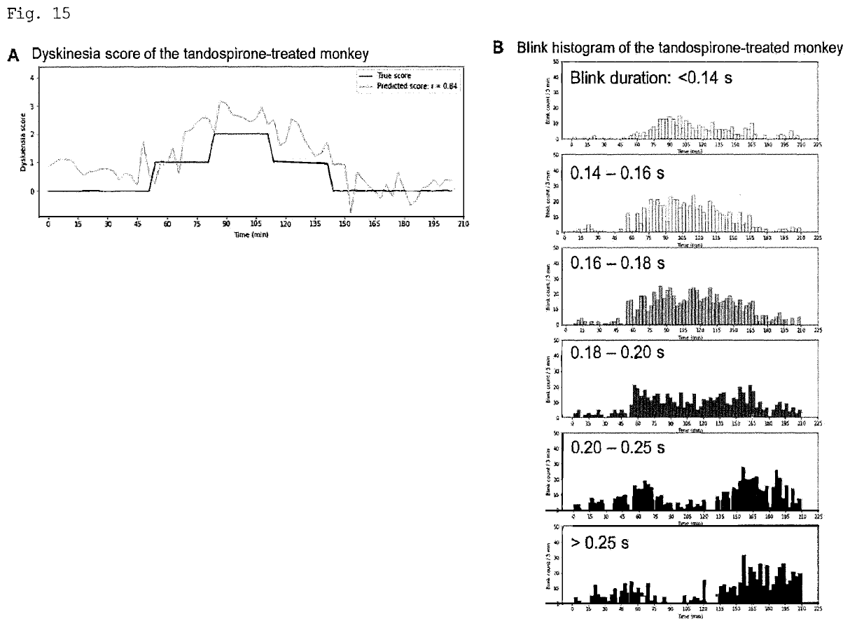

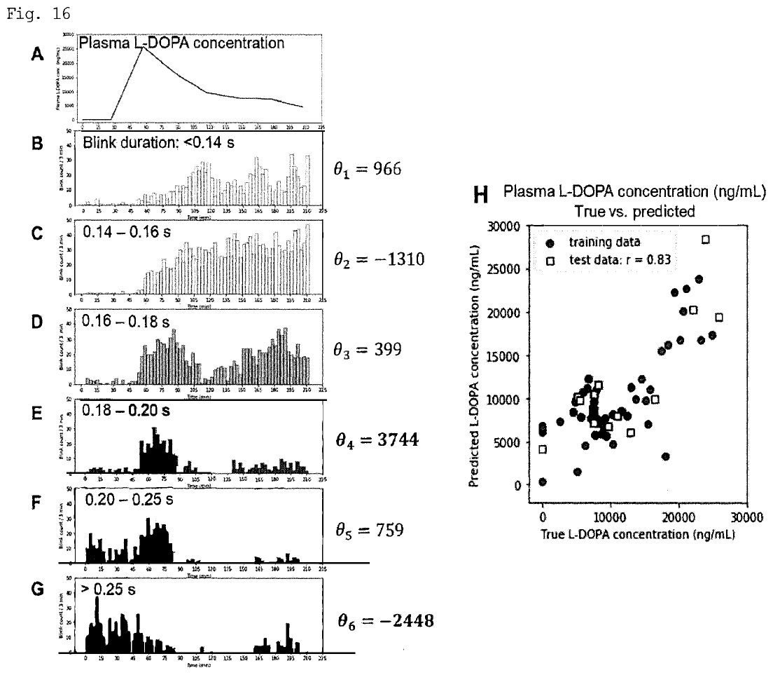

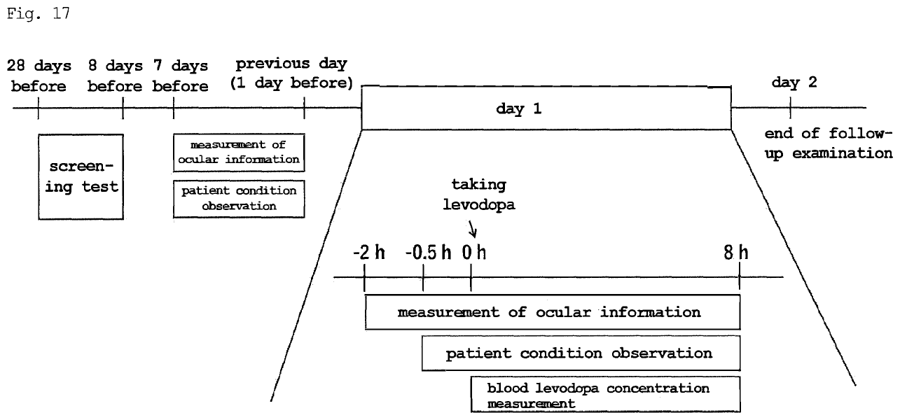

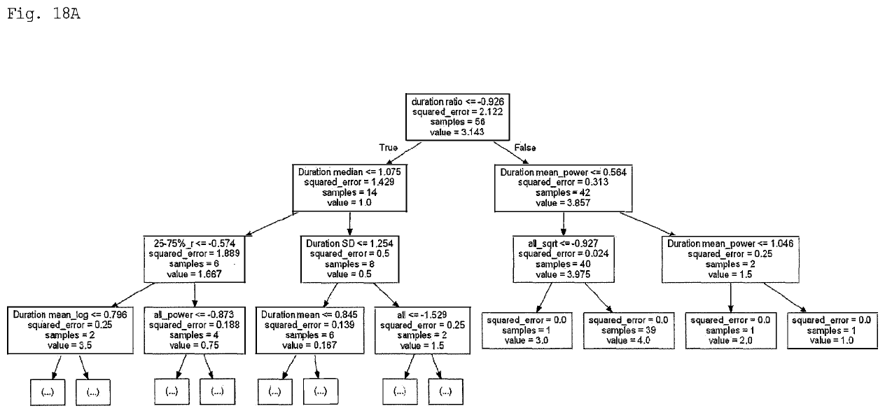

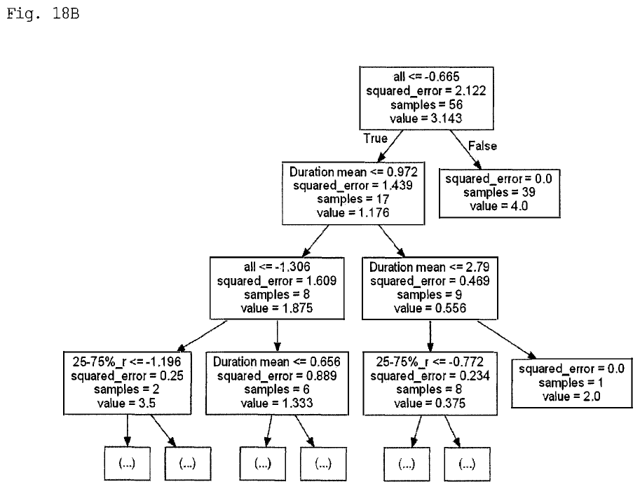

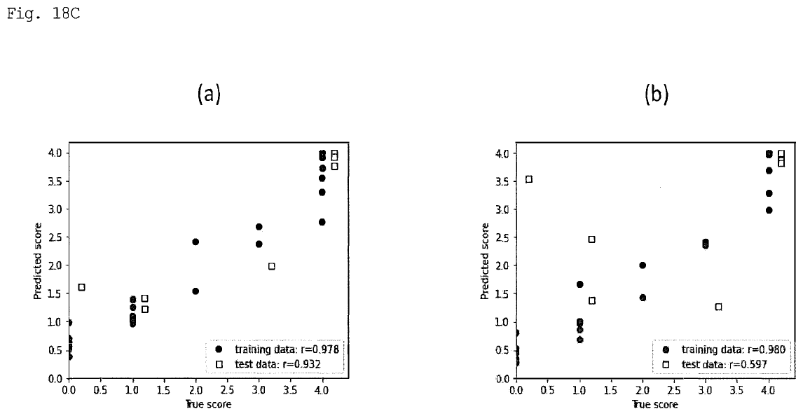

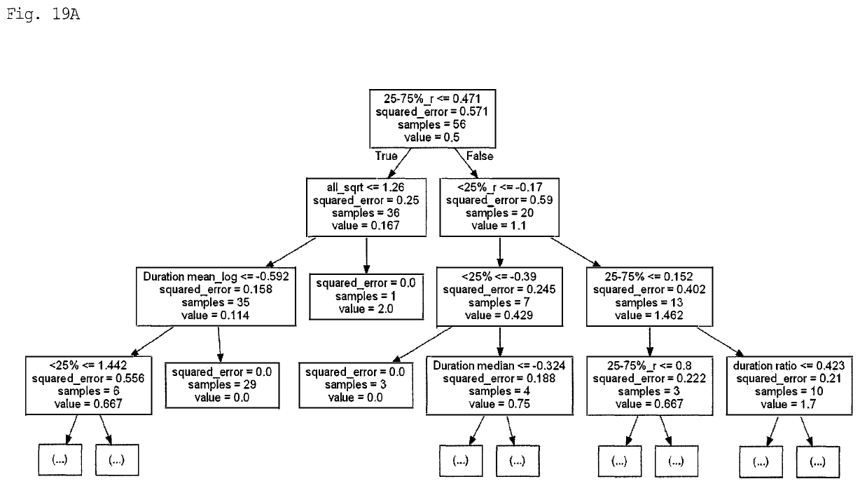

The blinks were classified into 0.05≤T<0.14, 0.14≤T<0.16, 0.16≤T<0.18, 0.18≤T<0.20, 0.20≤T<0.25, and 0.25 T<0.80, depending on its duration T(s), and tallied every 3 minutes (FIGS. 11A to 11F).

### (Result)

In a certain example, the number of blinks with a relatively long duration (0.18≤T<0.25) peaked around 30 minutes after administration, but declined rapidly thereafter (FIGS. 11D and 11E). In addition, the number of blinks with a relatively short duration (T<0.16) gradually increased from 30 minutes after administration to the end of measurement (FIGS. 11A and 11B). In addition, the number of blinks with an intermediate duration (0.16≤T<0.18) showed a peak around 30 minutes after administration and a rapid decrease, while, after the decrease, there was an intermediate trend of a gradual increase until the end of the measurement (FIG. 11C).

### Reference Example 4: Relationship Between Dyskinesia and Blink Frequency During Administration of L-DOPA

### (Test Method)

In the reference example described above, the model monkey was seated on a monkey chair and fitted with an eye-tracking headset. After 30 minutes, L-DOPA/Benserazide (L-DOPA is 22 mg/kg, Benserazide is 1/4 weight of L-DOPA) suspended in a solvent (0.5% methylcellulose 400 aqueous solution, Nacalai Tesque) was orally administered, and eye movements were measured for 180 minutes after administration.

Evaluation of dyskinesias was scored by an evaluator trained in behavioral assessment based on video recordings. The dyskinesia score was evaluated based on the Revised non-human primate dyskinesia rating scale (J Neurosci 2001; 21: 6853-6861.). A score of 0 was given when no dyskinesias was observed; if dyskinesia was observed for less than 30% of the evaluation time, it was considered to be mild dyskinesia and the score was 1; if dyskinesia was observed for 30% or more of the evaluation time but normal behavior was not inhibited, it was considered to be moderate dyskinesia and the score was 2; if dyskinesia was observed for less than 70% and 30% or more of the evaluation time and normal behavior was inhibited, it was regarded as significant dyskinesia and the score was 3; and if dyskinesia was observed for 70% or more of the evaluation time and normal behavior was inhibited, it was regarded as severe dyskinesia and was given a score of 4. In addition, generalized dyskinesia was evaluated as dyskinesia with particularly high severity. With reference to the UdysRS which is a clinical evaluation scale that incorporates dyskinesia by site into the evaluation, if dyskinesia occurred in 4 or more of the 6 areas of the face, right arm, left arm, trunk, right leg, and left leg, it was defined as generalized dyskinesia, and a score of 1 was given if generalized dyskinesia occurred in 30% or more of the evaluation time and a score of 2 was given if generalized dyskinesia occurred 70% or more of the evaluation time.

### (Result)

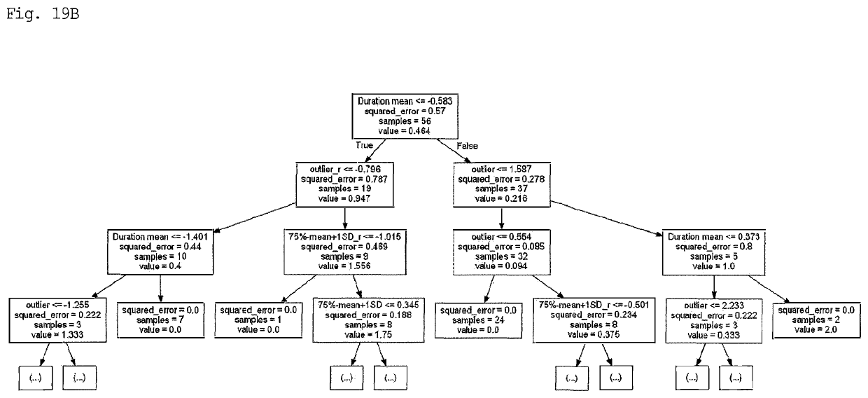

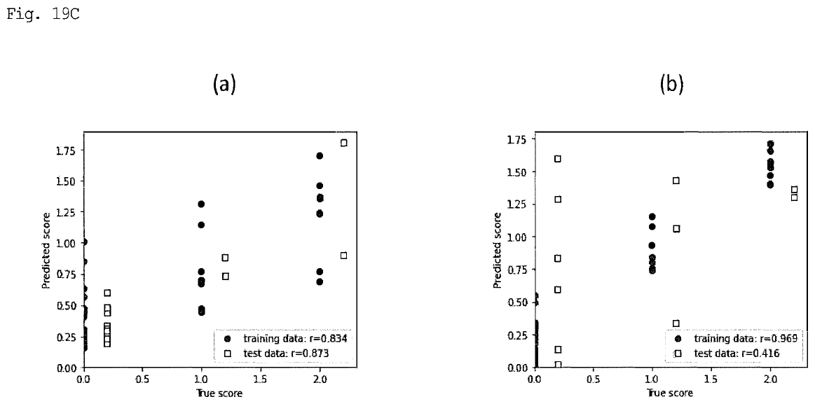

The blink frequency peaked around 30 to 60 minutes after administration of L-DOPA (FIG. 12A). However, the dyskinesia score began to increase at the peak of blinking and peaked after passing the peak of blinking (FIG. 12B). This trend was similar for the scores of generalized dyskinesias, and the delay to the blink score was even more pronounced (FIG. 12C). Considering that the dopaminergic function in the brain is overactivated in dyskinesias, these cannot be explained by the knowledge that blink frequency simply shows a positive correlation with dopamine function in the brain, as previously reported, suggesting that dyskinesia cannot be detected simply by measuring the blink frequency alone.

### (Example 1: Prediction of Dyskinesia Score (1) Based on Ocular Information)

### (Test Method)

The aforementioned model monkey was seated on a monkey chair and fitted with an eye-tracking headset. After 30 minutes, L-DOPA/Benserazide (L-DOPA is 22 mg/kg, Benserazide is 1/4 weight of L-DOPA) suspended in a vehicle (0.5% methylcellulose 400 aqueous solution, Nacalai Tesque) was orally administered, and eye movements were measured for 180 minutes after administration.

The model monkeys transdermally received an ointment containing tandospirone or a placebo ointment without tandospirone. Tandospirone has been reported to exhibit anti-dyskinesia effects by suppressing the release of dopamine in the brain. The backs of the rhesus monkeys were shaved, and the ointment was applied to a 4 cm×10 cm area 19 hours prior to testing, covered with a tape and a clean cloth, and jacketed.

Dyskinesia was evaluated by analyzing videos of model monkeys and scoring them by evaluators skilled in behavioral evaluation. The dyskinesia score was evaluated based on the Revised non-human primate dyskinesia rating scale (J Neurosci 2001; 21: 6853-6861). A score of 0 was given if no dyskinesias was observed; if dyskinesia was observed for less than 30% of the evaluation time, it was considered to be mild dyskinesia and the score was 1; if dyskinesia was observed for 30% or more of the evaluation time but normal behavior was not inhibited, it was considered to be moderate dyskinesia and the score was 2; if dyskinesia was observed for less than 70% and 30% or more of the evaluation time and normal behavior was inhibited, it was regarded as significant dyskinesia and the score was 3; and if dyskinesia was observed for 70% or more of the evaluation time and normal behavior was inhibited, it was regarded as severe dyskinesia and was given a score of 4. In addition, generalized dyskinesia was evaluated as a particularly severe dyskinesia. With reference to the UdysRS which is a clinical evaluation scale that incorporates dyskinesia by site into the evaluation, if dyskinesia occurred in 4 or more of the 6 areas of the face, right arm, left arm, trunk, right leg, and left leg, it was defined as generalized dyskinesia, and a score of 1 was given if generalized dyskinesia occurred in 30% or more of the evaluation time and a score of 2 was given if generalized dyskinesia occurred 70% or more of the evaluation time.

A linear regression model was used for score prediction of dyskinesia and generalized dyskinesia. The predicted value h by a linear regression model is generally expressed as follows, as a formula obtained by adding a bias parameter θ₀ to the linear sum of n features (x₁, x₂, . . . , xₙ) and n parameters (θ₁, θ₂, . . . θₙ).

$${h}_{θ} ( x ) = {\sum }_{j - 1}^{n} {θ}_{j} {x}_{i} + {θ}_{D} \qquad [ Mathematical Formula 1 ]$$

The loss function J of the linear regression model can be expressed as follows when the correct value is y. A search was made for a combination of parameters θ that would minimize the loss function J.

$$J ( θ ) = \frac{1}{2} {( {h}_{θ} ( x ) - y )}^{2} \qquad [ Mathematical Formula 2 ]$$

One data point was defined as a mutually independent 3-minute time window in the measurement. The dyskinesia score for the 3 minutes was set as the correct value y. As the feature x, the blinks are classified into x₁ to x₆ as follows according to the duration T(s), and tallied for each time window to obtain the feature.
x₁: 0.05≤T<0.14, x₂: 0.14≤T<0.16, x₃: 0.16≤T<0.18, x₄: 0.18≤T<0.20, x₅: 0.20≤T<0.25, x₆: 0.25≤T<0.80

Furthermore, in estimating the generalized dyskinesia score, in addition to the feature, the total number of blinks xₜₒₜₐₗ in the corresponding time window, the short blink x₈ with a blink duration T<0.16, and the long blink x₁ with T≥0.16 were added as new features. In addition, a ratio x_{Ratio} obtained by dividing the number of times xₛ of short blinks by the number of times x₁ of long blinks in the same time window was used as an additional feature.

Eighty percent (56 points) of the data points (n=70) obtained from L-DOPA-administered and non-tandospirone-administered individuals were used to train the model. The performance evaluation of the model was performed using the remaining 20% data points (14 points) as test data. A correlation coefficient r between the predicted value and the correct value for the test data was calculated and used as a measure of the prediction performance of the trained model.

To predict the dyskinesia score in tandospirone administration, a model was trained using all the data from the time of non-administration of tandospirone, and the predicted value was output by inputting the features of the time of administration of tandospirone into the trained model.

### (Result)

For individuals with L-DOPA administration and tandospirone no-administration, the dyskinesia score h(x) every 3 minutes and blink frequency divided by the blink duration (features x₁ to x₆) and the corresponding trained parameters θ₁ to θ₆ are shown in FIGS. 13A to 13G. That is, the dyskinesia score h(x) for any 3 minutes in this individual is estimated by the following formula.

h(x)=0.30x₁+0.87x₂+0.20x₃−0.066x₄−0.33x₅−0.33x₆+2.625 [Mathematical Formula 3]

This teaches that a blink with a relatively short duration of feature x₂ is mentioned as a parameter that best explains the dyskinesia score, then, long-duration blinks of x₅ and x₆ are mentioned as parameters that relatively strongly explain the low dyskinesia score. Furthermore, in this model, the correlation coefficient between the predicted value and the correct value at 20 points of test data was calculated to be r=0.96, indicating a very strong correlation (FIG. 13H). Therefore, it was suggested that these features relating to blinking are very useful in predicting the dyskinesia score.

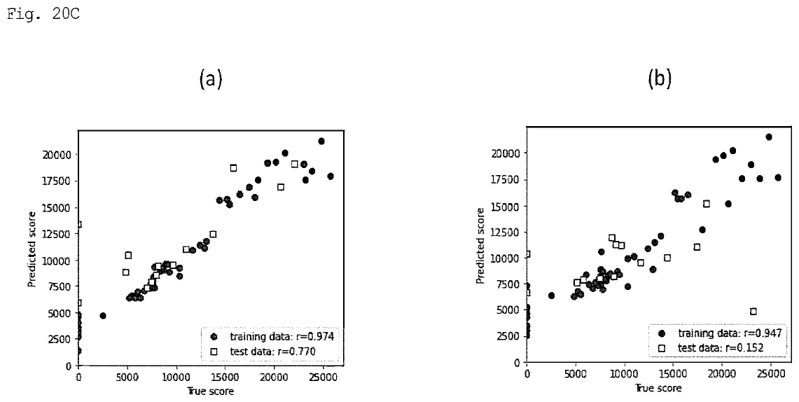

Furthermore, the generalized dyskinesia score h(x) every 3 minutes in the same individual, the number of previous blinks (feature xₜₒₜₐₗ), the frequency of short blinks (feature xₛ), the frequency of long blinks (x₁), the ratio of short and long blinks (x_{Ratio}), and the corresponding trained parameters θₛ, θ₁, and e_{Ratio} are shown in FIGS. 14A to 14D. That is, the generalized dyskinesia score h(x) for any 3 minutes in this individual is estimated by the following formula.

h(x)=−0.063x₁+0.057x₂−0.15x₃+0.020x₄−0.020x₅−0.24x₆−0.015xₜₒₜₐₗ+0.012xₛ−0.16x₁+0.26x_{Ratio} [Mathematical Formula 4]

This teaches that the parameter that best describes the generalized dyskinesia score is x_{Ratio}, the ratio of short to long blinks, and in addition, x₆, which is a relatively long-duration blink, is a parameter that suggests a low generalized dyskinesia score. Furthermore, in this model, when the correlation coefficient between the predicted value and the correct value for 20 test data points is calculated, it becomes r=0.77, and it can be said that there is a positive correlation (FIG. 14E). These parameters were suggested to be useful in estimating severe dyskinesias.

Furthermore, the blink data for L-DOPA-administered and tandospirone-administered individual was input into the trained model obtained by using both blink data and dyskinesia score obtained from the L-DOPA-administered but non-tandospirone-administered individual for model training, and prediction of the dyskinesia score was dried, to obtain a correlation coefficient of r=0.84, showing strong correlation (FIGS. 15A and 15B). From these results, it can be said that this pre-trained model based on blink parameters was able to detect a decrease in dyskinesia score due to administration of tandospirone, suggesting that it is useful for evaluating the drug efficacy of therapeutic agents for Parkinson's disease.

### (Example 1′: Prediction of Dyskinesia Score Based on Ocular Information by Another Method (1))

### (Test Method)

Using the same measurement data obtained in Example 1, the scores of dyskinesia and generalized dyskinesia were predicted by a different method. Predictions were made by random forest.

Regression by random forest model is based on majority decision of multiple decision trees. In each decision tree, the mean squared error (MSE) for each node t is defined as follows, and a decision tree is created to minimize them.

$$MSE ( t ) = \frac{1}{{N}_{t}} {\sum }_{t ∈ {D}_{t}} {( {y}^{( i )} - \hat{{y}_{t}} )}^{2} . \qquad [ Mathematical Formula 5 - 1 ]$$

where, Nₜ represents the number of training data,

y⁽ⁱ⁾ [Mathematical Formula 5-2]
represents the correct answer for the training data, and

yᵢ [Mathematical Formula 5-3]
represents the mean of the aggregation Dt included in the decision tree splitting condition t.

One data point was defined as a mutually independent 3-minute time window in the measurement. The input value was set to the blink duration during the three minutes and a parameter derived from the blink duration, and the output value was set to the score during the three minutes. Parameters derived from blink duration included ratio of short to long blinks, median blink duration, square value of the mean value of the blink duration, proportion of blinks whose blink duration is shorter than their first quartile, number of blinks whose blink duration is less than their first quartile, ratio of blinks whose blink duration is between their 1st and 3rd quartiles, number of blinks whose blink duration is between their first and third quartiles, standard deviation of blink duration, square root value of number of blinks, logarithm of mean blink duration, square value of number of blinks, and mean blink duration. Here, the ratio of short blinks to long blinks is the ratio of the number of short blinks with a blink duration T<0.16 divided by the number of long blinks with T≥0.16.

Eighty percent (56 points) of the data points (n=70) obtained from L-DOPA-administered and non-tandospirone-administered individuals were used as training data to construct the random forest model. Using the remaining 20% data points (14 points) as test data, the performance of the random forest model was evaluated. A correlation coefficient r between the predicted value and the correct value for the test data was calculated and used as a measure of the prediction performance of the random forest model.

FIG. 18A shows an example of a decision tree constructing random forest model constructed to predict dyskinesia scores.

Here, duration ratio is the ratio between short and long blinks, duration median is the median blink duration, duration mean_power is the square of the mean blink duration, 25-75%_r is the ratio of blinks whose blink duration is between their 1st and 3rd quartiles, duration_SD is the standard deviation of blink duration, all_sqrt is the square root value of the number of blinks, duration_mean_log is the logarithm of the mean blink duration, all_power is the squared value of the number of blinks, duration_mean is the mean blink duration, and all is the number of blinks.

FIG. 18B shows another example of a decision tree constructing random forest model constructed to predict dyskinesia scores. In this example, total number of blinks, the number of blinks whose blink duration is between the first and third quartile, ratio of blinks whose blink duration is between their first and third quartiles, and mean blink duration were utilized.

Here, all is the number of blinks, 25-75% is the number of blinks whose blink duration is between their first and third quartiles, and 25-75%_r is the ratio of blinks whose blink duration is between their first and third quartiles, and Duration_mean is the mean blink duration.

The test data was input into the constructed random forest model and the dyskinesia score was predicted.

FIG. 18C shows the results of prediction by a random forest model constructed to predict dyskinesia score.

FIG. 18C(a) shows the results from the random forest model with the decision trees shown in FIG. 18A, and FIG. 18C(b) shows the results from the random forest model with the decision trees shown in FIG. 18B.

In FIGS. 18C(a) and 18C(b), ● indicates the result when training data is input to the random forest model, and □ indicates the result when test data is input to the random forest model.

In FIG. 18C(a), when the training data was input to the random forest model, the correlation coefficient r between the predicted value and the correct value was 0.978. When the test data was input to the random forest model, the correlation coefficient r between the predicted value and the correct value was 0.932. A high correlation coefficient is also obtained for the test data. On the other hand, in FIG. 18C(b), when the training data was input to the random forest model, the correlation coefficient r between the predicted value and the correct value was 0.980. However, when the test data was input to the random forest model, the correlation coefficient r between the predicted value and the correct value was 0.597, indicating insufficient prediction accuracy. It can be said that these things suggest that the parameters (that is, Duration ratio, Duration median, Duration_mean_power, 25-75%_r, Duration_SD, all_sqrt, Duration_mean_log, all_power, Duration_mean, all) used in the random forest model of FIG. 18A are useful parameters for predicting the dyskinesia score compared to other parameters (e.g., the combination of parameters used in the random forest model of FIG. 18B).

FIG. 19A shows an example of a decision tree constructing random forest model constructed to predict generalized dyskinesia score.

Here, <25%_r is the ratio of blinks whose blink duration is shorter than their first quartile, duration ratio is the ratio between short and long blinks, 25-75% is the number of blinks whose blink duration is between the first and third quartiles, 25-75%_r is the ratio of blinks whose blink duration is between their 1st and 3rd quartiles, <25% is the number of blinks whose blink duration is less than their first quartile, all_sqrt is the square root value of the number of blinks, duration median is the median blink duration, and duration_mean_log is the logarithm of the mean blink duration.

FIG. 19B shows another example of a decision tree constructing random forest model constructed to predict generalized dyskinesia score. In this example, number of blinks whose blink duration is greater than or equal to their third quartile and less than mean+SD, ratio of blinks whose blink duration is greater than or equal to their third quartile and less than mean+SD, the number of blinks whose blink duration is greater than or equal to their mean+SD, ratio of blinks whose blink duration is greater than or equal to their mean+SD, and mean blink duration were used.

Here, 75%-mean+1SD is the number of blinks whose blink duration is greater than or equal to their third quartile and less than their mean+SD, 75%-mean+1SD_r is the ratio of blinks whose blink duration is greater than or equal to the third quartile and less than the mean+SD, outlier is the number of blinks whose blink duration is greater than or equal to their mean+SD, outlier_r is the ratio of blinks whose blink duration is greater than or equal to their mean+SD, and Duration_mean is the mean blink duration.

The test data was input into the constructed random forest model and the systemic dyskinesia score was predicted.

FIG. 19C shows the results of prediction by a random forest model constructed to predict generalized dyskinesia score.

FIG. 19C(a) shows the results from the random forest model with the decision trees shown in FIG. 19A, and FIG. 19C(b) shows the results from the random forest model with the decision trees shown in FIG. 19B.

In FIGS. 19C(a) and 19C(b), ● indicates the result when training data is input to the random forest model, and Q indicates the result when test data is input to the random forest model.

In FIG. 19C(a), when the training data was input to the random forest model, the correlation coefficient r between the predicted value and the correct value was 0.834. When the test data was input to the random forest model, the correlation coefficient r between the predicted value and the correct value was 0.873. A high correlation coefficient is also obtained for the test data. On the other hand, in FIG. 19C(b), when the training data was input to the random forest model, the correlation coefficient r between the predicted value and the correct value was 0.969. However, when the test data was input to the random forest model, the correlation coefficient r between the predicted value and the correct value was 0.416, indicating low prediction accuracy. It can be said that these things suggest that the parameters (that is, <25%_r, Duration ratio, 25-75%, 25-75%_r, <25%, all_sgrt, Duration median, Duration_mean_log) used in the random forest model of FIG. 19A are useful parameters for predicting the dyskinesia score compared to other parameters (e.g., the combination of parameters used in the random forest model of FIG. 19B).

### (Example 2: Prediction of Dyskinesia Score (2) Based on Ocular Information)

In this example, the dyskinesia score was predicted based on eyeball information other than blinking.

### (Method)

The severity of dyskinesia at the time of measurement is estimated using information on eye movement as eyeball information other than blinking. Taking advantage of the fact that ocular tremor increases when the severity of dyskinesia is high, frequency analysis of pupillary coordinates, eye movement distance, and eye movement speed was performed to obtain characteristic spectral bands, and a prediction of the dyskinesia score is made from the correlation with the severity of dyskinesia.

In addition, eyelid opening information is used as eyeball information other than blinking to estimate the severity of dyskinesia at the time of measurement. Utilizing the fact that the eyelids droop and vibrate irregularly in cases of high dyskinesia severity, the dyskinesia severity is estimated from decreased eyelid opening and increased variance.

### Example 3: Predict L-DOPA Concentration in Peripheral Blood Based on Eyeball Information

### (Test Method)

The aforementioned model monkey was seated on a monkey chair and fitted with an eye-tracking headset. After 30 minutes, L-DOPA/Benserazide (L-DOPA is 22 mg/kg, Benserazide is ¼ weight of L-DOPA) suspended in a vehicle (0.5% methylcellulose 400 aqueous solution, Nacalai Tesque) was orally administered, and eye movements were measured for 180 minutes after administration.

Blood was collected from the upper arm of the model monkey every 30 minutes, and L-DOPA concentration in plasma was quantified using an HPLC system.

A linear regression model was used to predict plasma L-DOPA concentrations. One data point was defined as a mutually independent 3-minute time window in the measurement. The correct value y was set to the plasma L-DOPA concentration (ng/mL) for 3 minutes, which is predicted by linearly interpolating the L-DOPA measurement result every 30 minutes. As the feature x, the blinks are classified into x₁ to x₆ as follows according to the duration T(s), and tallied for each time window to obtain the feature. x₁: 0.05≤T<0.14, x₂: 0.14≤T<0.16, x₃: 0.16≤T<0.18, x₄: 0.18≤T<0.20, x₅: 0.20≤T<0.25, x₆: 0.25≤T<0.80

For model training, 80% (56 points) of the data points (n=70) obtained from the L-DOPA-administered model monkeys were used. The performance evaluation of the model was performed using the remaining 20% data points (14 points) as test data. A correlation coefficient r between the predicted value and the correct value for the test data was calculated and used as a measure of the predicted performance of the trained model.

### (Result)

For L-DOPA-administered model monkeys, plasma L-DOPA concentration h(x) every 3 minutes and blink frequency divided by blink duration (features x₁ to x₆), and the corresponding trained parameters θ₁ to θ₆ are shown in FIGS. 16A to 16G. That is, the plasma L-DOPA concentration h(x) (ng/mL) in an arbitrary 3-minute period in this individual is estimated by the following formula.

h(x)=966x₁−1310x₂+399x₃+3744x₄+759x₅−2448x₆+9414 [Mathematical Formula 6]

This teaches that an intermediate blink with a duration of feature x₄ is mentioned as a parameter that best describes the plasma L-DOPA concentration, then, a long-duration blink of x₆ is mentioned as a parameter that relatively strongly explains the low plasma L-DOPA concentration. Furthermore, in this model, the correlation coefficient between the predicted value and the correct value for 20 test data points was calculated to be r=0.83, indicating a strong correlation (FIG. 16H). Therefore, it was suggested that these features related to blinking are useful in predicting the plasma L-DOPA concentration.

### Example 3′: Predicting L-DOPA Concentration in Peripheral Blood Based on Eyeball Information by Another Method

### (Test Method)

Using the same measurement data obtained in Example 3, the L-DOPA concentration in peripheral blood was predicted by another method. Predictions were made by random forest.

Regression by random forest model is based on majority decision of multiple decision trees. In each decision tree, the mean squared error (MSE) for each node t is defined as follows, and a decision tree is created to minimize them.

$$MSE ( t ) = \frac{1}{{N}_{t}} {\sum }_{t ∈ {D}_{t}} {( {y}^{( i )} - \hat{{y}_{t}} )}^{2} . \qquad [ Mathematical Formula 7 - 1 ]$$

where, Nₜ represents the number of training data,

y⁽ⁱ⁾ [Mathematical Formula 7-2]
represents the correct answer for the training data, and

\acute{y}ᵢ [Mathematical Formula 7-3]
represents the mean of the subset Dt included in the decision tree splitting condition t.

One data point was defined as a mutually independent 3-minute time window in the measurement. The input value was set to the blink duration in the three minutes and a parameter derived from the blink duration, and the output value was set to the plasma L-DOPA concentration (ng/mL) for 3 minutes, which is predicted by linearly interpolating the L-DOPA measurement result every 30 minutes. The parameters derived from the blink duration included the ratio of short to long blinks, the median blink duration, the square of the mean blink duration, the ratio of blinks whose blink duration is less than their first quartile, the number of blinks whose blink duration is less than their first quartile, the ratio of blinks whose blink duration is between their first and third quartiles, the number of blinks whose blink duration is between the first and third quartile, the standard deviation of blink duration, the square root value of number of blinks, the logarithm of mean blink duration, the square value of the number of blinks, and the mean blink duration
Here, the ratio of short blinks to long blinks is the ratio of the number of short blinks with a blink duration T<0.16 divided by the number of long blinks with T≥0.16.

Eighty percent (56 points) of the data points (n=70) obtained from the L-DOPA-administered model monkeys were used to construct the random forest model. The remaining 20% of the data points (14 points) were used as test data to evaluate the performance of the random forest decision tree. A correlation coefficient r between the predicted value and the correct value for the test data was calculated and used as a measure of the prediction performance of the random forest model.

FIG. 20A shows an example of a decision tree constructing a random forest model constructed to predict L-DOPA concentration in peripheral blood.

Here, Duration ratio is the ratio of short to long blinks, <25%_r is the ratio of blinks whose blink duration is shorter than their first quartile, Duration median is the median blink duration, 25-75%_r is the ratio of blinks whose blink duration is between their first and third quartiles, 25-75% is the number of blinks whose blink duration is between the first and third quartiles, Duration max is the maximum blink duration, Duration_SD is the standard deviation of blink duration, <25% is the number of blinks whose blink duration is less than its first quartile, all_sqrt is the square root value of the number of blinks, Duration_mean is the mean blink duration, Duration_mean_log is the logarithm of the mean blink duration, and Duration_mean_power is the square of the mean blink duration.

FIG. 20B shows another example of a decision tree constructing random forest model constructed to predict L-DOPA concentrations in peripheral blood. In this example, the total number of blinks, the number of blinks whose blink duration is greater than or equal to their third quartile and less than mean+SD, the ratio of blinks whose blink duration is greater than or equal to their third quartile and less than mean+SD, the number of blinks whose blink duration is greater than or equal to their mean+SD, the ratio of blinks whose blink duration is greater than or equal to their mean+SD, and the mean value of blink duration were utilized.

Here, all is the number of blinks, 75%-mean+1SD is the number of blinks whose blink duration is greater than or equal to their third quartile and less than the mean+SD, 75%-mean+1SD_r is the ratio of blinks whose blink duration is greater than or equal to their third quartile and less than the mean+SD, outlier is the number of blinks whose blink duration is greater than or equal to their mean+SD, outlier_r is the ration of blinks whose blink duration is greater than or equal to their mean+SD, and Duration_mean is the mean blink duration.

Test data was input to the constructed random forest model to predict L-DOPA concentration in peripheral blood.

FIG. 20C shows the results of prediction by a random forest model constructed to predict L-DOPA concentration in peripheral blood.

FIG. 20C(a) shows the results from the random forest model with the decision trees shown in FIG. 20A, and FIG. 20C(b) shows the results from the random forest model with the decision trees shown in FIG. 20B.

In FIGS. 20C(a) and 20C(b), ● indicates the result when training data is input to the random forest model, and □ indicates the result when test data is input to the random forest model.

In FIG. 20C(a), when the training data was input to the random forest model, the correlation coefficient r between the predicted value and the correct value was 0.974. When the test data was input to the random forest model, the correlation coefficient r between the predicted value and the correct value was 0.770. A high correlation coefficient is also obtained for the test data. On the other hand, in FIG. 20C(c), when the training data was input to the random forest model, the correlation coefficient r between the predicted value and the correct value was 0.947. However, when the test data was input to the random forest model, the correlation coefficient r between the predicted value and the correct value was 0.152, indicating extremely low prediction accuracy. It can be said that these things suggest that the parameters (that is, Duration ratio, <25%_r, Duration median, 25-75%_r, 25-75%, Duration max, Duration_SD, <25%, all_sgrt, Duration_mean, Duration_mean_log, Duration_mean_power) used in the random forest model of FIG. 20A are useful parameters for predicting the dyskinesia score compared to other parameters (e.g., the combination of parameters used in the random forest model of FIG. 20B).

(Example 4: Predict the presence/absence, amount or level of L-DOPA, or its variation based on ocular information) in this embodiment, the presence or absence, the amount or level of L-DOPA, or its variation is predicted based on blinking, which is representative of the eyeball information.

The number of blinks per blink time (e.g., >0.25 s, 0.25 s, 0.20 s, 0.16 s, 0.14 s, <0.12 s, etc.) when L-DOPA was administered to patients with Parkinson's disease was measured. A correlation between this measured value and the occurrence of Parkinson's symptoms, motor complications, psychiatric symptoms, increase/decrease in the amount of dopamine, the presence/absence of L-DOPA, fluctuations, etc., is obtained in advance.

The number of blinks per blink time is measured from the subject patient, and the presence/absence, amount or level of L-DOPA, or its variation is predicted from this measured value. As methods of predicting the presence/absence, amount or level of L-DOPA, or its variation in Parkinson's disease, the concentrations of L-DOPA, DA (Dopamine), DOPAC (3,4-dihydroxyphenylacetic acid), HVA (homovanillic acid), 3-OMD (3-O-methyldopa), Noradrenaline and the like in plasma or cerebrospinal fluid after L-DOPA administration are measured, and as methods of measuring L-DOPA in the brain, diagnostic imaging methods using Positron emission tomography (PET), single photon emission computed tomography (SPECT), and the like are mentioned. As diagnostic imaging in the brain, [¹¹C]raclopride, [¹¹C]SCH23390, [¹¹C]2-carbomethoxy-3-(4-fluorophenyl) tropane ([¹¹C]CFT), [¹¹C]dihydro Tetrabenazine ([¹¹C]DTBZ), [¹²³I]N-(3-fluoropropyl)-2β-carbomethoxy-3β-(4-iodophenyl)nortropane ([¹²³I]-FP-CIT), [¹²³I] indobenzamide ([¹²³I] IBZM), 6-[¹⁸F] fluoro-L-DOPA ([¹⁸F] FDOPA), [¹⁸F]fluorodeoxyglucose ([¹⁸F]FDG), and the like can be used.

(Example 5: Predict presence or absence, amount or level of L-DOPA, or its variation based on eyeball information) In this example, the presence/absence, amount or level of L-DOPA, or its variation is predicted based on information on eyeball movement, as eyeball information other than blinking. Taking advantage of the fact that ocular tremor increases when the amount of L-DOPA in vivo is excessive and the severity of dyskinesia is increased, frequency analysis of pupillary coordinates, eye movement distance, and eye movement speed is performed to obtain characteristic spectral bands, thereby predicting the presence or absence, amount or level of L-DOPA in vivo, or its variation. Information on eyelid opening can be used as eyeball information other than blinking. If the amount of L-DOPA is low, the eyelids droop and move poorly. In addition, when the amount of L-DOPA in vivo is excessive and the severity of dyskinesia is high, the eyelids droop and vibrate irregularly. Utilizing these, the presence or absence, amount or level of L-DOPA, or its variation can be predicted based on the decrease in eyelid opening and the increase or decrease in its dispersion.

When L-DOPA is administered to a patient with Parkinson's disease, eyeball information other than blinking, such as frequency analysis spectrum of pupillary coordinates, eyeball movement distance, and eyeball movement speed, and eyelid opening and its dispersion, are measured. Correlations between these measured values and the occurrence of wearing-off or dyskinesia, increase/decrease in the amount of dopamine, presence/absence of L-DOPA, variation, etc., is obtained in advance.

Eyeball information other than blinks such as frequency analysis spectrum of pupillary coordinates, eyeball movement distance, and eyeball movement speed, and eyelid opening and its dispersion are measured from the target patient, and the presence or absence, amount or level of L-DOPA, or its variation are predicted from these measured values. As methods of predicting the presence/absence, amount or level of L-DOPA, or its variation in Parkinson's disease, the concentrations of L-DOPA, DA (Dopamine), DOPAC (3,4-dihydroxyphenylacetic acid), HVA (homovanillic acid), 3-OMD (3-O-methyldopa), Noradrenaline and the like in plasma or cerebrospinal fluid after L-DOPA administration are measured, and as methods of measuring L-DOPA in the brain, diagnostic imaging methods using Positron emission tomography (PET), single photon emission computed tomography (SPECT), and the like are mentioned. As diagnostic imaging in the brain, [¹¹C]raclopride, [¹¹C]SCH23390, [¹¹C]2-carbomethoxy-3-(4-fluorophenyl) tropane ([¹¹C]CFT), [¹¹C]dihydro Tetrabenazine ([¹¹C]DTBZ), [¹²³I]N-(3-fluoropropyl)-2β-carbomethoxy-3β-(4-iodophenyl)nortropane ([¹²³I]-FP-CIT), [¹²³I] indobenzamide ([¹²³I] IBZM), 6-[¹⁸F] fluoro-L-DOPA ([¹⁸F]FDOPA), [¹⁸F]fluorodeoxyglucose ([¹⁸F]FDG) and the like are used in some cases.

### Example 6: Measurement of Striatal Dopamine Concentration in MPTP-Induced Parkinson Model Monkeys

(Preparation of model animals)

In this example, in order to measure striatal dopamine release, an MPTP-induced unilateral Parkinson's disease model was prepared with reference to a method described in a reference (Yamagata University Faculty of Medicine. 2009; 27(2): 119-133). Male cynomolgus monkeys (Shin Nippon Biomedical Laboratories, Ltd.) of 3 to 5 years old were used for preparation of model animals. Atropine sulfate hydrate (0.01 mg/kg; Mitsubishi Tanabe Pharma Corporation) is administered intramuscularly as an anesthetic premedication. Next, ketamine hydrochloride (10 mg/kg; Arevipharma GmbH or Daiichi Sankyo Propharma Co., Ltd.) was intramuscularly administered, and buprenorphine hydrochloride (0.01 mg/kg; Otsuka Pharmaceutical Co., Ltd.) was intramuscularly administered immediately before surgery. Insertion and placement of a catheter (Nippon Becton Dickinson Company, Ltd.) into the left internal carotid artery is performed under inhalation anesthesia of isoflurane (0.5-2%; DS Pharma Animal Health Co., Ltd. or Mylan Inc.). A catheter is inserted into the left internal carotid artery, and the other end is passed subcutaneously to the back and left in place. Two to three days after the placement surgery, MPTP-HCl (0.6 mg/kg; Sigma-Aldrich) is locally administered intra-arterially through the placement catheter under anesthesia with intramuscular administration of ketamine hydrochloride. A second MPTP administration is performed in the same manner after a washout period of 1 to 2 weeks. Improvement of pathological conditions by administration of antiparkinsonian drugs to model animals is confirmed by orally administering L-DOPA (Sigma-Aldrich) and benzelazide hydrochloride (Sigma-Aldrich) and measuring the parkinsonism score.

### (Guide Cannula Placement Surgery in Striatum)

For placement of the guide cannula in the striatum, after induction anesthesia with ketamine hydrochloride, general anesthesia is performed by combined inhalation with a mixed gas of nitrogen and oxygen (2:1) and isoflurane. After the animal's head was fixed in a stereotaxic apparatus, the head skin was incised, the muscle and periosteum were peeled off, the skull was exposed, a contrast medium (Eisai Co., Ltd.) was added to the reference point, and an MRI image is taken and the placement site coordinates are acquired. The guide cannula is fixed to a stereo guide (EiCOM Co., Ltd.) and brought to the coordinates of the placement site. The lumen of the guide cannula is protected, and the suture is removed when healing of the surgical site is confirmed or 2 weeks after the surgery.

### (Microdialysis)

Sampling is performed from before L-DOPA administration to 200 minutes after administration by a microdialysis method, and the amount of dopamine released in the striatum is measured. After induction anesthesia with ketamine hydrochloride, a micro-dialysis probe (EiCOM Co., Ltd.) is inserted into the guide cannula under isoflurane inhalation anesthesia, the tip of the probe is allowed to reach at the target brain coordinates, then, L-DOPA or a control substance is administered while perfusing with an artificial cerebrospinal fluid (Otsuka Pharmaceutical Factory, Inc.) using a syringe pump, and sample collection is performed. The amount of dopamine in the collected dialysate is measured with a fluorescence detector or an electrochemical detector.

### (Example 7) Search for Objective Parameters of Ocular Information Correlated with Parkinson's Symptoms

For Parkinson's disease patients taking levodopa, objective parameters of ocular information that correlates with the symptoms of Parkinson's disease patients are acquired by measuring ocular information before and after taking levodopa, observing the patient's condition, and measuring the blood levodopa concentration. As an example of a clinical test method, after screening Parkinson's disease patients' background information (age, gender, medical history, treatment history, etc.), general condition, Parkinson's symptoms, blood tests, and the like, acclimation to the measurement of ocular information is conducted for about a week, and an influence on the patient's condition is confirmed. After confirming the off state before the start of the test, levodopa is taken, and ocular information is measured, the patient's condition is observed, and blood levodopa concentration is measured over time for up to 8 hours. The patient's condition is observed on the day following the test.

FIG. 17 illustrates the flow of the above test.

### Example 8: Example in Application

As shown in these examples, oculomotor measurements such as blink parameters can be used to estimate in vivo dopamine amount, and in patients with Parkinson's disease, it is possible to estimate objective measures such as dyskinesia severity and the like that are consistent with clinical evaluation. This could be a means of solving the problems of today's Parkinson's disease treatment. Examples are shown below.

### (Patient)

This example demonstrates an application related to Parkinson's disease. As a user of this example, all patients with Parkinson's disease from immediately after being diagnosed with Parkinson's disease to advanced stages and late stages can be targeted. Furthermore, even if not diagnosed with Parkinson's disease, suspected cases of Parkinson's disease and people who are considered to be at high risk of developing Parkinson's disease can be targets. People who are considered to be at high risk of developing Parkinson's disease include, for example, those with mutations in hereditary Parkinson's disease-causing genes (such as SNCA, PARK2, PINK1, etc.), those with olfactory disorders, those with REM sleep behavior disorder, and relatively elderly people aged 60 to 70 or older, and the like.

### (Wearing)

In one embodiment, the user wears smart glasses with an eye movement measurement function, an electrooculogram measurement device, eyeglasses to which an eye movement measurement device is externally attached, or the like, so that it is possible to measure eye movement on a daily basis. In some embodiments, a non-wearable device can also be used, for example, a stationary eye tracking device, a smartphone or a tablet terminal with a camera function, a personal computer, etc., can be used to perform eye movement measurement for a relatively short period of time.

### (Daily Life, Measurement)

Users are expected to obtain the most abundant data by wearing the eye movement measurement device all day long, for example, except when sleeping or bathing, but may wear the device only at certain times of the day, such as 1 to 3 hours after taking the medication after waking up, in order to acquire data relatively stably and continuously. For non-wearable devices, for example, at the patient's home or medical institution, an eye movement measurement can be performed for 3 to 5 minutes at a time using an installed eye tracking device, a smartphone or a tablet terminal with a camera function, a personal computer, etc., and measurements may be carried out about 1-5 times a day to see changes over time. Measurement of eye movement data may be carried out not only on a daily basis but also on a regular basis. As for the frequency of regular measurement, it is most desirable to measure daily, but practically, it is carried out regularly, such as once to three times a week, once every 1 to 3 months, and once every six months to a year, to capture changes over time. The measurement environment is not particularly limited as long as it does not affect the performance of the measuring device, but the user's home, medical institution, regular health checkup, or the like, is assumed.

### (Diagnosis and Administration by Doctor)

The eye movement data acquired in this way is accumulated in the user device and/or server device, and various estimation algorithms are used to output an indicator of the amount of dopamine in the brain, an indicator of the severity of motor symptoms or dyskinesia, and the like.

Such embodiments may be a means of solving the problems of today's treatment of Parkinson's disease.

For example, symptom evaluation of Parkinson's disease patients is done by subjective rating scales of clinicians and patients, such as UdysRS and patient diaries. However, the accuracy of such subjective measures is influenced by many factors, including the clinician's experience and training, the patient's temperament, and the relationship between the clinician and the patient, and results vary among different raters. Subjective rating scales also impose various costs and limitations by requiring clinicians and patients to actively perform assessments. Evaluation by a clinician is usually performed during a visit to the hospital about once every 1 to 3 months, and is merely a measurement at one point in the day. However, Parkinson's disease has large diurnal and inter-day fluctuations, so it is difficult to obtain measured values that truly reflect the patient's condition with such infrequent and short-term evaluations.

In one example of this embodiment, eye movement is applied to provide a new indicator for objectively evaluating the motor symptoms and motor complications of Parkinson's disease and the amount of dopamine in vivo. Although the frequency and time of evaluation may vary depending on the embodiment, in all cases, it provides a means to objectively and easily measure, analyze and report patient status remotely and on a much more frequent and continuous basis than clinical conditions, by which the measurement is convenient for the patient, even in the absence of active symptom monitoring by clinicians and patients. These processes are almost automatic. The output results are automatically converted into a report format and can be used as a clinical tool that clinicians can refer to at will. Objective and continuous data collection allows a more accurate capture of patient symptoms, including diurnal and diurnal variations. With that information, clinicians can more effectively tailor doses and types of medications to reduce the frequency and severity of motor symptoms and complications, such as dyskinesias and on/off symptoms. This will enable precision medicine to deliver treatments optimized for individual symptoms without additional burden on clinicians and patients, improving the patient's quality of life. A broader advantage of this embodiment is that the frequency of in-person visits by patients to medical institutions can be reduced, reducing the financial burden of healthcare on patients, medical institutions and the state, expected to come closer optimizing health economics.

In one example of the present embodiment, by simultaneously referring to peripheral information such as measurement records based on the user's and other users' past eye movements, patient diaries, evaluation records by clinicians, and medication status, and analyzing and reporting, more useful information to prompt clinicians to make better decisions can also be provided, in addition to the above information.

In addition, in one example of this embodiment, when the patient is receiving device-assisted treatment such as deep brain stimulation therapy or L-DOPA preparation transdermal or enteral device administration, it is possible to build a closed-loop system that controls the degree of device treatment in real time in response to fluctuations in symptoms, such as the dosage and administration rate of L-DOPA preparations, and the stimulation frequency and intensity of deep brain stimulation therapy within a safe range, by using the output motor symptoms, motor complications, or estimates of dopamine levels in vivo as input values for device-assisted treatment algorithms.

In one embodiment, some items in the report reporting the patient's condition can be viewed not only by the medical staff but also by the patient, and it is possible to encourage active participation in treatment by visualizing the patient's symptoms and their degree of improvement. Furthermore, it is also possible to provide an alert and reminder function that prompts the patient to take medicine according to deterioration of symptoms including the present, several minutes to several tens of minutes in the future, and the passage of time from taking medicine.

In recent years, an objective evaluation system for Parkinson's disease symptoms using a wristwatch-type acceleration and angular velocity measurement device (IMU) has been studied, but symptoms that can be evaluated are limited to chorea of dyskinesia and resting tremor, etc., and it has only been able to grasp one aspect of the motor symptoms and motor complications of Parkinson's disease, which manifest in various ways. In addition, since the device is powerless against symptoms that do not occur in the arm where the device is worn, it cannot be applied to all patients and does not necessarily lead to a solution to the problem. The present disclosure focuses on eye movements that directly reflect dopamine function in the brain, and is distinct from IMU-based systems that directly quantify motor symptoms and motor complications of Parkinson's disease. Accordingly, in the embodiment according to the present disclosure, changes in brain dopamine function, which is the essence of pathology, can be sensitively captured.

In one example of this embodiment, the progression of Parkinson's disease can also be estimated by measuring in vivo dopamine levels and/or responsiveness to therapeutic agents such as L-DOPA over time using eye movements. Therefore, by periodically performing measurements according to this embodiment for patients who are taking or treating disease-modifying drugs or prophylactic drugs for Parkinson's disease, candidate drugs or therapeutic methods, regenerative cell therapy etc., it is possible to evaluate the effect of suppressing the progression of Parkinson's disease by the therapeutic method. Today, mainstreams to evaluate the effects of disease-modifying drugs in Parkinson's disease are quantitative methods for neurological deficits by invasive and expensive diagnostic imaging methods such as PET and SPECT, which measure dopamine receptors and dopamine transporters in the brain, and motor symptom assessment by clinicians, which is subjective and not necessarily accurate as described above. Alpha-synuclein-related substances, which are considered to be causative agents of Parkinson's disease, are also being studied as biomarkers in blood, but there are not necessarily established indicators. The lack of such biomarkers for evaluating the progression of the disease makes it difficult to evaluate disease-modifying drugs, and is one of the reasons why a radical treatment for Parkinson's disease has not yet been established. Under such circumstances, eye movement indicators directly assess brain dopamine function, and it is possible to measure frequently, continuously, objectively, and easily, therefore, can be used as a means of quantitatively evaluating the progress of Parkinson's disease. Therefore, such an embodiment is expected to be useful for evaluating the action of disease-modifying drugs.

Also, in one example of this embodiment, the user whose eye movements are being measured need not be a patient with a confirmed Parkinson's disease diagnosis. For example, suspected cases of Parkinson's disease and people who are considered to be at high risk of developing Parkinson's disease can be targeted. Suspected cases of Parkinson's disease include, for example, patients who actually have parkinsonism syndromes such as multiple system atrophy, corticobasal degeneration, supranuclear progressive palsy, and the like, and patients with essential tremor, and the like, in addition to Parkinson's disease. Diseases other than Parkinson's disease are known to have limited responsiveness to L-DOPA. In such users, by measuring eye movement, the in vivo dopamine amount and motor symptoms can be estimated, and furthermore, by measuring responsiveness to Parkinson's disease therapeutic drugs such as L-DOPA, it can be applied as a biomarker that assists disease differentiation and prompts appropriate treatment. In addition, people who are considered to be at high risk of developing Parkinson's disease include, for example, those with mutations in hereditary Parkinson's disease-causing genes (such as SNCA, PARK2, PINK1, etc.), those with olfactory disorders, those with REM sleep behavior disorder, and relatively elderly people aged 60 to 70 or older, and the like. In such users, the dopamine amount in vivo is estimated by measuring eye movement, and further, the decline of dopamine function before the onset of Parkinson's disease is detected by measuring responsiveness to Parkinson's disease therapeutic agents such as L-DOPA, this can be applied as a biomarker to enable early diagnosis. It is said that motor symptoms appear in patients with Parkinson's disease after about 60% of the dopaminergic neurons have already been lost, and even if there were disease-modifying drugs that were effective in neuroprotection, their effectiveness would be limited after neurological deficits to the extent that motor symptoms occur. In other words, in order to realize a radical therapy for Parkinson's disease, the difficult problem of discovering and treating patients before the onset of Parkinson's disease is facing. Although it is not yet clear to what extent the dopaminergic neurons that cause abnormal eye movements are lost, since it is thought to reflect dopamine function more directly, there is a high possibility that abnormalities can be identified earlier than general motor symptoms, in addition, it is supposed that the possibility of discrimination is further increased by performing a thorough analysis as shown in this disclosure. In other words, there is a possibility that users with abnormal eye movements themselves are in a high-risk group for Parkinson's disease.

Therefore, in such an example, it becomes possible to screen patients from an early stage in which Parkinson's disease does not appear as motor symptoms, and to prescribe disease-modifying drugs at a stage when efficacy can be expected. In addition, as broader advantages of such an embodiment, for example, if a therapeutic method that can be a preventive measure against Parkinson's disease, such as vaccine therapy against alpha-synuclein, antibody medicine, or nucleic acid medicine, is developed, administering it to all elderly people is not realistic from a medical economic point of view, however, if high-risk or very early-stage Parkinson's disease patients can be screened by eye movement, health-economically optimal prescribing can be achieved.

### NOTE

As described above, the present disclosure is exemplified by the use of its preferred embodiments. However, it is understood that the scope of the present disclosure should be interpreted solely based on the Claims. It is also understood that any patent, any patent application, and any references cited herein should be incorporated herein by reference in the same manner as the contents are specifically described herein. The present application claims priority to Japanese Patent Application No. 2021-40515 filed on Mar. 12, 2021 with the Japan Patent Office, the contents of which are incorporated by reference as though they constitute the subject matter of the present application in its entirety.

### INDUSTRIAL APPLICABILITY

The present disclosure provides techniques for estimating or predicting the condition of Parkinson's disease patients and providing treatment to improve quality of life.

### DESCRIPTION OF SYMBOLS

10, 10′, 1000 system
11 information obtaining means
12 calculation means
13 estimation means
100 user device
200 server device
300 database section
400 network

1. A method for estimating or predicting the presence or absence, amount or level of in vivo dopamine or a substance biologically equivalent to dopamine in a subject, or a variation thereof, based on ocular information in the subject.

2. The method according to claim 1, wherein the in vivo dopamine or the substance biologically equivalent to dopamine includes dopamine or a substance biologically equivalent to dopamine in the brain.

3. The method according to claim 1 or 2, wherein the ocular information includes at least parameters relating to blinking.

4. A method of evaluating a therapeutic or prophylactic agent or other medical technology for a patient with Parkinson's disease who is being treated with L-DOPA or an L-DOPA-related compound or a dopamine agonist, comprising:
A) a step of obtaining ocular information of the patient; and
B) a step of calculating an estimated effective amount or effective level of the therapeutic or prophylactic agent or other medical technology from the ocular information.

5. The method according to claim 4, wherein the step B) comprises:
inputting the ocular information into a trained model, wherein the trained model has trained a correlation between the ocular information and the estimated effective amount or effective level of the therapeutic or prophylactic agent or other medical technology; and
obtaining an output from the trained model.

6. The method according to claim 4, wherein the step B) comprises:
a) a step of calculating the estimated presence or absence, amount or level of in vivo dopamine or a substance biologically equivalent to dopamine of the patient based on the ocular information; and
b) a step of calculating an estimated effective amount or effective level of the therapeutic or prophylactic agent or other medical technology from the presence or absence, amount or level, or a variation thereof.

7. The method according to claim 6, wherein the step a) comprises:
inputting the ocular information into a trained model, wherein the trained model has trained a correlation between the ocular information and the presence or absence, amount or level of the in vivo dopamine or the substance biologically equivalent to dopamine; and
obtaining an output from the trained model.

8. The method according to according to any one of claims 4 to 7, wherein the therapeutic or prophylactic agent or other medical technology includes L-DOPA or an L-DOPA-related compound, a dopamine agonist, a L-DOPA adjunct, a dopamine neuronal function restoring agent, a dopamine producing cell medicine, a dopamine producing gene therapy, or a surgical therapy.

9. The method according to claim 8, wherein the surgical therapy includes deep brain stimulation or stereotactic ablation.

10. The method according to according to any one of claims 4 to 9, further comprising
C) a step of estimating the effect of a medicine having an improving effect on L-DOPA or an L-DOPA-related compound or a dopamine agonist in Parkinson's disease patients being treated with L-DOPA or an L-DOPA-related compound or a dopamine agonist based on the estimated effective amount or effective level.

11. The method according to claim 10, wherein the effect of the medicine includes suppressing flactuation and temporal decreases in dopamine amounts in synaptic clefts in the striatum or dopamine agonists when L-DOPA, L-DOPA-related compounds, or dopamine agonists are administered.

12. A method of estimating or predicting the condition of a Parkinson's disease patient being treated with L-DOPA or an L-DOPA-related compound or a dopamine agonist, comprising
A) a step of obtaining ocular information of the patient; and
B) a step of estimating or predicting the patient's condition based on the ocular information.

13. The method according to claim 12, wherein the step B) comprises:
inputting the ocular information into a trained model, wherein the trained model has trained a correlation between the ocular information and the patient condition; and
obtaining an output from the trained model.

14. The method according to claim 12, wherein the step B) comprises:
a) a step of calculating the estimated presence or absence, amount or level of in vivo dopamine or a substance biologically equivalent to dopamine of the patient based on the ocular information; and
b) a step of estimating or predicting the patient's condition from the presence or absence, amount or level, or a variation thereof.

15. The method according to claim 14, wherein the step a) comprises:
inputting the ocular information into a trained model, wherein the trained model has trained a correlation between the ocular information and the presence or absence, amount or level of the in vivo dopamine or a substance biologically equivalent to dopamine; and
obtaining an output from the trained model.

16. The method according to according to any one of claims 12 to 15, wherein the condition includes the presence or absence or degree or score of at least one selected from the group consisting of dyskinesia, wearing off, ON-OFF, no-on phenomenon, delayed-on phenomenon, akinesia, resting tremor, muscle rigidity, postural instability, forward leaning posture, freezing phenomenon, sleep disorders,
mental/cognitive/behavioral disorders, autonomic disorders and sensory disorders.

17. The method according to claim 16, wherein the step B) comprises:
estimating or predicting the presence or absence or degree or score of symptoms of Parkinson's disease or the presence or absence or degree or score of dyskinesia in the patient based on the ocular information.

18. The method according to claim 15, wherein step b) comprises:
estimating or predicting the patient's condition by comparing an output from the model to one or more thresholds set for the patient.

19. The method according to claim 18, further comprising a step of calculating one or more thresholds set for the patient.

20. The method according to claim 19, wherein the step of calculating one or more thresholds set for the patient comprises:
obtaining ocular information in the baseline condition of the patient prior to the step A); and
calculating the threshold from the ocular information in the baseline condition.

21. The method according to any one of claims 4 to 20, wherein the step A) comprises:
obtaining at least one ocular information source; and
extracting the ocular information from the ocular information source.

22. The method according to claim 21, wherein the at least one ocular information source includes optical, physical or electrical information on the muscle or surrounding skin involved in eye or eye movement of the patient, or a combination thereof.

23. The method according to claim 22, wherein obtaining the at least one ocular information source includes using at least one selected from the group consisting of an image analysis method, a method using reflected light, a distance measurement method, an electrooculography method, a search coil method, and a probe method, to obtain the at least one ocular information source.

24. The method according to claim 22 or 23, wherein extracting the ocular information from the ocular information source includes deriving from the ocular information source a first parameter relating to the eye of the patient and deriving a second parameter relating to the eye of the patient by treating the first parameter, and wherein the ocular information includes the first parameter and/or the second parameter.

25. The method according to claim 24, wherein the first parameter includes at least one selected from the group consisting of blink frequency or number of blinks, blink duration, time between blinks, eye closing time, eye opening speed, eye closing speed, eyelid movement width, eyelid opening, and movement distance, movement direction, speed, acceleration, angular velocity, saccade, gliding eye movement, vestibulo-ocular reflex, convergence/divergence, and fixational tremor (ocular tremor, drift, micro saccade) for spontaneous blink, voluntary blink, or reflex blink.

26. The method according to claim 24 or claim 25, wherein the second parameter includes at least one selected from the group consisting of a function output with the first parameter as an input variable, an intra-division calculated value that is a calculated value in each division when the data of the first parameter is divided into a plurality of divisions by a predetermined threshold, and an inter-division calculated value that is a calculated value of the intra-division calculated value of the plurality of divisions.

27. The method according to claim 26, wherein the intra-division calculated value includes various statistics such as maximum value, minimum value, mean value, median value, dispersion, 25% percentile value, 75% percentile value, frequency analysis spectrum, and the like.

28. The method according to claim 26 or claim 27, wherein the inter-division calculated value includes weighted sums, differences, time differentiations, time integrals, ratios, correlation coefficients, and covariances.

29. The method according to claim 24, wherein the first parameter includes blink duration, and the second parameter includes blink frequency classified based on blink duration, or long blink frequency and short blink frequency, or the ratio of long blink frequency to short blink frequency.

30. The method according to claim 29, further comprising obtaining criteria for dividing the blink duration from the ocular information source.

31. The method according to claim 30, wherein obtaining criteria for dividing the blink duration from the ocular information source includes deriving time-series data of blink frequency and blink duration of each blink from an ocular information source obtained from the patient or another patient, deriving the time-series data of blink frequency into a plurality of data according to the length of the blink duration, calculating a degree of similarity between each of the plurality of classified data and other data among the plurality of classified data adjacent to each other in the blink duration, and setting a threshold for dividing the blink duration based on the similarity.

32. A system for evaluating therapeutic or prophylactic agent or other medical technology for Parkinson's disease patients being treated with L-DOPA or an L-DOPA-related compound or a dopamine agonist, comprising
a means for acquiring ocular information of the patient, and
a means for calculating an estimated effective amount or effective level of the therapeutic or prophylactic agent or other medical technology from the ocular information.

33. A system for estimating or predicting the condition of a Parkinson's disease patient being treated with L-DOPA or an L-DOPA-related compound or a dopamine agonist, comprising
a means for obtaining ocular information of the patient; and
a means for estimating or predicting the patient's condition based on the ocular information.

34. The system according to claim 32 or 33, wherein the system is a user device.

35. The system according to claim 34, wherein the user device is one information processing device selected from the group consisting of smart phones, tablet computers, smart glasses, smart watches, laptop computers, and desktop computers.

36. The system according to claim 34 or claim 35, wherein the user device comprises a portion that implements the function of measuring optical information and/or electro-oculography of the muscle or peripheral skin involved in eye or eye movement.

37. The system according to claim 32 or claim 33, wherein the system includes a user device and a server device, the user device includes an information obtaining means, a transmission means for transmitting the ocular information to the server device, and a receiving means for receiving the result estimated by the estimation means from the server device, and the server device includes a receiving means for receiving the ocular information from the user device, the calculation means, the estimating means, and a transmitting means for transmitting the result estimated by the estimating means to the user device.

38. The system according to claim 32 or claim 33, wherein the system includes a user device and a server device, the user device includes the information obtaining means, the calculation means, a transmission means for transmitting the calculated presence/absence, amount or level to the server device, and a receiving means for receiving the result estimated by the estimation means from the server device, and the server device includes a receiving means for receiving the calculated presence/absence, quantity or level from the user device, the estimating means, and a transmitting means for transmitting the result estimated by the estimating means to the user device.

39. A program for evaluating therapeutic or prophylactic agent or other medical technology for Parkinson's disease patients being treated with L-DOPA or an L-DOPA-related compound or a dopamine agonist, wherein the program is run on a computer system having a processor, and the program causes the processor to perform processing including
A) a step of obtaining ocular information of the patient; and
B) a step of calculating from the ocular information an estimated effective amount or effective level of the therapeutic or prophylactic agent or other medical technology.

40. A program for estimating or predicting the condition of Parkinson's disease patients being treated with L-DOPA or an L-DOPA-related compound or a dopamine agonist, wherein the program is run on a computer system having a processor, and the program causes the processor to perform processing including
A) a step of obtaining ocular information of the patient; and
B) a step of estimating or predicting the condition of the patient based on the ocular information.

41. The program according to claim 39 or claim 40, wherein the computer system is a user device.

42. The program according to claim 39 or claim 40, wherein the computer system is a server device.

43. A method for managing the health of a Parkinson's disease patient, comprising: performing the method according to claim 1, 4 or 12; and applying a treatment to the Parkinson's disease patient if the method determines that the Parkinson's disease patient should be given an additional treatment.

44. A method for managing the health of a patient with Parkinson's disease, comprising:
performing the method according to claim 1, 4 or 12; and
applying L-DOPA or an L-DOPA-related compound or a dopamine agonist, an L-DOPA adjunct, a dopamine neuronal function restoring agent, a dopamine producing cell medicine, or a dopamine producing gene therapy, or a surgical therapy to the Parkinson's disease patient when it is determined that the L-DOPA or an L-DOPA-related compound or a dopamine agonist, an L-DOPA adjunct, a dopamine neuronal function restoring agent, a dopamine producing cell medicine, or a dopamine producing gene therapy, or a surgical therapy should be applied to the Parkinson's disease patient, by the method.

45. The method according to claim 44, wherein the surgical therapy comprises deep brain stimulation or stereotactic ablation.

46. A method for managing the health of a Parkinson's disease patient, comprising: performing the method according to claim 1, 4 or 12; and issuing an alert regarding a treatment if the method determines that the Parkinson's disease patient should be given an additional treatment.

47. A system for health management of a patient with Parkinson's disease, comprising
an estimation or prediction system configured to be able to execute the method according to claim 1, 4 or 12, and
a means for applying a treatment to the Parkinson's disease patient if the estimation or prediction system determines that the Parkinson's disease patient should be given an additional treatment.

48. A system for health management of a Parkinson's disease patient, comprising
an estimation or prediction system configured to be able to perform the method according to claim 1, 4 or 12; and
a means for applying L-DOPA or an L-DOPA-related compound or a dopamine agonist, an L-DOPA adjunct, a dopamine neuronal function restoring agent, a dopamine producing cell medicine, or a dopamine producing gene therapy, or a surgical therapy to the Parkinson's disease patient when it is determined that the L-DOPA or an L-DOPA-related compound or a dopamine agonist, an L-DOPA adjunct, a dopamine neuronal function restoring agent, a dopamine producing cell medicine, or a dopamine producing gene therapy, or a surgical therapy should be applied to the Parkinson's disease patient, by the estimation or prediction system.

49. The system according to claim 48, wherein the surgical therapy comprises deep brain stimulation or stereotactic ablation.

50. A system for health management of a patient with Parkinson's disease, comprising
an estimation or prediction system configured to be able to perform the method according to claim 1, 4 or 12, and
a means for issuing an alert relating to a treatment if the estimation or prediction system determines that an additional treatment should be applied to the Parkinson's disease patient.

51. A program for the health care of patients with Parkinson's disease, wherein the program is run on a computer system having a processor, and the program causes the processor to perform a processing including
performing the method according to claim 1, 4 or 12; and
issuing an instruction to perform a treatment on the Parkinson's disease patient if the method determines that the Parkinson's disease patient should be given an additional treatment.

52. A program for health care of a patient with Parkinson's disease, wherein the program is run on a computer system comprising a processor, and the program causes the processor to perform a processing including
performing the method according to claim 1, 4 or 12; and
issuing an instruction to perform L-DOPA or an L-DOPA-related compound or a dopamine agonist, L-DOPA adjuncts, dopamine neuronal function restoring agents, dopamine producing cell therapy or dopamine producing gene therapy, or if it is determined that a surgical therapy should be applied, to perform L-DOPA or an L-DOPA-related compound or a dopamine agonist, L-DOPA adjuncts, dopamine neuronal function restoring agents, dopamine producing cell therapy or dopamine producing gene therapy, or surgical therapy on the Parkinson's disease patient.

53. The program according to claim 52, wherein the surgical therapy comprises deep brain stimulation or stereotactic ablation.

54. A program for the health care of patients with Parkinson's disease, wherein the program is run on a computer system having a processor, and the program causes the processor to perform a processing including
performing the method according to claim 1, 4 or 12;
and issuing an alert relating to a treatment if the method determines that an additional treatment should be applied to the Parkinson's disease patient.
